(19) United States
(12) Patent Application Publication
(10) Pub. No.: US 2026/0345487 A1
(43) Pub. Date: Nov. 5, 2026

(54) COMPOSITION COMPRISING AQUEOUS PLANT EXTRACT AND/OR ESSENTIAL OIL AND CAFFEINE AND APPLICATION THEREOF FOR IMPROVING COGNITIVE AND MOOD FUNCTIONS

(71) Applicant: THE COCA-COLA COMPANY, Atlanta, GA (US)

(72) Inventors: Bin WANG, Atlanta, GA (US); Haiyu REN, Atlanta, GA (US); Scott MICHALUK, Atlanta, GA (US); Yu SHI, Atlanta, GA (US)

(21) Appl. No.: 19/119,909

(22) PCT Filed: Oct. 16, 2023
(86) PCT No.: PCT/US2023/077000

§ 371 Date: Apr. 10, 2025

Related U.S. Application Data
(60) Provisional application No. 63/379,565, filed on Oct. 14, 2022.

(51) Int. Cl. A61K 9/00; A61K 31/05; A61K 31/522; A61K 36/537; A61K 36/61; A61K 36/82; A61K 36/87; A61P 25/26

(52) U.S. Cl. CPC A61K 9/0095; A61K 31/05; A61K 31/522; A61K 36/537; A61K 36/61; A61K 36/82; A61K 36/87; A61P 25/26

(57) ABSTRACT

The present disclosure presents compositions and methods for improving the condition of a subject, including improving cognitive function and mood effects. The composition includes a first polyphenol and at least one essential oil(s), wherein a ratio of total polyphenol to total essential oil is from about 1:10 to about 100:1. The method includes administering to the human in need of an improved condition an edible composition, wherein the composition comprises: a first polyphenol and at least one essential oil(s), wherein a ratio of total polyphenol to total essential oil is from about 1:10 to about 100:1.

### CROSS REFERENCE TO RELATED APPLICATIONS

This application is being filed as a PCT International patent application and claims the benefit of and priority to U.S. Provisional Patent Application Ser. No. 63/379,565 filed Oct. 14, 2022, the subject matter of which is hereby incorporated by reference in its entirety.

### INTRODUCTION

The present disclosure generally relates to edible compositions, beverages, and methods for improving a condition or function or performance of a human.

Cognitive functions and mental energy are generally important for improving performance of human activity. Consumption of caffeine or caffeine-containing drinks is often effective as a short-term strategy to improve cognitive performance and mental energy. However, concerns have been raised about the potential disadvantages of too much caffeine consumption, and common side effects of large doses of caffeine among susceptible individuals include anxiety, cardiac symptoms, and insomnia. Increased attention is paid to edible products with beneficial ingredients other than caffeine, either alone or in combination with smaller amounts of caffeine that are typically consumed.

Psychological effects of polyphenols derived from plant extracts have been studied. Jackson et al. (Nutritional Neuroscience, 2021) reported that consumption of coffee berry extract and/or apple extract could lead to improvement of mood and increase of cerebral blood flow in healthy humans. The positive effect is attributed to polyphenols from coffee berry (chlorogenic acid) and apple (flavanol). Polyphenols have been known to have both anti-inflammatory and antioxidant properties, leading to positive mood changes.

Ward-Ritacco et al. (Journal of Cognitive Enhancement, 2021) reported that moderate doses of caffeine combined with polyphenols derived from apple extract are effective at improving cognitive outcomes, especially serial seven subtraction, and mood responses to the tasks, including alertness and mental and physical fatigue. However, Ward-Ritacco et al. found that very low doses of caffeine (10 to 20 mg) combined with apple polyphenols are not consistently associated with improvements in cognitive, motivation, or mood outcomes.

In contrast, a series of recent publications cast doubts on the effectiveness of polyphenols. For example, Reed et al. (Journal of Cognitive Enhancement, 2019) reported a study on the influence of acute consumption of a polyphenol-rich, non-caffeinated coffeeberry extract on performance of a series of fatiguing cognitive tasks. Reed et al. concluded from the results that the coffeeberry extract beverages had no effect on self-reported motivation to complete the cognitive tasks or either working memory or sustained attention performance.

More recently, McGranahan et al. (Current Developments in Nutrition, 2021) reported that consumption of a beverage with 100 mg or 300 mg coffeeberry extract one hour before a cognitive test or two hours before a high intensity exercise bout does not influence feelings of alertness, energy, and fatigue or cycling performance.

In spite of the above disclosures, it is still desired for new compositions and beverages that further improve cognitive functions, mental energy and mood compared to conventional caffeinated products or decaffeinated products. It is also desired for methods of improving cognitive functions, mental energy and mood with compositions or beverages for certain subject pools in need thereof. It is further desired for methods for improving cognitive functions, mental energy and mood using compositions or beverages that have identified ingredients and optimized contents thereof.

### Methods and Compositions for Improving Cognitive and Mood Functions

The present disclosure presents compositions, beverages, and methods that meet the above stated needs.

In some aspects, the present disclosure relates to an edible composition. In one example, an edible composition comprises: a first polyphenol; and at least one essential oil(s), wherein a ratio of the total polyphenol to the total essential oil(s) is from about 1:10 to about 100:1.

In some embodiments the at least one essential oils(s) are selected from terpenes, monoterpenes, sesquiterpenes, diterpenes, esters, aldehydes, ketones, alcohols, phenols and oxides, and derivatives and/or any combinations thereof.

In some embodiments the first polyphenol and the least one essential oil(s) are derived from the same plant species, while in other embodiments the first polyphenol and the at least one essential oils(s) are derived from different plants species.

In additional embodiments the at least one essential oil(s) comprise a first essential oil and a second essential oil. In this embodiment the first essential oil is derived from the same or different plant species as the first polyphenol, and the second essential oil is derived from a different plant species than the first essential oil.

In some embodiments the at least one essential oil(s) are derived from a plant genus selected from Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselinum, Pimpinella, Piper, Thymus, Zingiber, Humulus, or any combinations thereof.

In embodiments, the first polyphenol is derived from a plant genus selected from Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Salvia, Satureia, Schimis, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja or any combination thereof.

In some embodiments the first polyphenol is selected from a group comprising a flavonoid, a phenolic acid, a curcuminoid, a lignan, a stilbene, a phenolic terpene, a glycoside, a glucuronide, a depside or any combination thereof.

A flavonoid is selected from a group comprising a flavanol, a flavone, a flavonol, a flavanone, an isoflavone, an anthocyanidin, a gallate, a glycoside, a polymer thereof or any combination thereof.

A flavanol is selected from a group comprising a catechin, an epicatechin, a catechin gallate, an epicatechin gallate, a gallocatechin, an epigallocatechin, a gallocatechin gallate, an epigallocatechin gallate, a proanthocyanidin or any combinations thereof.

A flavone is selected from a group comprising a luteolin, an apigenin, a tangeritin, a chrysin, or any combination thereof.

A flavanol is selected from a group comprising a quercetin, a kaempferol, a myricetin, a rutin, and any combination thereof.

A flavanone is selected from a group comprising a eriodictyol, a hesperetin, a narigenin, or any combination thereof.

An isoflavone is selected from a group comprising a genistein, a daidzein orany combination thereof.

An anthocyanidin is selected from a group comprising a cyanidin, a delphinidin, a malvidin, a pelargonidin, an aurantinidin, a capensinidin, an europinidin, a hirsutidin, a peonidin, a petunidin, a pulchellidin, a rosinidin and or combination thereof.

A phenolic acid is selected from a group comprising a rosmarinic acid, a vanillic acid, a caffeic acid, a gallic acid, a protocatechuic acid, a salicyclic acid, a ferulic acid, a sinapic acid, a chlorogenic acids, a coumaric acid or any combination thereof.

A curcuminoid is selected from a group comprising a bisdemethoxycurcumin, a demethoxycurcumin, a curcumin, or any combination thereof.

A lignan is selected from a group comprising a lariciresinol, a pinoresino, a matairesinol, a syrigaresinol, a sesamin, a sesaminol, and any combination thereof. A stilbene is selected from a group comprising a resveratrol, a pterostilbene, and any combination thereof.

A phenolic terpene is selected from a group comprising a carnosic acid, a rosmanol, a carnosol, or any combination thereof.

In some embodiments, the composition comprises sage (Salvia officinalis and/or Salvia lavandulifolia). In this embodiment the first polyphenol and at the at least one essential oil(s) compound are both derived from sage. In further embodiments the first polyphenol is derived from Salvia officinalis and the at least one essential oil(s) is derived from Salvia lavandulifolia. In some embodiments, the sage is selected from a sage extract, an aqueous sage extract, a sage concentrate, sage oil, or a sage juice.

In some embodiments, the composition comprises grape seed. In this embodiment the first polyphenol is derived from grape seed. In some embodiments, the grape seed is selected from a grape seed extract, an aqueous grape seed extract, a grape seed concentrate, grape seed oil, or a grape seed juice.

Further embodiments additionally incorporate a second polyphenol. The second polyphenol is derived from a different plant genus or species than the first polyphenol. In some embodiments, the second polyphenol comprises comprising a flavonoid, a phenolic acid, a curcuminoid, a lignan, a stilbene, a phenolic terpene, a glycoside, a glucuronide, a depside or any combination thereof.

In some embodiments, the second polyphenol is not derived of sage or grape seed. In some embodiments, the second polyphenol is derived from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof.

In some embodiments the second polyphenol is derived from a plant genus selected from Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselimam, Pimpinella, Piper, Thymus, Zingiber, Aloe, Bacopa, Daucus, Euterpe, Humulus, Juniperus, Malpighia, Morus, Phyllanthus, Rubus, Satureja.

In some embodiments, the composition further comprises a sweetener. In some embodiments, the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs), and combinations thereof.

In some embodiments, the composition further comprises at least one additive or functional ingredient or both.

In some embodiments, the composition comprises an essential oil, caffeine, a first polyphenol, and optionally a second polyphenol. The first and second polyphenols may be selected from the group consisting of Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.

In embodiments, he essential oil may be selected from the group consisting of Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselinum, Pimpinella, Piper, Thymus, Zingiber, Humulus or any combinations thereof.

In embodiments, the Camellia species may be in an amount of about 100 mg to about 4.0 g, about 300 mg to about 2.0 g, about 500 mg to about 1.0 g, or about 700 mg to about 900 mg.

In embodiments, the Salvia species may be in an amount of about 100 mg to about 500 mg, about 50 mg to about 700 mg, about 200 mg to about 300 mg, or about 400 mg to about 500 mg.

In embodiments, the caffeine may be in an amount greater than zero, but less than 10 mg, less than 20 mg, less than 30 mg, less than 40 mg, less than 50 mg, less than 60 mg, less than 75 mg, less than 100 mg, or less than 150 mg.

In some embodiments, the composition is in a drinkable liquid form, a concentrate form, a dry form, or a semi-dry form. In some embodiments, the composition is a gum, gel, tablet, capsule, granule, cubic, or dry powder.

In some embodiments, the composition is a beverage selected from the group of non-carbonated beverage, carbonated beverage, juice beverage, fruit juice, coffee beverage, tea beverage, milk beverage, diary beverage, plant protein drink, plant-based beverage, sport drink, energy drink. In some embodiments, the composition has a Brix value of about 1° to about 25°, or about 5° to about 20°, or about 7° to about 15°.

In some embodiments, the composition has a total content of polyphenol from about 0.002 wt % to about 2 wt %, or from about 0.05 wt % to about 1 wt %, or from about 0.1 wt % to about 0.5%.

In some embodiments, the composition has a dosage of total polyphenol from 15 mg to about 300 mg, or from about 30 mg to about 200 mg, or from about 45 mg to about 150 mg for one serving.

In some embodiments, the weight ratio of the total polyphenol to the total essential oil(s) is from about 1:10 to about 100:1, or from about 1:9 to about 90:1, or from about 1:8 to about 80:1, from about 1:7 to about 70:1, from about 1:6 to about 60:1, from about 1:5 to about 50:1, or from about 1:4 to about 40:1, or from about 1:3 to about 30:1, or from about 1:2 to about 20:1.

In some embodiments, the weight ratio of total caffeine to the total plant extract composition is from 1:300 to 1:1, or from about 1:275 to about 1:2, or from about 1:250 to about 1:3, or from about 1:225 to about 1:4, or from about 1:200 to about 1:5, or about 1:200 to about 1:20, or from about 1:175 to about 1:6, or from about 1:150 to about 1:7, or from about 1:125 to about 1:8, or from about 1:100 to about 1:9, or from about 1:50 to about 1:10, or about or any range in between.

In some embodiments, the ratio of the at least one essential oil(s) to the at least one aqueous extractive(s) is from about 1:10 to about 1:800, from about 1:8 to about 1:600, from about 1:6 to about 1:400, from about 1:4 to about 1:200, or from about 1:2 to about 1:100, or from about 1:100 to about 1:800 or about 1:200 to about 1:800, or about 1:10 to about 1:4, or any ranges in between.

In some embodiments, the composition has a total essential oil(s) from about 0.001 wt % to about 1 wt %, or from about 0.05 wt % to about 1 wt %, or from about 0.1 wt % to about 0.5%.

In some embodiments, the composition has a dosage of at least one essential oil(s) from 3 mg to about 150 mg, or from about 10 mg to about 100 mg, or from about 25 mg to about 50 mg for one serving.

In some embodiments, the first polyphenol and the at least one essential oil(s) are derived from sage extract and the dosage of sage extract is from about 50 mg to about 1,000 mg, or from about 100 mg to about 800 mg, or from about 200 mg to about 600 mg for one serving. More specifically, in these embodiments, the sage extract is the source of and contains both the first polyphenol and at least one of the essential oil(s) within the composition.

In some embodiments, the first polyphenol is derived from sage and the composition has a weight ratio of the first polyphenol derived from sage to the total essential oil(s) from about 2:1 to about 200:1. In some embodiments, the first polyphenol comprises at least one of: luteolin glycoside, luteolin glucuronide, and rosmarinic acid.

In some embodiments, the first polyphenol is derived from grape seed extract and the composition has a dosage of grape seed extract from about 50 mg to about 1,000 mg, or from about 100 mg to about 800 mg, or from about 200 mg to about 600 mg for one serving. More specifically, in these embodiments, the grape seed extract is the source of the first polyphenol but contains other compounds as well. In these embodiments where the first polyphenol is derived from grape seed, the composition has a weight ratio the first polyphenol to the total essential oil(s) from about 2:1 to about 200:1. In some embodiments, the first polyphenol comprises at least one of: catechins, and procyanidins.

In other embodiments, a beverage is disclosed. The beverage comprises a first polyphenol and the at least one essential oil(s), wherein the beverage has a weight ratio of the total polyphenol to the total essential oil(s) from about 1:10 to about 100:1. The first polyphenol in this embodiment is derived from sage. In some embodiments, the beverage further comprises a second polyphenol not derived from of sage. In some embodiments, the second polyphenol is from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof. In some embodiments, the product of sage is a sage extract.

In yet another embodiment, a beverage is disclosed comprising a first polyphenol and at least one essential oil(s), wherein the beverage has a weight ratio of the total polyphenol to the total essential oil(s) from about 1:1 to about 100:1. In this embodiment the first polyphenol is derived from grape seed.

In some embodiments, the beverage further comprises a second polyphenol not derived from the product of grape seed. In some embodiments, the second polyphenol is from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof. In some embodiments, the product of grape is a grape seed extract.

In another aspect, the present disclosure provides a method for improving a condition of a human. The method involves administering to the human in need of an improved condition an edible composition. The composition comprises a first polyphenol and at least one essential oil(s), wherein a ratio of the total polyphenol to the total essential oil(s) is from about 1:10 to about 100:1.

In some embodiments, the administration of the composition to the human causes one or more effects on the human: improving cognitive function; improving mood; improving sleep functions, improving alertness; improving contentedness; improving calmness; improving tranquility; improving mental capability; improving memory accuracy; improving speed of memory; improving relaxation; improving/sustaining attention; improving accuracy of attention; improving stroop effect, improving speed of attention; reducing stress; reducing tension; reducing mental fatigue; reducing anxiety; reducing inertia; reducing headache; promoting maintenance of normal sleep, reducing sleep onset latency, improving sleep quality, alleviating of the subjective feeling of jet leg, or any combinations thereof.

In some embodiments, the administration of the composition to the human causes one more effects related to improved mood, including improved happiness, improved friendliness, increased sociability, improved carefree feeling, increased relaxation, increased euphoria and/or increased conviviality.

In some embodiments, the composition takes effect after a time period following the administration, wherein the time period is about 5 minutes, or about 15 minutes, or about 30 minutes, or about 60 minutes, or about 90 minutes, or about 120 minutes or about 150 minutes, or about 200 minutes, or about 250 minutes, or about 300 minutes.

In some embodiments, the composition administered to the human comprises 15 mg, or at least 50 mg, or at least 75 mg, at least 100 mg, or at least 150 mg, or at least 200 mg, or at least 250 mg or at least 300 mg of total polyphenol. In some embodiments, the composition administered to the human comprises at least 3.0 mg, at least 50 mg, at least 100 mg, or at least 150 mg of total essential oil(s).

### DEFINITION AND INTERPRETATION OF SELECTED TERMS

As used herein, “weight percent,” “wt %,” “percent by weight,” “% by weight,” and variations thereof refer to the concentration of a substance as the weight of that substance divided by the total weight of the composition and multiplied by 100. It is understood that, as used here, “percent,” “%,” and the like are intended to be synonymous with “weight percent,” “wt %,” etc.

As used herein, “g” represents gram; “L” represents liter; “mg” represents “milligram (10⁻³ gram);” “mL” or “cc” represents milliliter (10⁻³ liter). One “μL” equals to one micron liter (10⁻⁶ liter). The units “g/100 g,” “g/100 mL,” or “g/L” are units of concentration or content of a component in a composition. One “mg/L” equals to one ppm (part per million). “Da” refers to Dalton, which is the unit for molecular weight; One Da equals to one g/mol. The unit of temperature used herein is degree Celsius (°C).

The term “about” is used in conjunction with numeric values to include normal variations in measurements as expected by persons skilled in the art and is understood to have the same meaning as “approximately” and to cover a typical margin of error, such as ±15%, ±10%, ±5%, ±1%, ±0.5%, or even ±0.1% of the stated value. The term “about” also encompasses amounts that differ due to different equilibrium conditions for a composition resulting from a particular initial composition. Whether or not modified by the term “about,” the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise. As used herein, “and/or” refers to and encompasses any and all possible combinations of one or more of the associated listed items, as well as the lack of combinations when interpreted in the alternative (“or”).

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “substantially free” may refer to any component that the composition of the disclosure lacks or mostly lacks. When referring to “substantially free” it is intended that the component is not intentionally added to compositions of the disclosure. Use of the term “substantially free” of a component allows for trace amounts of that component to be included in compositions of the disclosure because they are present in another component. However, it is recognized that only trace or de minimus amounts of a component will be allowed when the composition is said to be “substantially free” of that component. Moreover, if a composition is said to be “substantially free” of a component, if the component is present in trace or de minimus amounts it is understood that it will not affect the effectiveness of the composition. It is understood that if an ingredient is not expressly included herein or its possible inclusion is not stated herein, the disclosure composition may be substantially free of that ingredient. Likewise, the express inclusion of an ingredient allows for its express exclusion thereby allowing a composition to be substantially free of that expressly stated ingredient.

The term “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 1%, 5%, 10%, 15%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 1%, 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5% or even 1%) detectable activity or amount.

The term “beverage” as used herein means any drinkable liquid or semi-liquid, including for example water, flavored water, soft drinks, fruit drinks, tea-based drinks, juice-based drinks, gel drinks, carbonated or non-carbonated drinks, and alcoholic or non-alcoholic drinks. In some embodiments, a beverage powder may first be mixed with any drinkable liquid or semi-liquid to obtain a beverage.

As used herein, the term “cognitive function” or “cognition” generally refers an aspect of an individual's psychosocial presentation. Cognitive function relates to multiple mental abilities, including learning, thinking, reasoning, remembering, problem solving, decision making, focusing, concentrating, and attention.

As used herein, the term “attention” refers to the ability to direct and focus cognitive activity on specific stimuli. It is the ability or power to concentrate mentally. Sustained attention refers to the state in which attention must be maintained over a period of time.

As used herein, the term “alertness” refers to the mental state of paying close and continuous attention, being watchful and prompt, or being quick to perceive and/or act. It is being vigilantly attentive, mentally responsive and perceptive, and quick.

As used herein, the term “mental fatigue” refers to a state of awareness describing a range of afflictions, associated with mental weakness. Such mental fatigue can manifest itself either as somnolence (decreased wakefulness) or as a general decrease of attention (not necessarily sleepiness). It may also be described as a decreased level of consciousness. Mental fatigue can be caused by continual mental effort and attention on a particular task, as well as high levels of stress or emotion. Basically, any mental process that goes into overload can result in mental fatigue.

As used herein, the term “mood” refers to an individual's temporary state of mind or quality of feeling (an emotional state) at a particular time. Moods differ from simple emotions in that they are less specific, less intense, and less likely to be triggered by a particular stimulus or event. Moods generally have either a positive or negative valence. Mood is an internal, subjective state. Various measurable aspects of mood functions or various mood related parameters include happiness, friendliness, sociability, carefree feeling, relaxation, calmness, euphoria and/or conviviality.

As used herein, the term “relaxation” refers to the act of mentally relaxing or the state of being mentally relaxed.

As used herein, the term “headache” refers to a persistent or lasting pain or discomfort in the head region.

As used herein, the term “sports performance” refers to an endurance sport, sustained high-intensity sport or team sports all requiring physical performance.

As used herein, “coffee berry” refers to the fruit of the coffee tree (Coffea species) in which exocarp and outer mesocarp (i.e., the pulp) surround the inner mesocarp (i.e. the mucilage) and endocarp (i.e., the hull), which in turn surround the seeds (i.e., the beans). Thus, the term coffee berry specifically refers to a whole coffee berry, which may or may not include the stem of the berry.

As used herein, the term “juice product” refers to a beverage product made from juices or combinations of juices with water or other components for human and/or animal nutritional, health-maintenance, health-improvement, and/or recreational purpose. Particularly preferred juice products include those consumed by human.

“Juice” means a liquid that is found in nature in plant materials, or a diluted form of such liquid. A juice as used herein is not produced by solubilizing a plant material. “Extract” is a material that is drawn out or forcibly removed from a plant material, including through heat, chemical or high pressure means. While an extract may be in a liquid, it is not naturally found in liquid form in a plant but instead is a combination of chemical components that are not naturally found in the liquid of a plant.

As used herein, the term “plant extractive”/“plant extract” is a substance made by extracting components from a matric (e.g., a raw material of the plants) by using a solvent (e.g., ethanol, oil, or water).

As used herein, the term “aqueous extractive”/“aqueous extract” means an extract produced by using water, or a mixture of water and polar solvents completely dissolved in water (e.g., an aqueous ethanol solution/a mixture of water and ethanol). Forms of aqueous extractives/aqueous extracts may be solid or liquid depending on the water content.

As used herein, the term “tea infusion” means a tea liquid beverage brewed using water. “Concentrated tea infusion” means to concentrate a tea infusion by partially removing the water. A concentrated tea infusion may contain from about 20% to about 70% extractive and about 80% to about 30% water. In some embodiments, a concentrated tea infusion may contain about 30% of the extractive and about 70% water.

The term “epigallacatechin gallate” or “EGCG” as used herein encompasses the catechin concentrated for use in examples 2 and 3, and throughout this application. EGCG has a strong antioxidant capacity and potential to reduce stress and inflammation. EGCG is a polyphenol, and is found particularly in Camellia species.

As used herein, the term “nutrient” refers to a compound or mixture of compounds that is/are ingested and provides an alimentary benefit to the person ingesting the compound or mixture of compounds. Thus, the term nutrient as used herein specifically includes a compound or mixture of compounds that provide energy via metabolism of the compound or mixture of compounds (e.g., polysaccharides), interacts with the nervous system and/or immune system to modulate, and preferably stimulate, the nervous system and/or immune system (e g., caffeine), or provides a protective function (e.g., polyphenols as antioxidant).

The term “polyphenol” as used herein refers to a diverse group of compounds produced by a plant, wherein the compounds include a phenol ring to which at least one OH group, and more typically at least two OH groups are covalently attached. Polyphenol used herein encompasses subclasses: flavonoids, phenolic acids, lignans, curcuminoids, phenolic terpene, and stilbenes.

“Flavonoids” as used herein refers a class of secondary plant metabolites that have a general 15-carbon skeleton structure which comprises two phenyl rings and a heterocyclic ring. “Flavonoids” encompass flavanol, flavone, flavonol, flavanone, isoflavone, anthocyanin, or gallate thereof, or glycoside thereof, or oligomer/polymer thereof. Non-limiting examples of flavanol include catechin, epicatechin, gallocatechin, epigallocatechin, and gallates, theaflavin, theaflavin-3-gallate, theaflavin-3′-gallate, theaflavin-3,3′-gallate, thearubigin, procyanidin, cinnamtanni, or gallate thereof. Non-limiting examples of flavone include apigenin, luteolin, chrysoeriol, isorhoifolin, hispidulin, tangeretin, or glycoside thereof. Non-limiting examples of flavonol include quercetin, isorhamnetin, kaempferol, galangin, myricetin, or glycoside thereof. Non-limiting examples of flavanone include eriocitrin, eriodictyol, hesperetin, hesperidin, narigenin, narirutin, pinocembrin, neoeriocitrin, poncitrin, sakuranetin, or glycoside thereof. Non-limiting examples of isoflavone include genistein, malonylgenistin, daidzein, or glycoside thereof. Non-limiting examples of anthocyanin include cyanidin, delphinidin, malvidin, pelargonidin, or glycoside thereof. Non-limiting examples of anthocyanidin or anthocyanin include cyanidin, delphinidin, malvidin, pelargonidin, petunidin, peonidin, and glycosides thereof. More examples of flavonoids within the context of this disclosure include stilbenoids, dihydroflavonols, anthocyanidins, tannins, flavones, flavan-3-ols, flavan-4-ol, flavan-3,4-diol, homoisoflavonoids, bioflavonoids, isoflavonoids, neoflavonoids, or their derivatives.

“Phenolic acids” as used herein encompass hydroxybenzoic acid or hydroxycinnamic acid. Non-limiting examples of hydroxybenzoic acid include protocatechuic acid, gallic acid, vanillic acid, ellagic acid, salicyclic acid, or any salt or derivative thereof. Non-limiting examples of hydroxycinnamic acid include caffeic acid, ferulic acid, sinapic acid, chlorogenic acid, p-coumaric acid, quinic acid, or any salt or derivative thereof.

“Lignans” as used herein encompass sesamin, sesaminol, sesamol, sesamolin, secoisolariciresinol, pinoresinol, lariciresinol, or any derivative thereof.

“Curcuminoids” as used herein encompass bisdemethoxycurcumin, demethoxycurcumin, curcumin, or any derivative thereof.

“Stilbenes” as used herein encompass resveratrol and pterostinene.

“Phenolic terpenes” as used herein encompass carnosic acid, rosmanol, carnosol, or derivatives thereof.

“Polyphenols” according to the present disclosure can be derived from a plant source including but not limited to green tea, cacao bean, cocoa, whole grain such as sorghum, black chokeberry, common bean, peppermint, oregano, tangelo, parsley, sage, celery seed, common verbena, soy, soybean, caper, saffron, lovage, black elderberry, pepper, black raspberry, blackcurrant, plum, bilberry, blueberry, chestnut, walnut, cranberry, clove, olive, rosemary, spearmint, common thyme, turmeric, sesame seed, grape, grape seed, kale.

Non-limiting examples of plant genus (genera) sources for polyphenols described within the context of this disclosure are as follows:
Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.

Non-limiting examples of plant species sources for polyphenols within the context of this disclosure are as follows:
Camellia sinensis, Camellia assamica, Coffea arabica, Coffea canephora, Coffea eugenioides, Ilex paraguariensis, Ilex latifolia, Ilex vomitoria, Ilex guayusa, Ilex kudingcha, Paullinia cupana, Theobroma cacao, Hypericum perforatum, Beta vulgaris, Glycine max, Ginkgo biloba, Matricaria chamomilla, Moringa oleifera, Magnolia officinalis, Withania somnifera, Aspalathus linearis, Euterpe oleracea, Crocus sativus, Polygonum multiflorum, Polygonum cuspidatum, Ribes spp., Rosa spp., Sophora japonica, Citrus spp., Fragaria spp., Juglans spp., Litchi chinensis, Lycium barbarum, Lycium chinense, Malus spp., Mangifera indica, Murraya spp., Olea europaea, Passiflora incarnata, Prunus spp., Punica spp., Sambucus spp., Syzygium aromaticum, Vaccinium spp., Vitis spp., Aronia melanocarpa, Aronia arbutifolia, Aronia prunifolia, Angelica sinensis, Angelica acutiloba, Angelica archangelica, Hibiscus sabdariffa, Chamomilla recutita, Chamaemelum nobile, Jasminum sambac, Jasminum flexile, Jasminum mesnyi, Jasminum angustifolium, Lavandula spp., Melissa officinalis, Valeriana officinalis, Anthemis nobilis, Amomum subulatum, Cinnamomum burmanni, Cinnamomum cassia, Cinnamomum loureirin, Cinnamomum zeylanicum, Cinnamomum camphora, Coriandrum sativum, Coriandrum tordylium, Elettaria cardamomum, Eucalyptus globulus, Eucalyptus cneorifolia, Eucalyptus dives, Eucalyptus Dumosa, Eucalyptus goniocalyx, Eucalyptus horistes, Eucalyptus kochii, Eucalyptus leucoxylon, Eucalyptus largiflorens, Eucalyptus oleosa, Eucalyptus polybractea, Eucalyptus radiata, Eucalyptus rossii, Eucalyptus sideroxylon, Eucalyptus smithii, Eucalyptus. staigeriana, Eucalyptus tereticornis, Eucalyptus viridis, Capsicum spp., Curcuma longa, Foeniculum vulgare, Salvia officinalis, Salvia lavandulifolia, Salvia rosmarinus (Rosmarinus officinalis), Salvia hispanica, Salvia columbariae, Salvia polystachya, Salvia bowleyana, Salvia cavaleriei, Salvia chinensis, Salvia flava, Salvia prionitis, Salvia sonchifolia, Salvia yunnanensis, Salvia miltiorrhiza, Salvia multicaulis, Salvia divinorum, Satureia hortensis, Satureia montana, Schinus mole, Schinus terebinthifolia, Mentha piperita, Mentha spicata, Mentha citrate, Ocimum basilicum, Ocimum citriodorum, Ocimum kilimandscharicum, Ocimum Americanum, Ocimum gratissimum, Ocimum tenuiflorum, Petroselinum crispum, Petroselinum segetu, Pimpinella anisum, Piper nigrum, Thymus vulgaris, Thymus zygis, Thymus serpyllum, Thymus citriodorus, Thymus praecox, Thymus pseudolanuginosus, Zingiber officinale, Zingiber mioga, Aloe vera, Bacopa monnieri, Daucus carota, Euterpe oleracea, Humulus lupulus, Juniperus spp., Malpighia emarginata, Morus spp., Phyllanthus emblica, Rubus spp., Satureja montana.

Non-limiting examples of plant genus (genera) sources for the essential oil(s) described within the context of this disclosure are as follows:
Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Mentha, Ocimum, Origanum, Petroselinum, Pimpinella, Piper, Thymus, Zingiber, Humulus.

Non-limiting examples of plant species sources for the essential oil(s) described within the context of this disclosure are as follows:
Rosa spp., Sophora japonica, Citrus spp., Fragaria spp., Juniperus spp., Murraya spp., Sambucus spp., Syzygium aromaticum, Angelica sinensis, Angelica acutiloba, Angelica archangelica, Helichrysum arenarium, Helichrysum italicum, Hibiscus sabdariffa, Chamomilla recutita, Chamaemelum nobile, Cymbopogon citratus, Geranium spp., Matricaria chamomilla, Jasminum sambac, Jasminum flexile, Jasminum mesnyi, Jasminum angustifolium, Lavandula spp., Melissa officinalis, Valeriana officinalis, Agave amica, Anthemis nobilis, Amomum subulatum, Cinnamomum burmanni, Cinnamomum cassia, Cinnamomum loureirin, Cinnamomum zeylanicum, Cinnamomum camphora, Coriandrum sativum, Coriandrum tordylium, Elettaria cardamomum, Eucalyptus globulus, Eucalyptus cneorifolia, Eucalyptus dives, Eucalyptus Dumosa, Eucalyptus goniocalyx, Eucalyptus horistes, Eucalyptus kochii, Eucalyptus leucoxylon, Eucalyptus largiflorens, Eucalyptus oleosa, Eucalyptus polybractea, Eucalyptus radiata, Eucalyptus rossii, Eucalyptus sideroxylon, Eucalyptus smithii, Eucalyptus. Staigeriana, Eucalyptus tereticornis, Eucalyptus viridis, Capsicum spp., Curcuma longa, Euonymus phellomanus, Foeniculum vulgare, Litsea cubeba, Salvia officinalis, Salvia lavandulifolia, Salvia rosmarinus (Rosmarinus officinalis), Salvia hispanica, Salvia columbariae, Salvia polystachya, Salvia bowleyana, Salvia cavaleriei, Salvia chinensis, Salvia flava, Salvia prionitis, Salvia sonchifolia, Salvia yunnanensis, Salvia miltiorrhiza, Salvia multicaulis, Salvia divinorum, Satureia hortensis, Satureia montana, Schinus mole, Schinus terebinthifolia, Mentha piperita, Mentha spicata, Mentha citrate, Ocimum basilicum, Ocimum citriodorum, Ocimum kilimandscharicum, Ocimum Americanum, Ocimum gratissimum, Ocimum tenuiflorum, Origanum majorana, Origanum vulgare, Petroselinum crispum, Petroselinum segetu, Pimpinella anisum, Piper nigrum, Thymus vulgaris, Thymus zygis, Thymus serpyllum, Thymus citriodorus, Thymus praecox, Thymus pseudolanuginosus, Zingiber officinale, Zingiber mioga, Humulus lupulus.

Non-limiting examples of plant genus (genera) sources for the naturally occurring caffeine (non-added) described within the context of this disclosure are as follows:
Camellia, Coffea, Theobroma, Ilex, Cola.

Non-limiting examples of plant species sources for the naturally occurring caffeine (non-added) described within the context of this disclosure are as follows:

Camellia species is selected from varieties of Camellia sinensis, for examples C. sinensis var. sinensis and C. sinensis var. assamica. Coffea species is selected from a group comprising Coffea arabica, C. canephora (esp. C. canephora var. robusta), C. eugenioides and combinations thereof. Theobroma species is selected from a group comprising Theobroma cacao and combinations thereof. The Ilex species is selected from a group comprising Ilex paraguariensis, I. latifolia, I. vomitoria, I. guayusa, I. kudingcha and combinations thereof.

“Sage” as used herein refers to a plant in the family of Labiatae/Lamiaceae. “Sage” “common sage” “Sibelius sage” can be used interchangeably according to the present disclosure.

“Extract” of a plant (e.g., sage extract) as used herein refers to a concentrate of physiologically active constituents from the plant of a part thereof extracted using a solvent. An example of how such a concentrate can be produced is given below: Leaves of a plant are mechanically harvested and dried in a hot air drier (continuous process drier; indirect fired heating). Plant extracts may be produced or manufactured using various solvents and technologies including, but not limited to ethanol, butane, methane, carbon dioxide, ice, water, steam. Any compounds or ingredients according to the present disclosure may be extracted from plants bred to express desired profiles for purity. Any compounds or ingredients according to the present disclosure may be purified using supercritical fluid (“SFC”) extraction and similar technologies. In one example, the process of crystallization involves placing the compound of interest in a liquid and then cooling or adding participants to the solution which would lower the solubility of the compound of interest so that it forms crystals. In this example, crystals are then separated from the liquid through filtration or centrifuge.

The term “derived from” as used herein, for example “the first polyphenol is derived from sage”, is used to mean that the component was obtained or extracted from that plant source. The term “derived from” can additionally mean that the component was obtained through synthetic means, for example from a synthetic version, analog or derivative of a naturally extracted component. For example, “an essential oil derived from sage” can mean that the essential oil component was extracted from a sage plant, or the essential oils was synthetically composed and is a derivative or analog of the essential oil derived from said plant. Additionally, within the scope of this application, when the term “derived from” is used for example as “wherein the first polyphenol and the first essential oil(s) are derived from sage extract” this is intended to mean that the sage extract comprises both those components and hence is the source of both the polyphenol and the first essential oil(s) components.

The term “serving” as used herein encompasses “a serving,” “serving size,” or “serving per day,” and “served dose/dosage,” “administration,” “administration dose/dosage,” “administered units.” A “serving” “has its typical meaning as used in the art, and/or represents the amount of the composition that is administered at one moment of intake, for example as one meal or before, during and/or after a meal, before going to bed and so forth. If the composition of the present disclosure is a powdered, reconstitutable composition, or in a concentrated form, one serving size of powdered or concentrated composition is preferably mixed with about 50-600 ml, 100-400 ml, preferably 100-300 ml, more preferably 150-250 ml, for example about 240 ml carrier, such as water. If the composition is provided in the form of a liquid, ready-to-drink composition, one serving size corresponds to the amounts and preferred amounts of liquid and dry matter indicated above, for example, about 120 ml to about 600 ml (about 4 oz to about 20 oz), or from about 180 ml to about 360 ml (about 6 oz to about 12 oz), or from about 240 ml to about 300 ml (about 8 oz to about 10 oz). The daily serving of the composition of the present disclosure encompasses at least one serving per day. It is also possible to administer the daily administered amount in two or more servings, or three or more servings, for example. In this case, the size of the serving is preferably adjusted accordingly. Furthermore, the amount of the composition that needs to be administered in order to achieve the beneficial effects according to the present disclosure may be adjusted in dependence of the particular subject who wishes to enjoy the beneficial effects described herein. For example, one serving of the composition may be consumed in the morning, and one serving in the afternoon, the evening or before going to bed. One serving may be taken at breakfast and one serving is administered at dinner or after dinner, before going to bed. One serving of the composition of the present disclosure may be a part of or forms the breakfast of a human subject. Similarly, the preferred weight, weight percentage or weight percent of dry matter of any component or ingredient of the composition may be determined on the basis of indications of an amount of said component or ingredient per serving size, and/or daily serving, and vice versa, on the basis of the indications in this disclosure.

### BRIEF DESCRIPTION OF THE DRAWINGS

FIG. 1 shows an example of task assignment used for the evaluation of acute effects of example beverages on cognitive function and alertness in healthy adults function according to Example 1.

FIG. 2A shows comparative results of fatigue by Profile of Mood Scale (POMS) measurement for various example beverages according to study 1 of Example 1.

FIG. 2B shows comparative results of tension by POMS measurement for various example beverages according to study 1 of Example 1.

FIG. 2C shows comparative results of Bond-Lader calmness measurement for various example beverages according to study 1 of Example 1.

FIG. 2D shows comparative results of Stroop reaction time measurement for various example beverages according to study 1 of Example 1.

FIG. 2E shows comparative results of 2-back reaction time measurement for various example beverages according to study 1 of Example 1.

FIG. 3A shows comparative results of fatigue by Profile of Mood Scale (POMS) measurement for various example beverages according to study 2 of Example 1.

FIG. 3B shows comparative results of Bond-Lader alert measurement for various example beverages according to study 2 of Example 1.

FIG. 3C shows comparative results of Bond-Lader content measurement for various example beverages according to study 2 of Example 1.

FIG. 3D shows comparative results of 2-back reaction time measurement for various example beverages according to study 2 of Example 1.

FIG. 4A shows comparative results working memory accuracy measurement for various example beverages according to study 3 of Example 1.

FIG. 4B shows comparative results of 2-back reaction time measurement for various example beverages according to study 3 of Example 1.

FIG. 4C shows comparative results of Stroop reaction time measurement for various example beverages according to study 3 of Example 1.

### DETAILED DESCRIPTION

### Compositions and Ingredients

In one aspect, the present disclosure relates to an edible composition comprising: a first polyphenol; and at least one essential oil(s), wherein a ratio of the total polyphenol to the total essential oil(s) is from about 1:10 to about 100:1. In some embodiments, the polyphenol and the at least one essential oil(s) are derived from a natural resource as such fruit or vegetables. In some embodiments, the present composition is free or substantially free from caffeine.

The present disclosure is based, at least in part, on the surprising findings that administration of edible compositions or beverages having both a polyphenol and at least one essential oil(s) with a weight ratio of the polyphenol to the at least one essential oil(s) from about 1:10 to about 100:1 are effective in improving one or more of the following conditions or functions: improving cognitive function; reducing mental fatigue, improving alertness, reducing stress, improving tranquility, improving alertness, improving mental capability, improving memory capability, improving memory accuracy, improving mood, improving relaxation, reducing headache, improving/sustaining attention, improving sports performance, improving sleep functions, promoting maintenance of normal sleep, reducing sleep onset latency, improving sleep quality, alleviating of the subjective feeling of jet leg, or.

Results from a randomized, double-blind, placebo-controlled, balanced cross-over trial study (See Example 1) indicated that the present compositions are significantly better than controlled beverages in various cognition/alertness tests on healthy humans.

### Polyphenol and Essential Oil(s)

In some embodiments, the present composition comprises a polyphenol or a functional analogue, derivative, metabolites, or variation thereof; and at least one essential oil(s) or a functional analogue, derivative, metabolites, or variation thereof. Although consumption of caffeine is generally known to have positive health effects on human such as performance improvement, fatigue reduction, and decreased irritability, over consumption of caffeine may induce caffeine intoxication (caffeinism). It is for the first time identified in the present disclosure that a balanced dosage of a combination of polyphenol and at least one essential oil(s) have synergistic effect in improving the positive health benefits associated with cognitive function and/or alertness and/or mood, in the absence or with significantly lower dosage of caffeine.

In some embodiments the polyphenols disclosed herein comprise a flavonoid, a phenolic acid, a curcuminoid, a lignan, a stilbene, a phenolic terpene, a glycoside, a glucuronide, a depside or any combination thereof.

In some embodiments, a flavonoid comprises a flavanol, a flavone, a flavonol, a flavanone, an isoflavone, an anthocyanidin, a gallate, a glycoside, a polymer thereof or any combination thereof. A flavanol comprises a catechin, an epicatechin, a catechin gallate, an epicatechin gallate, a gallocatechin, an epigallocatechin, a gallocatechin gallate, an epigallocatechin gallate, a proanthocyanidin or any combinations thereof. A flavone comprises comprising a luteolin, an apigenin, a tangeritin, a chrysin, or any combination thereof. A flavonol comprises a quercetin, a kaempferol, a myricetin, a rutin, and any combination thereof. A flavanone comprises a eriodictyol, a hesperetin, a narigenin, or any combination thereof. An isoflavone comprises a genistein, a daidzein orany combination thereof. An anthocyanidin comprises a cyanidin, a delphinidin, a malvidin, a pelargonidin, an aaurantinidin, a capensinidin, an europinidin, a hirsutidin, a peonidin, a petunidin, a pulchellidin, a rosinidin and or combination thereof.

In some embodiments, the flavanol comprises a catechin, and the catechin may be in an amount of about 25 mg to about 1000 mg, about 50 mg to about 500 mg, about 100 mg to about 250 mg, about 150 mg to about 700 mg, about 200 mg to about 800 mg, and about 250 mg to about 900 mg. In some embodiments a phenolic acid comprises a rosmarinic acid, a vanillic acid, a caffeic acid, a gallic acid, a protocatechuic acid, a salicyclic acid, a ferulic acid, a sinapic acid, a chlorogenic acids, a coumaric acid or any combination thereof. In certain embodiments, chlorogenic acids are present in an amount of between about 20 mg to about 200 mg, or any range in between, or less than about 20 mg, or greater than about 200 mg.

In some embodiments a curcuminoid comprises a bisdemethoxycurcumin, a demethoxycurcumin, a curcumin, or any combination thereof.

In other embodiments a lignan comprises a lariciresinol, a pinoresino, a matairesinol, a syrigaresinol, a sesamin, a sesaminol, and any combination thereof. In other embodiments a stilbene is selected from a group comprising a resveratrol, a pterostilbene, and any combination thereof.

In some embodiments a terpene is selected from a group comprising a carnosic acid, a rosmanol, a carnosol, or/any combination thereof.

In some embodiments, the present composition has a weight ratio of the total polyphenol to the total essential oil(s) from about 1:10 to about 100:1, or from about 1:9 to about 90:1, or from about 1:8 to about 80:1, from about 1:7 to about 70:1, from about 1:6 to about 60:1, from about 1:5 to about 50:1, or from about 1:4 to about 40:1, or from about 1:3 to about 30:1, or from about 1:2 to about 20:1.

In some embodiments, the weight ratio of the total polyphenol to the total essential oil(s) is at most about 100:1, or about most about 90:1, or at most about 80:1, or at most about 70:1, or at most about 60:1, or at most about 50:1, or at most about 40:1, or at most about 30:1, or at most about 20:1, or at most 10:1.

In some embodiments, the present composition has a total content of essential oil(s) from about 0.001 wt % to about 1 wt %, or from about 0.0005 wt % to about 0.9 wt %, or from about 0.001 wt % to about 0.8 wt %, or from about 0.005 wt % to about 0.7 wt %, or from about 0.01 wt % to about 0.6 wt %, or from about 0.05 wt % to about 0.5 wt %, or from about 0.05 wt % to about 0.25 wt %, or from about 0.05 wt % to about 0.1 wt %. In some embodiments, the present composition has a total content of at least one essential oil(s) of at least about 0.001 wt %, at least about 0.002 wt %, at least about 0.003 wt %, at least about 0.004 wt %, at least about 0.005 wt %, at least about 0.006 wt %, at least about 0.007 wt %, at least about 0.008 wt %, at least about 0.009 wt %, at least about 0.01 wt %, at least about 0.02 wt %, at least about 0.03 wt %, at least about 0.04 wt %, at least about 0.05 wt %, at least about 0.06 wt %, at least about 0.07 wt %, at least about 0.08 wt %, at least about 0.09 wt %, at least 0.1 about wt %, at least about 0.2 wt %, at least about 0.3 wt %, at least about 0.4 wt %, at least about 0.5 wt %, at least about 0.6 wt %, at least about 0.7 wt %, at least about 0.8 wt %, at least about 0.9 wt %, or at least about 1 wt %. In some embodiments, the present composition has a total content of essential oil(s) of at most about 1 wt %, at most about 0.5 wt %, at most about 0.1 wt %, at most about 0.05 wt %, at most about 0.01 wt %, at most about 0.005 wt %, at most about 0.001 wt %.

In some embodiments, the present composition has a dosage of at least one essential oil(s) from about 3 mg to about 150 mg, or 10 mg to about 140 mg, or from about 50 mg to about 130 mg, from about 60 mg to about 120 mg, from about 70 mg to about 110 mg, from about 80 mg to about 100 mg, from about 85 mg to about 95 mg, or any values therebetween. In some embodiments, the present composition has a dosage of at least one essential oil(s) at least about 3 mg, at least about 4 mg, at least about 5 mg, at least about 6 mg, at least about 7 mg, at least about 8 mg, at least about 9 mg, at least about 10 mg, at least about 20 mg, at least about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, or at least about 150 mg. In some embodiments, the present composition has a dosage of at least one essential oil(s) of at most about 150 mg, at most about 100 mg, at most about 50 mg, at most about 40 mg, at most about 30 mg, at most about 20 mg, at most about 10 mg, at most about 9 mg, at most about 8 mg, at most about 7 mg, at most about 6 mg, at most about 5 mg, at most about 4 mg, at most about 3 mg.

In some embodiments the at least one essential oils(s) are selected from terpenes, monoterpenes, sesquiterpenes, diterpenes, esters, aldehydes, ketones, alcohols, phenols and oxides, and derivatives and/or any combinations thereof.

In some embodiments the first polyphenol and the least one essential oil(s) are derived from the same plant species, while in other embodiments the first polyphenol and the at least one essential oils(s) are derived from different plants species.

In additional embodiments the at least one essential oil(s) comprise a first essential oil and a second essential oil. In this embodiment the first essential oil is derived from the same or different plant species as the first polyphenol, and the second essential oil is derived from a different plant species than the first essential oil.

In non-limiting embodiments, the at least one essential oil(s) are derived from plants from the following plant genera: Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Mentha, Ocimum, Origanum, Petroselinum, Pimpinella, Piper, Thymus, Zingiber, Humulus.

In non-limiting embodiments, the at least one essential oil(s) are derived from plants from the following plant species: Rosa spp., Sophora japonica, Citrus spp., Fragaria spp., Juniperus spp., Murraya spp., Sambucus spp., Syzygium aromaticum, Angelica sinensis, Angelica acutiloba, Angelica archangelica, Helichrysum arenarium, Helichrysum italicum, Hibiscus sabdariffa, Chamomilla recutita, Chamaemelum nobile, Cymbopogon citratus, Geranium spp., Matricaria chamomilla, Jasminum sambac, Jasminum flexile, Jasminum mesnyi, Jasminum angustifolium, Lavandula spp., Melissa officinalis, Valeriana officinalis, Agave amica, Anthemis nobilis, Amomum subulatum, Cinnamomum burmanni, Cinnamomum cassia, Cinnamomum loureirin, Cinnamomum zeylanicum, Cinnamomum camphora, Coriandrum sativum, Coriandrum tordylium, Elettaria cardamomum, Eucalyptus globulus, Eucalyptus cneorifolia, Eucalyptus dives, Eucalyptus Dumosa, Eucalyptus goniocalyx, Eucalyptus horistes, Eucalyptus kochii, Eucalyptus leucoxylon, Eucalyptus largiflorens, Eucalyptus oleosa, Eucalyptus polybractea, Eucalyptus radiata, Eucalyptus rossii, Eucalyptus sideroxylon, Eucalyptus smithii, Eucalyptus. staigeriana, Eucalyptus tereticornis, Eucalyptus viridis, Capsicum spp., Curcuma longa, Euonymus phellomanus, Foeniculum vulgare, Litsea cubeba, Salvia officinalis, Salvia lavandulifolia, Salvia rosmarinus (Rosmarinus officinalis), Salvia hispanica, Salvia columbariae, Salvia polystachya, Salvia bowleyana, Salvia cavaleriei, Salvia chinensis, Salvia flava, Salvia prionitis, Salvia sonchifolia, Salvia yunnanensis, Salvia miltiorrhiza, Salvia multicaulis, Salvia divinorum, Satureia hortensis, Satureia montana, Schinus mole, Schinus terebinthifolia, Mentha piperita, Mentha spicata, Mentha citrate, Ocimum basilicum, Ocimum citriodorum, Ocimum kilimandscharicum, Ocimum Americanum, Ocimum gratissimum, Ocimum tenuiflorum, Origanum majorana, Origanum vulgare, Petroselinum crispum, Petroselimam segetu, Pimpinella anisum, Piper nigrum, Thymus vulgaris, Thymus zygis, Thymus serpyllum, Thymus citriodorus, Thymus praecox, Thymus pseudolanuginosus, Zingiber officinale, Zingiber mioga, Humulus lupulus.

In some embodiments, the present composition has a total content of polyphenol from about 0.002 wt % to about 2 wt %, or from about 0.005 wt % to about 1.5 wt %, or from about 0.01 wt % to about 1 wt %, or from about 0.05 wt % to about 0.5 wt %, or from about 0.1 wt % to about 0.2 wt %. In some embodiments, the present composition has a total content of polyphenol of at least about 0.002 wt %, at least about 0.005 wt %, at least about 0.01 wt %, at least about 0.02 wt %, at least about 0.03 wt %, at least about 0.04 wt %, at least about 0.05 wt %, at least about 0.06 wt %, at least about 0.07 wt %, at least about 0.08 wt %, at least about 0.09 wt %, at least about 0.1 wt %, at least about 0.2 wt %, at least about 0.3 wt %, at least about 0.4 wt %, at least about 0.5 wt %, at least about 0.6 wt %, at least about 0.7 wt %, at least about 0.8 wt %, at least about 0.9 wt %, at least about 1 wt %, at least about 1.1 wt %, at least about 1.2 wt %, at least about 1.3 wt %, at least about 1.4 wt %, at least about 1.5 wt %, at least about 1.6 wt %, at least about 1.7 wt %, at least about 1.8 wt %, at least about 1.9 wt %, at least about 2 wt %. In some embodiments, the present composition has a total content of polyphenol of at most about 2 wt %, at most about 1.5 wt %, at most about 1 wt %, at most about 0.5 wt %, at most about 0.1 wt %, at most about 0.05 wt %, at most about 0.01 wt %, at most about 0.005 wt %, at most about 0.002 wt %.

In some embodiments, the present composition has a dosage of polyphenol from about 15 mg to about 300 mg, or from about 20 mg to about 250 mg, or from about 30 mg to about 200 mg, from about 40 mg to about 150 mg, from about 50 mg to about 100 mg, or from about 60 mg to about 80 mg for one serving. In some embodiments, the present composition has a dosage of polyphenol of at least about 15 mg, at least about 25 mg, at least about 30 mg, at least about 40 mg, at least about 50 mg, at least about 60 mg, at least about 70 mg, at least about 80 mg, at least about 90 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, or at least about 300 mg for one serving. In some embodiments, the present composition has a dosage of polyphenol of at most about 300 mg, at most about 250 mg, at most about 200 mg, at most about 150 mg, at most about 100 mg, at most about 80 mg, at most about 60 mg, at most about, or at most 30 mg for one serving, or at most about 15 mg.

The polyphenol may be derived from a single source of plant or multiple sources of plants. In some embodiments, the present composition includes a first polyphenol derived from a first plant. In some embodiments, the present composition further includes a second polyphenol derived from a second plant, wherein the second plant is different from the first plant. The second polyphenol may be the same or different from the first polyphenol is chemical identity.

In some embodiments, the present composition comprises a first polyphenol, a second polyphenol, and at least one essential oil(s). The first polyphenol and the second polyphenol may be derived from the same or from a different plant species.

In non-limiting embodiments, the first or second polyphenols can be derived from plants from the following plant genera: Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Maginfera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.

In further non-limiting embodiments, the first or second polyphenols can be derived from plants from the following plant species. Camellia sinensis, Camellia assamica, Coffea arabica, Coffea canephora, Coffea eugenioides, Ilex paraguariensis, Ilex latifolia, Ilex vomitoria, Ilex guayusa, Ilex kudingcha, Paullinia cupana, Theobroma cacao, Hypericum perforatum, Beta vulgaris, Glycine max, Ginkgo biloba, Matricaria chamomilla, Moringa oleifera, Magnolia officinalis, Withania somnifera, Aspalathus linearis, Euterpe oleracea, Crocus sativus, Polygonum multiflorum, Polygonum cuspidatum, Ribes spp., Rosa spp., Sophora japonica, Citrus spp., Fragaria spp., Juglans spp., Litchi chinensis, Lycium barbarum, Lycium chinense, Malus spp., Mangifera indica, Murraya spp., Olea europaea, Passiflora incarnata, Prunus spp., Punica spp., Sambucus spp., Syzygium aromaticum, Vaccinium spp., Vitis spp., Aronia melanocarpa, Aronia arbutifolia, Aronia prunifolia, Angelica sinensis, Angelica acutiloba, Angelica archangelica, Hibiscus sabdariffa, Chamomilla recutita, Chamaemelum nobile, Jasminum sambac, Jasminum flexile, Jasminum mesnyi, Jasminum angustifolium, Lavandula spp., Melissa officinalis, Valeriana officinalis, Anthemis nobilis, Amomum subulatum, Cinnamomum burmanni, Cinnamomum cassia, Cinnamomum loureirin, Cinnamomum zeylanicum, Cinnamomum camphora, Coriandrum sativum, Coriandrum tordylium, Elettaria cardamomum, Eucalyptus globulus, Eucalyptus cneorifolia, Eucalyptus dives, Eucalyptus Dumosa, Eucalyptus goniocalyx, Eucalyptus horistes, Eucalyptus kochii, Eucalyptus leucoxylon, Eucalyptus largiflorens, Eucalyptus oleosa, Eucalyptus polybractea, Eucalyptus radiata, Eucalyptus rossii, Eucalyptus sideroxylon, Eucalyptus smithii, Eucalyptus. staigeriana, Eucalyptus tereticornis, Eucalyptus viridis, Capsicum spp., Curcuma longa, Foeniculum vulgare, Salvia officinalis, Salvia lavandulifolia, Salvia rosmarinus (Rosmarinus officinalis), Salvia hispanica, Salvia columbariae, Salvia polystachya, Salvia bowleyana, Salvia cavaleriei, Salvia chinensis, Salvia flava, Salvia prionitis, Salvia sonchifolia, Salvia yunnanensis, Salvia miltiorrhiza, Salvia multicaulis, Salvia divinorum, Satureia hortensis, Satureia montana, Schinus mole, Schinus terebinthifolia, Mentha piperita, Mentha spicata, Mentha citrate, Ocimum basilicum, Ocimum citriodorum, Ocimum kilimandscharicum, Ocimum Americanum, Ocimum gratissimum, Ocimum tenuiflorum, Petroselinum crispum, Petroselinum segetu, Pimpinella anisum, Piper nigrum, Thymus vulgaris, Thymus zygis, Thymus serpyllum, Thymus citriodorus, Thymus praecox, Thymus pseudolanuginosus, Zingiber officinale, Zingiber mioga, Aloe vera, Bacopa monnieri, Daucus carota, Euterpe oleracea, Humulus lupulus, Juniperus spp., Malpighia emarginata, Morus spp., Phyllanthus emblica, Rubus spp., Satureja montana.

In some embodiments, the present composition has a weight ratio of the total polyphenol to the total essential oil(s) from about 1:10 to about 100:1, or from about 1:9 to about 90:1, or from about 1:8 to about 80:1, from about 1:7 to about 70:1, from about 1:6 to about 60:1, from about 1:5 to about 50:1, or from about 1:4 to about 40:1, or from about 1:3 to about 30:1, or from about 1:2 to about 20:1.

In some embodiments, the weight ratio of the total of the first and second polyphenol to the total essential oil(s) is at most about 100:1, or about most about 90:1, or at most about 80:1, or at most about 70:1, or at most about 60:1, or at most about 50:1, or at most about 40:1, or at most about 30:1, or at most about 20:1, or at most 10:1.

In some embodiment, the present composition has a weight ratio of the first polyphenol to the second polyphenol of about 50:1 to about 1:50, from about 25:1 to about 1:25, from about 10:1 to about 1:10, from about 5:1 to about 1:5, from about 4:1 to about 1:4, from about 3:1 to about 1:3, from about 2:1 to about 1:2, or about 1:1.

### Additional/Functional Ingredients

The present oral composition can contain additional typical beverage ingredients, e.g., at least one sweetener and/or at least one functional ingredient and/or at least one additive.

### Sweetener

The present composition may optionally include a sweetener. The sweetener can be an artificial or synthetic sweetener, a natural sweetener, a natural high potency sweetener. As used herein, the phrase “natural high potency sweetener” (NHPS) refers to any sweetener found naturally in nature and characteristically has a sweetness potency greater than sucrose, fructose, or glucose, yet has less calories. The natural high potency sweetener can be provided as a pure compound or, alternatively, as part of an extract. As used herein, the phrase “synthetic sweetener” refers to any composition which is not found naturally in nature and characteristically has a sweetness potency greater than sucrose, fructose, or glucose, yet has less calories.

Non-limiting examples of NHPSs includes stevia and steviol glycosides, such as rebaudioside M, rebaudioside D, rebaudioside A, rebaudioside N, rebaudioside O, rebaudioside E, steviolmonoside, steviolbioside, rubusoside, dulcoside B, dulcoside A, rebaudioside B, rebaudioside G, stevioside, rebaudioside C, rebaudioside F, rebaudioside I, rebaudioside H, rebaudioside L, rebaudioside K, rebaudioside J, rebaudioside M2, rebaudioside D2, rebaudioside S, rebaudioside T, rebaudioside U, rebaudioside V, rebaudioside W, rebaudioside Z1, rebaudioside Z2, rebaudioside IX, enzymatically glucosylated steviol glycosides and combinations thereof. Examples of high purity steviol glycosides and methods of making the same are provided in U.S. Pat. App. No. 2021/0107933, which is hereby incorporated in its entirety.

In certain embodiments, a steviol glycoside blend comprises at least about 5% steviol glycoside by weight, such as, for example, at least about 10%, at least about 20%, at least about 30%, at least about 40%, at least about 50%, at least about 60%, at least about 70%, at least about 80%, at least about 90%, at least about 95% or at least about 97%. In exemplary embodiments, the steviol glycoside blend comprises at least about 50% steviol glycoside by weight, such as, for example, from about 50% to about 90%, from about 50% to about 80%, from about 50% to about 70%, from about 50% to about 60%, from about 60% to about 90%, from about 60% to about 80%, from about 60% to about 70%, from about 70% to about 90%, from about 70% to about 80% and from about 80% to about 90%.

Another exemplary NHPS is Luo Han Guo and the related mogroside compounds, such as grosmogroside I, mogroside IA, mogroside IE, 11-oxomogroside IA, mogroside II, mogroside II A, mogroside II B, mogroside II E, 7-oxomogroside II E, mogroside III, Mogroside HIE, 11-oxomogroside HIE, 11-deoxymogroside III, mogroside IV, Mogroside IVA 11-oxomogroside IV, 11-oxomogroside IVA, mogroside V, isomogroside V, 11-deoxymogroside V, 7-oxomogroside V, 11-oxomogroside V, isomogroside V, mogroside VI, mogrol, 11-oxomogrol, siamenoside I, isomers of siamenoside I (e.g. those disclosed in U.S. patent application No. 20170119032; incorporated by reference in its entirety), (38, 98, 10a, 11a, 24R)-3-[(4-O-β-D-glucospyranosyl-6-O-β-D-glucopyranosyl]-25-hydroxyl-9-methyl-19-norlanost-5-en-24-yl-[2-O-β-D-glucopyranosyl-6-O-β-D-glucopyranosyl]-β-D-glucopyranoside); (3B, 98, 10a, 11a, 24R)-[(2-O-β-D-glucopyranosyl-6-O-β-D-glucopyranosyl-β-D-glucopyranosyl)oxy]-25-hydroxy-9-methyl-19-norlanost-5-en-24-yl-[2-O-β-D-glucopyranosyl-6-O-β-D-glucopyranosyl]-β-D-glucopyranoside); and (3B, 9B, 10a, 11a, 24R)-[(2-O-β-D-glucopyranosyl-6-O-β-D-glucopyranosyl-β-D-glucopyranosyl)oxy]-25-hydroxy-9-methyl-19-norlanost-5-en-24-yl-[2-O-β-D-glucopyranosyl-6-O-β-D-glucopyranosyl]-β-D-glucopyranoside).

In certain embodiments, a mogroside blend comprises at least about 5% of the mogroside by weight, such as, for example, at least about 10%, at least about 20%, at least about 30%, at least about 40%, at least about 50%, at least about 60%, at least about 70%, at least about 80%, at least about 90%, at least about 95% or at least about 97%.

Other exemplary NHPSs include monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I.

In one embodiment, the sweetener is a carbohydrate sweetener. Suitable carbohydrate sweeteners include, but not limited to, the group consisting of sucrose, glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, fucose, rhamnose, arabinose, turanose, sialose and combinations thereof.

In certain embodiments, the present composition is free or substantially free from a carbohydrate sweetener.

Other suitable sweeteners include siamenoside, monatin and its salts (monatin SS, RR, RS, SR), curculin, mogrosides, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, steviolbioside and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs) and combinations thereof.

In one embodiment, the sweetener is a caloric sweetener or mixture of caloric sweeteners. In another embodiment, the caloric sweetener is selected from sucrose, fructose, glucose, high fructose com/starch syrup, a beet sugar, a cane sugar, and combinations thereof.

In certain embodiments, the present composition is free or substantially free from a caloric sweetener.

In another embodiment, the sweetener is a rare sugar selected from allulose, gulose, kojibiose, sorbose, lyxose, ribulose, xylose, xylulose, D-allose, L-ribose, D-tagatose, L-glucose, L-fucose, L-arabinose, turanose and combinations thereof.

The amount of sweetener in the present composition depends on the identity of the sweetener and the desired level of sweetness. In preferred embodiments, the sweetener is present in a sweetening amount, i.e. a concentration that is detectably sweet.

As would be understood by a person of skill in the art, high potency sweeteners are more potent and therefore lower concentrations are required to achieve a particular sucrose equivalence (SE). The sweetness of a non-sucrose sweetener can be measured against a sucrose reference by determining the non-sucrose sweetener's sucrose equivalence (SE). Typically, taste panelists are trained to detect sweetness of reference sucrose solutions containing between 1-15% sucrose (w/v). Other non-sucrose sweeteners are then tasted at a series of dilutions to determine the concentration of the non-sucrose sweetener that is as sweet as a given percent sucrose reference. For example, if a 1% solution of a non-sucrose sweetener is as sweet as a 10% sucrose solution, then the sweetener is said to be 10 times as potent as sucrose and has 10% sucrose equivalence.

In one embodiment, the sweetener or sweeteners provides the present composition with a sucrose equivalence of about 1 wt %, such as, for example, about 2%, about 3%, about 4%, about 5%, about 6%, about 7%, about 8%, about 9%, about 10%, about 11%, about 12%, about 13%, about 14% or any range between these values.

In another embodiment, the present composition has a SE from about 2% to about 14%, such as, for example, from about 2% to about 10%, from about 2% to about 5%, from about 5% to about 15%, from about 5% to about 10% or from about 10% to about 15%.

The amount of sucrose, and thus another measure of sweetness, in a reference solution may be described in degrees Brix (° Bx). One degree Brix is 1 gram of sucrose in 100 grams of solution and represents the strength of the solution as percentage by weight (% w/w) (strictly speaking, by mass). In embodiments where the present composition is sweetened with a sweetener, the composition has a Brix value of about 1° to about 25°, or about 5° to about 20°, or about 7° to about 15°. In some embodiments, the composition can be at least about 1° Bx, about 2° Bx, about 3° Bx, about 4° Bx, about 5° Bx, about 6° Bx, about 7° Bx, about 8° Bx, about 9° Bx, about 10° Bx, about 11° Bx, about 12° Bx, about 13° Bx, about 14° Bx, about 15° Bx, about 16° Bx, about 17° Bx, about 18° Bx, about 19° Bx, about 20° Bx, about 21° Bx, about 22° Bx, about 23° Bx, about 24° Bx, about 25° Bx, or any range between these values.

The present composition may optionally include a functional ingredient. Exemplary functional ingredients include, but are not limited to, electrolytes, saponins, antioxidants, dietary fiber sources, fatty acids, vitamins, glucosamine, minerals, preservatives, hydration agents, probiotics, prebiotics, weight management agents, osteoporosis management agents, terpenes or derivatives thereof, phytoestrogens, long chain primary aliphatic saturated alcohols, phytosterols and combinations thereof.

### Antioxidant

In certain embodiments, the functional ingredient is at least one antioxidant. As used herein, “antioxidant” refers to any substance which inhibits, suppresses, or reduces oxidative damage to cells and biomolecules.

Examples of suitable antioxidants for embodiments of this disclosure include, but are not limited to, vitamins, vitamin cofactors, minerals, hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, and combinations thereof. In some embodiments, the antioxidant is vitamin A, vitamin C, vitamin E, ubiquinone, mineral selenium, manganese, melatonin, oc-carotene, b-carotene, lycopene, lutein, zeanthin, crypoxanthin, reservatol, eugenol, quercetin, catechin, gossypol, hesperetin, curcumin, ferulic acid, thymol, hydroxytyrosol, tumeric, thyme, olive oil, lipoic acid, glutathinone, gutamine, oxalic acid, tocopherol-derived compounds, butylated hydroxyanisole (BHA), butylated hydroxytoluene (BHT), ethylenediaminetetraacetic acid (EDTA), tert-butylhydroquinone, acetic acid, pectin, tocotrienol, tocopherol, coenzyme Q10, zeaxanthin, astaxanthin, canthaxantin, saponins, limonoids, kaempfedrol, myricetin, isorhamnetin, proanthocyanidins, quercetin, rutin, luteolin, apigenin, tangeritin, hesperetin, naringenin, erodictyol, flavan-3-ols (e.g., anthocyanidins), gallocatechins, epicatechin and its gallate forms, epigallocatechin and its gallate forms (ECGC) theaflavin and its gallate forms, thearubigins, isoflavone, phytoestrogens, genistein, daidzein, glycitein, anythocyanins, cyaniding, delphinidin, malvidin, pelargonidin, peonidin, petunidin, ellagic acid, gallic acid, salicylic acid, rosmarinic acid, cinnamic acid and its derivatives (e.g., ferulic acid), chlorogenic acid, chicoric acid, gallotannins, ellagitannins, anthoxanthins, betacyanins and other plant pigments, silymarin, citric acid, lignan, antinutrients, bilirubin, uric acid, R-oc-lipoic acid, N-acetylcysteine, emblicanin, apple extract, apple skin extract (applephenon), rooibos extract red, rooibos extract, green, hawthorn berry extract, red raspberry extract, green coffee antioxidant (GCA), aronia extract 20%, grape seed extract (i.e. VINOSEED), cocoa extract, hops extract, mangosteen extract, mangosteen hull extract, cranberry extract, pomegranate extract, pomegranate hull extract, pomegranate seed extract, hawthorn berry extract, pomella pomegranate extract, cinnamon bark extract, grape skin extract, bilberry extract, pine bark extract, pycnogenol, elderberry extract, mulberry root extract, wolfberry (gogi) extract, blackberry extract, blueberry extract, blueberry leaf extract, raspberry extract, turmeric extract, citrus bioflavonoids, black currant, ginger, acai powder, green coffee bean extract, green tea extract, and phytic acid, or combinations thereof. In alternate embodiments, the antioxidant is a synthetic antioxidant such as butylated hydroxytolune or butylated hydroxyanisole, for example. Other sources of suitable antioxidants for embodiments of this disclosure include, but are not limited to, fruits, vegetables, tea, cocoa, chocolate, spices, herbs, rice, organ meats from livestock, yeast, whole grains, or cereal grains.

It is noted that the polyphenol, phenolic acid, or chlorogenic acid contained in the present compositions can also serve as antioxidants. Other suitable polyphenols for embodiments of this disclosure, as mentioned supra, can include catechins, proanthocyanidins, procyanidins, anthocyanins, quercerin, rutin, reservatrol, isoflavones, curcumin, punicalagin, ellagitannin, hesperidin, naringin, citrus flavonoids, other similar materials, and combinations thereof. In one embodiment, the present composition includes antioxidant comprising a catechin such as, for example, epigallocatechin gallate (EGCG).

### Dietary Fiber

In certain embodiments, the functional ingredient is at least one dietary fiber. Numerous polymeric carbohydrates having significantly different structures in both composition and linkages fall within the definition of dietary fiber. Such compounds are well known to those skilled in the art, non-limiting examples of which include non-starch polysaccharides, lignin, cellulose, methylcellulose, the hemicelluloses, b-glucans, pectins, gums, mucilage, waxes, inulins, oligosaccharides, fructooligosaccharides, cyclodextrins, chitins, and combinations thereof. Although dietary fiber generally is derived from plant sources, indigestible animal products such as chitins are also classified as dietary fiber. Chitin is a polysaccharide composed of units of acetylglucosamine joined by β (1-4) linkages, similar to the linkages of cellulose.

### Fatty Acid

In certain embodiments, the functional ingredient is at least one fatty acid. As used herein, “fatty acid” refers to any straight chain monocarboxylic acid and includes saturated fatty acids, unsaturated fatty acids, long chain fatty acids, medium chain fatty acids, short chain fatty acids, fatty acid precursors (including omega-9 fatty acid precursors), and esterified fatty acids. As used herein, “long chain polyunsaturated fatty acid” refers to any polyunsaturated carboxylic acid or organic acid with a long aliphatic tail. As used herein, “omega-3 fatty acid” refers to any polyunsaturated fatty acid having a first double bond as the third carbon-carbon bond from the terminal methyl end of its carbon chain. In particular embodiments, the omega-3 fatty acid may comprise a long chain omega-3 fatty acid. As used herein, “omega-6 fatty acid” any polyunsaturated fatty acid having a first double bond as the sixth carbon-carbon bond from the terminal methyl end of its carbon chain.

Suitable omega-3 fatty acids for use in embodiments of the present disclosure can be derived from algae, fish, animals, plants, or combinations thereof, for example. Examples of suitable omega-3 fatty acids include, but are not limited to, linolenic acid, alpha-linolenic acid, eicosapentaenoic acid, docosahexaenoic acid, stearidonic acid, eicosatetraenoic acid and combinations thereof. In some embodiments, suitable omega-3 fatty acids can be provided in fish oils, (e.g., menhaden oil, tuna oil, salmon oil, bonito oil, and cod oil), microalgae omega-3 oils or combinations thereof. In particular embodiments, suitable omega-3 fatty acids may be derived from commercially available omega-3 fatty acid oils such as Microalgae DHA oil (from Martek, Columbia, MD), OmegaPure (from Omega Protein, Houston, TX), Marinol C-38 (from Lipid Nutrition, Channahon, IL), Bonito oil and MEG-3 (from Ocean Nutrition, Dartmouth, NS), Evogel (from Symrise, Holzminden, Germany), Marine Oil, from tuna or salmon (from Arista Wilton, CT), OmegaSource 2000, Marine Oil, from menhaden and Marine Oil, from cod (from OmegaSource, RTP, NC). Suitable omega-6 fatty acids include, but are not limited to, linoleic acid, gamma-linolenic acid, dihommo-gamma-linolenic acid, arachidonic acid, eicosadienoic acid, docosadienoic acid, adrenic acid, docosapentaenoic acid and combinations thereof.

Suitable esterified fatty acids for embodiments of the present disclosure include, but are not limited to, monoacylgycerols containing omega-3 and/or omega-6 fatty acids, diacylgycerols containing omega-3 and/or omega-6 fatty acids, or triacylgycerols containing omega-3 and/or omega-6 fatty acids and combinations thereof.

### Vitamin

In certain embodiments, the functional ingredient is at least one vitamin. Suitable vitamins include, vitamin A, vitamin D, vitamin E, vitamin K, vitamin B1, vitamin B2, vitamin B3, vitamin B5, vitamin B6, vitamin B7, vitamin B9, vitamin B 12, and vitamin C.

Various other compounds have been classified as vitamins by some authorities. These compounds may be termed pseudo-vitamins and include, but are not limited to, compounds such as ubiquinone (coenzyme Q10), pangamic acid, dimethylglycine, taestrile, amygdaline, flavanoids, para-aminobenzoic acid, adenine, adenylic acid, and s-methylmethionine. As used herein, the term vitamin includes pseudo-vitamins. In some embodiments, the vitamin is a fat-soluble vitamin chosen from vitamin A, D, E, K and combinations thereof. In other embodiments, the vitamin is a water-soluble vitamin chosen from vitamin B1, vitamin B2, vitamin B3, vitamin B6, vitamin B12, folic acid, biotin, pantothenic acid, vitamin C and combinations thereof.

### Mineral

In certain embodiments, the functional ingredient is at least one mineral. Minerals, in accordance with the teachings of this disclosure, comprise inorganic chemical elements required by living organisms. Minerals are comprised of a broad range of compositions (e.g., elements, simple salts, and complex silicates) and also vary broadly in crystalline structure. They may naturally occur in foods and beverages, may be added as a supplement, or may be consumed or administered separately from foods or beverages.

Minerals may be categorized as either bulk minerals, which are required in relatively large amounts, or trace minerals, which are required in relatively small amounts. Bulk minerals generally are required in amounts greater than or equal to about 100 mg per day and trace minerals are those that are required in amounts less than about 100 mg per day. In one embodiment, the mineral is chosen from bulk minerals, trace minerals or combinations thereof. Non-limiting examples of bulk minerals include calcium, chlorine, magnesium, phosphorous, potassium, sodium, and sulfur. Non-limiting examples of trace minerals include chromium, cobalt, copper, fluorine, iron, manganese, molybdenum, selenium, zinc, and iodine. Although iodine generally is classified as a trace mineral, it is required in larger quantities than other trace minerals and often is categorized as a bulk mineral.

In a particular embodiment, the mineral is a trace mineral, believed to be necessary for human nutrition, non-limiting examples of which include bismuth, boron, lithium, nickel, rubidium, silicon, strontium, tellurium, tin, titanium, tungsten, and vanadium.

The minerals embodied herein may be in any form known to those of ordinary skill in the art. For example, in one embodiment, the minerals may be in their ionic form, having either a positive or negative charge. In another embodiment, the minerals may be in their molecular form. For example, sulfur and phosphorous often are found naturally as sulfates, sulfides, and phosphates.

### Preservative

In certain embodiments, the functional ingredient is at least one preservative. In particular embodiments, the preservative is chosen from antimicrobials, antioxidants, antienzymatics or combinations thereof. Non-limiting examples of antimicrobials include sulfites, propionates, benzoates, sorbates, nitrates, nitrites, bacteriocins, salts, sugars, acetic acid, dimethyl dicarbonate (DMDC), ethanol, and ozone. In one embodiment, the preservative is a sulfite. Sulfites include, but are not limited to, sulfur dioxide, sodium bisulfite, and potassium hydrogen sulfite. In another embodiment, the preservative is a propionate. Propionates include, but are not limited to, propionic acid, calcium propionate, and sodium propionate. In yet another embodiment, the preservative is a benzoate. Benzoates include, but are not limited to, sodium benzoate and benzoic acid. In still another embodiment, the preservative is a sorbate. Sorbates include, but are not limited to, potassium sorbate, sodium sorbate, calcium sorbate, and sorbic acid. In a still further embodiment, the preservative is a nitrate and/or a nitrite. Nitrates and nitrites include, but are not limited to, sodium nitrate and sodium nitrite. In another embodiment, the at least one preservative is a bacteriocin, such as, for example, nisin. In still another embodiment, the preservative is ethanol. In yet another embodiment, the preservative is ozone. Non-limiting examples of anti-enzymatis suitable for use as preservatives in particular embodiments of the disclosure include ascorbic acid, citric acid, and metal chelating agents such as ethylenediaminetetraacetic acid (EDTA). In some embodiments, the present composition is free or substantially free from a preservative.

### Probiotic

In certain embodiments, the functional ingredient is chosen from at least one probiotic, prebiotic and combination thereof. The probiotic is a beneficial microorganism that affects the human body's naturally occurring gastrointestinal microflora. Examples of probiotics include, but are not limited to, bacteria of the genus Lactobacilli, Bifidobacteria, Streptococci, or combinations thereof, that confer beneficial effects to humans. In particular embodiments of the disclosure, the at least one probiotic is chosen from the genus Lactobacilli. According to other particular embodiments of this disclosure, the probiotic is chosen from the genus Bifidobacteria. In a particular embodiment, the probiotic is chosen from the genus Streptococcus.

Probiotics that may be used in accordance with this disclosure are well-known to those of skill in the art. Non-limiting examples of foodstuffs comprising probiotics include yogurt, sauerkraut, kefir, kimchi, fermented vegetables, and other foodstuffs containing a microbial element that beneficially affects the host animal by improving the intestinal microbalance.

Prebiotics, in accordance with the embodiments of this disclosure, include, without limitation, mucopolysaccharides, oligosaccharides, polysaccharides, amino acids, vitamins, nutrient precursors, proteins and combinations thereof. According to a particular embodiment of this disclosure, the prebiotic is chosen from dietary fibers, including, without limitation, polysaccharides and oligosaccharides. Non-limiting examples of oligosaccharides that are categorized as prebiotics in accordance with particular embodiments of this disclosure include fructooligosaccharides, inulins, isomalto-oligosaccharides, lactilol, lactosucrose, lactulose, pyrodextrins, soy oligosaccharides, transgalacto-oligosaccharides, and xylo-oligosaccharides. In other embodiments, the prebiotic is an amino acid. Although a number of known prebiotics break down to provide carbohydrates for probiotics, some probiotics also require amino acids for nourishment.

In some embodiments, the amino acid may be in an amount of about 5 mg/g to about 50 mg/g, about 10 mg/g to about 100 mg/g, or about 13.7 mg/g to about 23.8 mg/g.

In some embodiments, the present composition is free or substantially free from probiotics.

### Prebiotics

Prebiotics are found naturally in a variety of foods including, without limitation, bananas, berries, asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans). In some embodiments, the present composition is free or substantially free from prebiotics.

### Weight Management Agent

In certain embodiments, the functional ingredient is at least one weight management agent. As used herein, “a weight management agent” includes an appetite suppressant and/or a thermogenesis agent. As used herein, the phrases “appetite suppressant”, “appetite satiation compositions”, “satiety agents”, and “satiety ingredients” are synonymous. The phrase “appetite suppressant” describes macronutrients, herbal extracts, exogenous hormones, anorectics, anorexigenics, pharmaceutical drugs, and combinations thereof, that when delivered in an effective amount, suppress, inhibit, reduce, or otherwise curtail a person's appetite. The phrase “thermogenesis agent” describes macronutrients, herbal extracts, exogenous hormones, anorectics, anorexigenics, pharmaceutical drugs, and combinations thereof, that when delivered in an effective amount, activate or otherwise enhance a person's thermogenesis or metabolism.

Suitable weight management agents include macronutrients selected from the group consisting of proteins, carbohydrates, dietary fats, and combinations thereof. Consumption of proteins, carbohydrates, and dietary fats stimulates the release of peptides with appetite suppressing effects. For example, consumption of proteins and dietary fats stimulates the release of the gut hormone cholecytokinin (CCK), while consumption of carbohydrates and dietary fats stimulates release of Glucagon-like peptide 1 (GLP-1).

Suitable macronutrient weight management agents also include carbohydrates. Carbohydrates generally comprise sugars, starches, cellulose and gums that the body converts into glucose for energy. Carbohydrates often are classified into two categories, digestible carbohydrates (e.g., monosaccharides, disaccharides, and starch) and non-digestible carbohydrates (e.g., dietary fiber). Studies have shown that non-digestible carbohydrates and complex polymeric carbohydrates having reduced absorption and digestibility in the small intestine stimulate physiologic responses that inhibit food intake. Accordingly, the carbohydrates embodied herein desirably comprise non-digestible carbohydrates or carbohydrates with reduced digestibility. Non-limiting examples of such carbohydrates include polydextrose; inulin; monosaccharide-derived polyols such as erythritol, mannitol, xylitol, and sorbitol; disaccharide-derived alcohols such as isomalt, lactitol, and maltitol; and hydrogenated starch hydrolysates. Carbohydrates are described in more detail herein below.

### Dietary Fat

In another particular embodiment, the weight management agent is a dietary fat. Dietary fats are lipids comprising combinations of saturated and unsaturated fatty acids. Polyunsaturated fatty acids have been shown to have a greater satiating power than mono-unsaturated fatty acids. Accordingly, the dietary fats embodied herein desirably comprise poly-unsaturated fatty acids, non-limiting examples of which include triacylglycerols. In another particular embodiment, the weight management agent is an herbal extract. Extracts from numerous types of plants have been identified as possessing appetite suppressant properties. Non-limiting examples of plants whose extracts have appetite suppressant properties include plants of the genus Hoodia, Trichocaulon, Caralluma, Stapelia, Orbea, Asclepias, and Camelia. Other embodiments include extracts derived from Gymnema Sylvestre, Kola Nut, Citrus Auran tium, Yerba Mate, Griff onia Simplicifolia, Guarana, myrrh, guggul Lipid, and black current seed oil.

In another embodiment, the weight management agent is a pharmaceutical drug. Non limiting examples include phentenime, diethylpropion, phendimetrazine, sibutramine, rimonabant, oxyntomodulin, floxetine hydrochloride, ephedrine, phenethylamine, or other stimulants.

### Herbal Extracts

The herbal extracts may be prepared from any type of plant material or plant biomass. Non-limiting examples of plant material and biomass include the stems, roots, leaves, dried powder obtained from the plant material, and sap or dried sap. The herbal extracts generally are prepared by extracting sap from the plant and then spray-drying the sap. Alternatively, solvent extraction procedures may be employed. Following the initial extraction, it may be desirable to further fractionate the initial extract (e.g., by column chromatography) in order to obtain an herbal extract with enhanced activity. Such techniques are well known to those of ordinary skill in the art.

In one embodiment, the herbal extract is derived from a plant of the genus Hoodia. A sterol glycoside of Hoodia, known as P57, is believed to be responsible for the appetite-suppressant effect of the Hoodia species. In another embodiment, the herbal extract is derived from a plant of the genus Caralluma, non-limiting examples of which include caratuberside A, caratuberside B, bouceroside I, bouceroside II, bouceroside III, bouceroside IV, bouceroside V, bouceroside VI, bouceroside VII, bouceroside VIII, bouceroside IX, and bouceroside X. In another embodiment, the at least one herbal extract is derived from a plant of the genus Trichocaulon. Trichocaulon plants are succulents that generally are native to southern Africa, similar to Hoodia, and include the species T piliferum and T officinale. In another embodiment, the herbal extract is derived from a plant of the genus Stapelia or Orbea. Not wishing to be bound by any theory, it is believed that the compounds exhibiting appetite suppressant activity are saponins, such as pregnane glycosides, which include stavarosides A, B, C, D, E, F, G, H, I, J, and K. In another embodiment, the herbal extract is derived from a plant of the genus Asclepias. Not wishing to be bound by any theory, it is believed that the extracts comprise steroidal compounds, such as pregnane glycosides and pregnane aglycone, having appetite suppressant effects. In another particular embodiment, the weight management agent is an exogenous hormone having a weight management effect. Non-limiting examples of such hormones include CCK, peptide YY, ghrelin, bombesin and gastrin-releasing peptide (GRP), enterostatin, apolipoprotein A-IV, GLP-1, amylin, somastatin, and leptin.

### Osteoporosis Management Agent

In certain embodiments, the functional ingredient is at least one osteoporosis management agent. In certain embodiments, the osteoporosis management agent is at least one calcium source. According to a particular embodiment, the calcium source is any compound containing calcium, including salt complexes, solubilized species, and other forms of calcium. Non-limiting examples of calcium sources include amino acid chelated calcium, calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium citrate malate, calcium gluconate, calcium tartrate, calcium lactate, solubilized species thereof, and combinations thereof.

According to a particular embodiment, the osteoporosis management agent is a magnesium source. The magnesium source is any compound containing magnesium, including salt complexes, solubilized species, and other forms of magnesium. Non-limiting examples of magnesium sources include magnesium chloride, magnesium citrate, magnesium gluceptate, magnesium gluconate, magnesium lactate, magnesium hydroxide, magnesium picolate, magnesium sulfate, solubilized species thereof, and mixtures thereof. In another particular embodiment, the magnesium source comprises an amino acid chelated or amino acid formulation chelated magnesium.

In other embodiments, the osteoporosis agent is chosen from vitamins D, C, K, their precursors and/or beta-carotene and combinations thereof.

Numerous plants and plant extracts also have been identified as being effective in the prevention and treatment of osteoporosis. Non-limiting examples of suitable plants and plant extracts as osteoporosis management agents include species of the genus Taraxacum and Amelanchier, as disclosed in U.S. Patent Publication No. 2005/0106215, and species of the genus Lindera, Artemisia, Acorus, Carthamus, Carum, Cnidium, Curcuma, Cyperus, Juniperus, Prunus, Iris, Cichorium, Dodonaea, Epimedium, Erigonoum, rioya, Mentha, Ocimum, thymus, Tanacetum, Plantago, Spearmint, Bixa, Vitis, Rosemarinus, Rhus, and Anethum, as disclosed in U.S. Pat. App. No. 2005/0079232.

### Phytoestrogen

In certain embodiments, the functional ingredient is at least one phytoestrogen. Phytoestrogens are compounds found in plants which can typically be delivered into human bodies by ingestion of the plants or the plant parts having the phytoestrogens. As used herein, “phytoestrogen” refers to any substance which, when introduced into a body causes an estrogen like effect of any degree. For example, a phytoestrogen may bind to estrogen receptors within the body and have a small estrogen-like effect.

Examples of suitable phytoestrogens for embodiments of this disclosure include, but are not limited to, isoflavones, stilbenes, lignans, resorcyclic acid lactones, coumestans, coumestrol, equol, and combinations thereof. Sources of suitable phytoestrogens include, but are not limited to, whole grains, cereals, fibers, fruits, vegetables, black cohosh, agave root, black currant, black haw, chasteberries, cramp bark, dong quai root, devil's club root, false unicorn root, ginseng root, groundsel herb, licorice, liferoot herb, motherwort herb, peony root, raspberry leaves, rose family plants, sage leaves, sarsaparilla root, saw palmetto berried, wild yam root, yarrow blossoms, legumes, soybeans, soy products (e.g., miso, soy flour, soymilk, soy nuts, soy protein isolate, tempen, or tofu) chick peas, nuts, lentils, seeds, clover, red clover, dandelion leaves, dandelion roots, fenugreek seeds, green tea, hops, red wine, flaxseed, garlic, onions, linseed, borage, butterfly weed, caraway, chaste tree, vitex, dates, dill, fennel seed, gotu kola, milk thistle, pennyroyal, pomegranates, southernwood, soya flour, tansy, and root of the kudzu vine (pueraria root) and the like, and combinations thereof.

Isoflavones belong to the group of polyphenols as described supra. Suitable phytoestrogen isoflavones in accordance with embodiments of this disclosure include genistein, daidzein, glycitein, biochanin A, formononetin, their respective naturally occurring glycosides and glycoside conjugates, matairesinol, secoisolariciresinol, enter olactone, enterodiol, textured vegetable protein, and combinations thereof. Suitable sources of isoflavones for embodiments of this disclosure include, but are not limited to, soybeans, soy products, legumes, alfalfa sprouts, chickpeas, peanuts, and red clover.

### Long Chain Primary Aliphatic Saturated Alcohol

In certain embodiments, the functional ingredient is at least one long chain primary aliphatic saturated alcohol. Long-chain primary aliphatic saturated alcohols are a diverse group of organic compounds. The term alcohol refers to the fact these compounds feature a hydroxyl group (—OH) bound to a carbon atom. Non-limiting examples of particular long-chain primary aliphatic saturated alcohols for use in particular embodiments of the disclosure include the 8 carbon atom 1-octanol, the 9 carbon 1-nonanol, the 10 carbon atom 1-decanol, the 12 carbon atom 1-dodecanol, the 14 carbon atom 1-tetradecanol, the 16 carbon atom 1-hexadecanol, the 18 carbon atom 1-octadecanol, the 20 carbon atom 1-eicosanol, the 22 carbon 1-docosanol, the 24 carbon 1-tetracosanol, the 26 carbon 1-hexacosanol, the 27 carbon 1-heptacosanol, the 28 carbon 1-octanosol, the 29 carbon 1-nonacosanol, the 30 carbon 1-triacontanol, the 32 carbon 1-dotriacontanol, and the 34 carbon 1-tetracontanol.

In one embodiment, the long-chain primary aliphatic saturated alcohol is a policosanol. Policosanol is the term for a mixture of long-chain primary aliphatic saturated alcohols composed primarily of 28 carbon 1-octanosol and 30 carbon 1-triacontanol, as well as other alcohols in lower concentrations such as 22 carbon 1-docosanol, 24 carbon 1-tetracosanol, 26 carbon 1-hexacosanol, 27 carbon 1-heptacosanol, 29 carbon 1-nonacosanol, 32 carbon 1-dotriacontanol, and 34 carbon 1-tetracontanol.

In certain embodiments, the functional ingredient is at least one phytosterol, phytostanol or combination thereof. As used herein, the phrases “stand”, “plant stand” and “phytostanol” are synonymous. Plant sterols and stands are present naturally in small quantities in many fruits, vegetables, nuts, seeds, cereals, legumes, vegetable oils, bark of the trees and other plant sources. Sterols are a subgroup of steroids with a hydroxyl group at C-3. Generally, phytosterols have a double bond within the steroid nucleus, like cholesterol; however, phytosterols also may comprise a substituted side chain (R) at C-24, such as an ethyl or methyl group, or an additional double bond. The structures of phytosterols are well known to those of skill in the art.

At least 44 naturally occurring phytosterols have been discovered, and generally are derived from plants, such as corn, soy, wheat, and wood oils; however, they also may be produced synthetically to form compositions identical to those in nature or having properties similar to those of naturally occurring phytosterols. Non-limiting suitable phytosterols include, but are not limited to, 4-desmethylsterols (e.g., b-sitosterol, campesterol, stigmasterol, brassicasterol, 22-dehydrobrassicasterol, and A5-avenasterol), 4-monomethyl sterols, and 4,4-dimethyl sterols (triterpene alcohols) (e.g., cycloartenol, 24-methylenecycloartanol, and cyclobranol).

As used herein, the phrases “stanol”, “plant stand” and “phytostanol” are synonymous. Phytostanols are saturated sterol alcohols present in only trace amounts in nature and also may be synthetically produced, such as by hydrogenation of phytosterols. Suitable phytostanols include, but are not limited to, b-sitostanol, campestanol, cycloartanol, and saturated forms of other triterpene alcohols.

Both phytosterols and phytostanols, as used herein, include the various isomers such as the a and b isomers. The phytosterols and phytostanols of the present disclosure also may be in their ester form. Suitable methods for deriving the esters of phytosterols and phytostanols are well known to those of ordinary skill in the art, and are disclosed in U.S. Pat. Nos. 6,589,588, 6,635,774, 6,800,317, and U.S. Pat. App. No. 2003/0045473. Non limiting examples of suitable phytosterol and phytostanol esters include sitosterol acetate, sitosterol oleate, stigmasterol oleate, and their corresponding phytostanol esters. The phytosterols and phytostanols of the present disclosure also may include their derivatives.

### Other Additives

Exemplary additives include, but not limited to, carbohydrates, polyols, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, plant extracts, flavonoids, alcohols, polymers and combinations thereof.

In some embodiments, the composition comprises less than 200 mg caffeine, less than 175 mg caffeine, less than 150 mg caffeine, less than 125 mg caffeine, less than 100 mg caffeine, less than 75 mg caffeine, less than 50 mg caffeine, less than 25 mg caffeine, less than 10 mg caffeine, but more than 0 mg caffeine. In some embodiments, the caffeine content may be 0 mg.

In one embodiment, the composition further comprises one or more polyols. The term “polyol”, as used herein, refers to a molecule that contains more than one hydroxyl group. A polyol may be a diol, triol, or a tetrad which contains 2, 3, and 4 hydroxyl groups respectively.

A polyol also may contain more than 4 hydroxyl groups, such as a pentad, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Non-limiting examples of polyols in some embodiments include maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, and sugar alcohols or any other carbohydrates capable of being reduced which do not adversely affect taste.

Suitable sugar acid additives include, but are not limited to, aldonic, uronic, aldaric, alginic, gluconic, glucuronic, glucaric, galactaric, galacturonic, and salts thereof (e.g., sodium, potassium, calcium, magnesium salts or other physiologically acceptable salts), and combinations thereof.

Suitable nucleotide additives include, but are not limited to, inosine monophosphate (IMP), guanosine monophosphate (GMP), adenosine monophosphate (AMP), cytosine monophosphate (CMP), uracil monophosphate (UMP), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g., guanine, cytosine, adenine, thymine, uracil).

Suitable organic acid additives include any compound which comprises a —COOH moiety, such as, for example, C2-C30 carboxylic acids, substituted hydroxyl C2-C30 carboxylic acids, butyric acid (ethyl esters), substituted butyric acid (ethyl esters), benzoic acid, substituted benzoic acids (e.g, 2,4-dihydroxybenzoic acid), substituted cinnamic acids, hydroxyacids, substituted hydroxybenzoic acids, anisic acid substituted cyclohexyl carboxylic acids, tannic acid, aconitic acid, lactic acid, tartaric acid, citric acid, isocitric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, amino acid formulation, caffeic acid, bile acids, acetic acid, ascorbic acid, alginic acid, erythorbic acid, polyglutamic acid, glucono delta lactone, and their alkali or alkaline earth metal salt derivatives thereof. In addition, the organic acid additives also may be in either the D- or L-configuration. Suitable organic acid additive salts include, but are not limited to, sodium, calcium, potassium, and magnesium salts of all organic acids, such as salts of citric acid, malic acid, tartaric acid, fumaric acid, lactic acid (e.g., sodium lactate), alginic acid (e.g., sodium alginate), ascorbic acid (e.g., sodium ascorbate), benzoic acid (e.g., sodium benzoate or potassium benzoate), sorbic acid and adipic acid. The examples of the organic acid additives described optionally may be substituted with at least one group chosen from hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfmyl, sulfamyl, carboxalkoxy, carboxamido, phosphonyl, phosphinyl, phosphoryl, phosphino, thioester, thioether, anhydride, oximino, hydrazino, carbamyl, phosphor or phosphonato. In particular embodiments, the organic acid additive is present in the sweetener composition in an amount effective to provide a concentration from about 10 ppm to about 5,000 ppm when present in a consumable, such as, for example, a beverage.

Suitable inorganic acid additives include, but are not limited to, phosphoric acid, phosphorous acid, polyphosphoric acid, hydrochloric acid, sulfuric acid, carbonic acid, sodium dihydrogen phosphate, and alkali or alkaline earth metal salts thereof (e.g., inositol hexaphosphate Mg/Ca).

Suitable bitter compound additives include, but are not limited to, caffeine, quinine, urea, bitter orange oil, naringin, quassia, and salts thereof.

Suitable flavorants and flavoring ingredient additives include, but are not limited to, vanillin, vanilla extract, mango extract, cinnamon, citrus, coconut, ginger, viridiflorol, almond, menthol (including menthol without mint), grape skin extract, and grape seed extract. “Flavorant” and “flavoring ingredient” are synonymous and can include natural or synthetic substances or combinations thereof. Flavorants also include any other substance which imparts flavor and may include natural or non-natural (synthetic) substances which are safe for human or animals when used in a generally accepted range. Non-limiting examples of proprietary flavorants include DOHLER™ Natural Flavoring Sweetness Enhancer K14323 (DOHLER™, Darmstadt, Germany), Symrise™ Natural Flavor Mask for Sweeteners 161453 and 164126 (SYMRISE™, Holzminden, Germany), Natural Advantage™ Bitterness Blockers 1, 2, 9 and 10 (Natural Advantage™, Freehold, New Jersey, U.S.A.), and SUCRAMASK™ (Creative Research Management, Stockton, California, U.S.A.). Suitable polymer additives include, but are not limited to, chitosan, pectin, pectic, pectinic, polyuronic, polygalacturonic acid, starch, food hydrocolloid or crude extracts thereof (e.g., gum acacia Senegal (FIBERGUM™), gum acacia seyal, carageenan), poly-L-lysine (e.g., poly-L-α-lysine or poly-L-ε-lysine), poly-L-ornithine (e.g., poly-L-α-ornithine or poly-L-ε-ornithine), polypropylene glycol, polyethylene glycol, poly(ethylene glycol methyl ether), polyarginine, polyaspartic acid, polyglutamic acid, polyethylene imine, alginic acid, sodium alginate, propylene glycol alginate, and sodium polyethyleneglycolalginate, sodium hexametaphosphate and its salts, and other cationic polymers and anionic polymers.

Suitable protein or protein hydrolysate additives include, but are not limited to, bovine serum albumin (BSA), whey protein (including fractions or concentrates thereof such as 90% instant whey protein isolate, 34% whey protein, 50% hydrolyzed whey protein, and 80% whey protein concentrate), soluble rice protein, soy protein, protein isolates, protein hydrolysates, reaction products of protein hydrolysates, glycoproteins, and/or proteoglycans containing amino acids (e.g., glycine, alanine, serine, threonine, asparagine, glutamine, arginine, valine, isoleucine, leucine, norvaline, methionine, proline, tyrosine, hydroxyproline, and the like), collagen (e.g., gelatin), partially hydrolyzed collagen (e.g., hydrolyzed fish collagen), and collagen hydrolysates (e.g., porcine collagen hydrolysate).

Suitable surfactant additives include, but are not limited to, polysorbates (e.g., polyoxyethylene sorbitan monooleate (polysorbate 80), polysorbate 20, polysorbate 60), sodium dodecylbenzenesulfonate, dioctyl sulfosuccinate or dioctyl sulfosuccinate sodium, sodium dodecyl sulfate, cetylpyridinium chloride (hexadecylpyridinium chloride), hexadecyltrimethylammonium bromide, sodium cholate, carbamoyl, choline chloride, sodium glycocholate, sodium taurodeoxycholate, lauric arginate, sodium stearoyl lactylate, sodium taurocholate, lecithins, sucrose oleate esters, sucrose stearate esters, sucrose palmitate esters, sucrose laurate esters, and other emulsifiers, and the like.

Suitable flavonoid additives are classified as flavonols, flavones, flavanones, flavan-3-ols, isoflavones, or anthocyanidins. Non-limiting examples of flavonoid additives include, but are not limited to, catechins (e.g., green tea extracts such as Polyphenon™ 60, Polyphenon™ 30, and Polyphenon™ 25 (Mitsui Norin Co., Ltd., Japan), polyphenols, rutins (e.g., enzyme modified rutin Sanmelin™ AO (San-fi Gen F.F.I., Inc., Osaka, Japan)), neohesperidin, naringin, neohesperidin dihydrochalcone, and the like. Suitable alcohol additives include, but are not limited to, ethanol.

Suitable astringent compound additives include, but are not limited to, tannic acid, europium chloride (EuCl₃), gadolinium chloride (GdCl₃), terbium chloride (TbCl₃), alum, tannic acid, and polyphenols (e.g., tea polyphenols).

### Sources of Selected Ingredients

In general, the ingredients such as polyphenol and the at least one essential oil(s) of the present composition can be derived from any sources either naturally or synthetically. In some embodiments, the polyphenol and/or the at least one essential oil(s) introduced into the present composition are extracted from plants and present in relatively pure form. In other embodiments, the polyphenol and/or the at least one essential oil(s) are from synthetic sources in food grade or equivalent thereof. Examples of methods for synthesizing, producing, extracting, purifying essential oil(s) and/or polyphenol are generally known in the art.

Alternatively, the polyphenol and/or the at least one essential oil(s) can be introduced into the present composition by incorporating a component that contains the desired amount of polyphenol and/or the at least one essential oil(s) into the composition. The component containing polyphenol, at least one essential oil(s), or both includes but is not limited to sage, grape, grape seed, coffee, tea, yerba mate, guayusa, yaupon, guarana, cocoa, kola, or any combination thereof.

In some embodiments, the present composition comprises a plant, or a part thereof, or a product thereof, or an extract or juice thereof, wherein the plant contains polyphenol, at least one essential oil(s), or both.

In some embodiments, the present composition comprises a first plant, or a part thereof, or a product thereof, or an extract or juice thereof, wherein the first plant contains a first polyphenol, and optionally a first essential oil(s).

In some embodiments, the present composition comprises a second plant, or a part thereof, or a product thereof, or an extract or juice thereof, wherein the second plant contains a second polyphenol, and optionally a second essential oil(s).

In some embodiments, the present composition comprises a third plant, or a part thereof, or a product thereof, or an extract or juice thereof, wherein the third plant contains a third essential oil(s).

In some embodiments, the first, second, and third plant are different from each other. In some embodiments, the first, second, and the third polyphenol are different in chemical identity. In some embodiments, the first, second, and the third polyphenol are substantially the same in chemical identity. In some embodiments, the first, second, and the third essential oil(s) compound are different in chemical identity. In some embodiments, the first, second, and the third essential oil(s) are substantially the same in chemical identity.

In some embodiments, the present composition comprises a first polyphenol and/or the at least one essential oil(s), wherein the first polyphenol and the at least one essential oil(s) are both substantially derived from sage. In some embodiments the first polyphenol and the at least one essential oil(s) are derived from different sage species. In other embodiments, at least a portion of the polyphenol and/or at least a portion of the at least one essential oil(s) is not derived from a sage source. Non-limiting examples of the sage sources include sage extract, aqueous sage extract, concentrated sage, sage juice, dried sage, soluble sage, sage oils, pulverized sage, or any combinations thereof.

In some embodiments, the present composition derived from grape seed, wherein at least the first polyphenol is substantially derived from the grape seed. In other embodiments, at least a portion of the polyphenol and/or at least a portion of the at least one essential oil(s) is not derived from grape seed. In some embodiments, the grape product is a grape seed extract, a grape seed concentrate, dried grape seed, grape seed oil, pulverized grape, or grape seed oil. In some embodiments, the grape seed source is a grape seed extract, more specifically an aqueous grape seed extract.

In some embodiments the first polyphenol and the at least one essential oil(s) are derived from the same plant species. In other embodiments the first polyphenol and the at least one essential oils(s) are derived from different plants species.

In further embodiments the at least one essential oil(s) comprises, a first essential oil, and a second essential oil, wherein the first essential oil is derived from the same or different plant species as the first polyphenol, and the second essential oil is derived from a different plant species than the first essential oil.

In some embodiments, the composition further comprises a second polyphenol, wherein the second polyphenol is not derived from the sage product. In some embodiments, the second polyphenol is derived from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof. In more specific examples embodiments, the second polyphenol is derived from tea.

In some embodiments, an edible composition comprises comprising a first polyphenol, a second polyphenol, and at least one essential oil(s), wherein the composition has a ratio of the total polyphenol to the total essential oil(s) compound of about 1:10 to about 100:1, wherein the at least one essential oil(s) comprises a first essential oil and a second essential oil. The first essential oil is derived from sage, and a second essential oils is derived from the same source or a source other than sage. Non-limiting examples of sources of essential oil can include those already described in detail above. Examples of existing known essential oils which can be included as a first or second essential oil include for example, sage oil, eucalyptus oil, rosemary oil, citrus oils, and so on.

In some embodiments, the first polyphenol comprises luteolin glycoside, luteolin glucuronide, and rosmarinic acid, wherein the total amount of luteolin glycoside is from about 0.1 mg/g to about 20 mg/g, or from about 1 mg/g to about 10 mg/g, or from about 2 mg/g to about 5 mg/g, based on the total weight of the sage product; wherein the total amount of luteolin glucuronide is from about 0.1 mg/g to about 100 mg/g, or from about 1 mg/g to about 50 mg/g, or from about 5 mg/g to about 30 mg/g, based on the total weight of the sage product; wherein the total amount of luteolin glucuronide is from about 0.1 mg/g to about 200 mg/g, or from about 1 mg/g to about 100 mg/g, or from about 5 mg/g to about 50 mg/g, based on the total weight of the sage component. In some embodiments, the sage component is a sage extract.

In some embodiments, first polyphenol is derived from sage and wherein the composition has a weight ratio of the first polyphenol derived from sage to the total essential oil(s) from about 2:1 to about 200:1. In further embodiments the first polyphenol is derived from sage and comprises at least one of luteolin glycoside, luteolin glucuronide, and rosmarinic acid.

In some embodiments, the composition of the present disclosure comprises at least one polyphenol and at least one essential oil(s), wherein the composition has a weight ratio of the total polyphenol to the total essential oil(s) from about 1:10 to about 100:1, and wherein the first polyphenol is derived from grape seed. In certain embodiments, the polyphenol derived from grape seed is obtained from grape seed extract. In embodiments the first polyphenol is derived from grape seed extract and the dosage of grape seed extract is about 50 mg to about 1,000 mg, or from about 100 mg to about 800 mg, or from about 200 mg to about 600 mg for one serving. The first polyphenol is derived from grape seed and wherein the composition has a weight ratio the first polyphenol to the total essential oil(s) from about 2:1 to about 200:1.

In some embodiments, the composition further comprises a second polyphenol, wherein the second polyphenol is not derived from the grape seed. In some embodiments, the second polyphenol is derived from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof. In some embodiments, the second polyphenol is derived from a tea product.

In some embodiments, the first polyphenol comprises luteolin glycoside, luteolin glucuronide, and rosmarinic acid, wherein the total amount of luteolin glycoside is from about 0.1 mg/g to about 20 mg/g, or from about 1 mg/g to about 10 mg/g, or from about 2 mg/g to about 5 mg/g, based on the total weight of the sage product; wherein the total amount of luteolin glucuronide is from about 0.1 mg/g to about 100 mg/g, or from about 1 mg/g to about 50 mg/g, or from about 5 mg/g to about 30 mg/g, based on the total weight of the sage product; wherein the total amount of luteolin glucuronide is from about 0.1 mg/g to about 200 mg/g, or from about 1 mg/g to about 100 mg/g, or from about 5 mg/g to about 50 mg/g, based on the total weight of the grape seed component. In some embodiments, the grape component is a grape seed extract.

In some embodiments, an edible composition comprises: a plant extract comprising a first polyphenol, a second polyphenol, and at least one essential oil(s), wherein the composition has a ratio of the total polyphenol to the total essential oil(s) of about 1:10 to about 100:1, wherein the plant extract comprises a sage extract, a grape seed extract, or both, and wherein at least a portion of the second polyphenol is not derived from the plant extract. In some embodiments, the at least a portion of the second polyphenol is derived from a plant selected from coffee, tea, yerba mate, guayusa, yaupon, guarana, cocoa, kola, berries, non-berry fruits, vegetables, seasonings, beans, nuts, soy, or any combination thereof. In some embodiments, the plant extract may be present in an amount of 50 mg to 3000 mg, 100 mg to 2000 mg, 200 mg to 1000 mg, 300 mg to 500 mg, and amounts in between these ranges. In some embodiments, the extract may be in a liquid form, a concentrate form, a powder form, a dry form, or a combination thereof.

In some embodiments, the present composition is free or substantially free from caffeine. In some embodiments, the present composition is free or substantially free from a coffee product. In some embodiments, the coffee product is selected from coffee extract, concentrated coffee, dried coffee, soluble coffee, coffee oils, coffee aromas, dry green coffee extract, wet green coffee extract, pulverized coffee, ground coffee, roast coffee, roast and ground coffee, soluble coffee, or any combinations thereof.

### Beverage

In some embodiments, the composition is a beverage selected from the group of non-carbonated beverage, carbonated beverage, juice beverage, fruit juice, coffee beverage, tea beverage, milk beverage, diary beverage, plant protein drink, plant-based beverage, sport drink, energy drink.

As used herein, a “beverage” is a ready-to-drink beverage. Suitable ready-to-drink beverages include carbonated and non-carbonated beverages. Carbonated beverages include, but are not limited to, soft drinks, cola, lemon-lime flavored sparkling beverage, orange flavored sparkling beverage, grape flavored sparkling beverage, strawberry flavored sparkling beverage, pineapple flavored sparkling beverage, ginger-ale, soft drinks, root beer and malt beverages.

Non-carbonated beverages include, but are not limited to fruit juice, fruit-flavored juice, juice drinks, nectars, vegetable juice, vegetable-flavored juice, sports drinks, energy drinks, protein drinks, enhanced water with vitamins, near water drinks (e.g., water with natural or synthetic flavorants), coconut water, tea type (e.g. black tea, green tea, red tea, oolong tea), coffee, cocoa drink, beverage containing milk components (e.g. milk beverages, coffee containing milk components, café au lait, milk tea, fruit milk beverages), beverages containing cereal extracts, smoothies and combinations thereof.

Beverages contain a liquid matrix, i.e., the basic ingredient in which the ingredients—including at least one essential oil(s) and polyphenol of the present disclosure—are dissolved. In one embodiment, the liquid matrix is water of beverage quality, such as, for example deionized water, distilled water, reverse osmosis water, carbon-treated water, purified water, demineralized water and combinations thereof, can be used. Additional suitable liquid matrices include, but are not limited to phosphoric acid, phosphate buffer, citric acid, citrate buffer and carbon-treated water.

In one embodiment, the beverage contains inclusions, i.e., pulp, seed, chunks, etc.

The beverage can further include additives including, but not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, weighing agents, juice, dairy, cereal and other plant extracts, flavonoids, alcohols, polymers and combinations thereof. Any suitable additive described herein can be used.

The beverage can further contain one or more additives and/or functional ingredients, described supra. Functional ingredients include, but are not limited to, vitamins, minerals, antioxidants, preservatives, glucosamine, and combinations thereof. Any suitable functional ingredient described herein can be used.

It is contemplated that the pH of the beverage does not materially or adversely affect the cogitation/alertness enhancement. A non-limiting example of the pH range of the beverage may be from about 1.8 to about 10. A further example includes a pH range from about 2 to about 5. In a particular embodiment, the pH of beverage can be from about 2.5 to about 4.2. One of skill in the art will understand that the pH of the beverage can vary based on the type of beverage. Dairy beverages, for example, can have pHs greater than 4.2.

The titratable acidity of the beverage may, for example, range from about 0.01 wt % to about 1.0 wt % by weight of beverage.

In one embodiment, the sparkling beverage product has an acidity from about 0.01 wt % to about 1.0 wt % by weight of the beverage, such as, for example, from about 0.05 wt % to about 0.25 wt % by weight of beverage.

The carbonation of a sparkling beverage product has 0 to about 2% (w/w) of carbon dioxide or its equivalent, for example, from about 0.1 to about 1.0% (w/w).

The temperature of the beverage may, for example, range from about 4 °C to about 100 °C, such as, for example, from about 4 °C to about 25 °C.

The beverage can be a full-calorie beverage that has up to about 120 calories per 8 oz serving. The beverage can be a mid-calorie beverage that has up to about 60 calories per 8 oz serving. The beverage can be a low-calorie beverage that has up to about 40 calories per 8 oz serving. The beverage can be a zero-calorie that has less than about 5 calories per 8 oz. serving.

In some embodiments, the present disclosure relates to a beverage comprising a first polyphenol from about 0.002 wt % to about 2 wt %; and from about 0.001 wt % to about 1 wt % of at least one essential oil(s), wherein the beverage has a weight ratio of total polyphenol to the total essential oil(s) from about 1:10 to about 100:1. In some embodiments, the composition further comprises from about 0.002 wt % to about 2 wt % of a second polyphenol, wherein the first and the second polyphenol are not derived from the same plant extract. In some embodiments, the second polyphenol is not derived from the plant extract comprising the first polyphenol. In some embodiments, the second polyphenol is not derived from a sage source or a grape seed source.

### Edible Gel Mixes and Edible Gel Compositions

In one embodiment, the president composition is an edible gel or edible gel mix. Edible gels are gels that can be eaten. A gel is a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels can be used in a number of applications. For example, gels can be used in foods, paints, and adhesives.

Non-limiting examples of edible gel compositions for use in particular embodiments include gel desserts, puddings, jellies, pastes, trifles, aspics, marshmallows, gummy candies, or the like. Edible gel mixes generally are powdered or granular solids to which a fluid may be added to form an edible gel composition. Non-limiting examples of fluids for use in particular embodiments include water, dairy fluids, dairy analogue fluids, juices, alcohol, alcoholic beverages, and combinations thereof. Non-limiting examples of dairy fluids which may be used in particular embodiments include milk, cultured milk, cream, fluid whey, and mixtures thereof. Non-limiting examples of dairy analogue fluids which may be used in particular embodiments include, for example, soy milk and non-dairy coffee whitener. Because edible gel products found in the marketplace typically are sweetened with sucrose, it is desirable to sweeten edible gels with an alternative sweetener in order provide a low-calorie or non-calorie alternative.

As used herein, the term “gelling ingredient” denotes any material that can form a colloidal system within a liquid medium. Non-limiting examples of gelling ingredients for use in particular embodiments include gelatin, alginate, carageenan, gum, pectin, konjac, agar, food acid, rennet, starch, starch derivatives, and combinations thereof. It is well known to those having ordinary skill in the art that the amount of gelling ingredient used in an edible gel mix or an edible gel composition varies considerably depending on a number of factors, such as the particular gelling ingredient used, the particular fluid base used, and the desired properties of the gel.

Non-limiting examples of other ingredients for use in particular embodiments include a food acid, a salt of a food acid, a buffering system, a bulking agent, a sequestrant, a cross-linking agent, one or more flavors, one or more colors, and combinations thereof. Non-limiting examples of food acids for use in particular embodiments include citric acid, adipic acid, fumaric acid, lactic acid, malic acid, and combinations thereof. Non-limiting examples of salts of food acids for use in particular embodiments include sodium salts of food acids, potassium salts of food acids, and combinations thereof. Non-limiting examples of bulking agents for use in particular embodiments include raftilose, isomalt, sorbitol, polydextrose, maltodextrin, and combinations thereof. Non-limiting examples of sequestrants for use in particular embodiments include calcium disodium ethylene tetra-acetate, glucono delta-lactone, sodium gluconate, potassium gluconate, ethylenediaminetetraacetic acid (EDTA), and combinations thereof. Non-limiting examples of cross-linking agents for use in particular embodiments include calcium ions, magnesium ions, sodium ions, and combinations thereof.

### Confections

In one embodiment, the present composition is a confection.

As referred to herein, “confection” can mean a sweet, a lollie, a confectionery, or similar term. The confection generally contains a base composition component and a sweetener component. The confection may be in the form of any food that is typically perceived to be rich in sugar or is typically sweet. According to particular embodiments of the present disclosure, the confections may be bakery products such as pastries; desserts such as yogurt, jellies, drinkable jellies, puddings, Bavarian cream, blancmange, cakes, brownies, mousse and the like, sweetened food products eaten at tea time or following meals; frozen foods; cold confections, e.g. types of ice cream such as ice cream, ice milk, lacto-ice and the like (food products in which sweeteners and various other types of raw materials are added to milk products, and the resulting mixture is agitated and frozen), and ice confections such as sherbets, dessert ices and the like (food products in which various other types of raw materials are added to a sugary liquid, and the resulting mixture is agitated and frozen); general confections, e.g., baked confections or steamed confections such as crackers, biscuits, buns with bean-jam filling, halvah, alfajor, and the like; rice cakes and snacks; table top products; general sugar confections such as chewing gum (e.g. including compositions which comprise a substantially water-insoluble, chewable gum base, such as chicle or substitutes thereof, including jetulong, guttakay rubber or certain comestible natural synthetic resins or waxes), hard candy, soft candy, mints, nougat candy, jelly beans, fudge, toffee, taffy, Swiss milk tablet, licorice candy, chocolates, gelatin candies, marshmallow, marzipan, divinity, cotton candy, and the like; sauces including fruit flavored sauces, chocolate sauces and the like; edible gels; crémes including butter crémes, flour pastes, whipped cream and the like; jams including strawberry jam, marmalade and the like; and breads including sweet breads and the like or other starch products, and combinations thereof.

As referred to herein, “base composition” means any composition which can be a food item and provides a matrix for carrying the sweetener component.

Suitable base compositions for embodiments of this disclosure may include flour, yeast, water, salt, butter, eggs, milk, milk powder, liquor, gelatin, nuts, chocolate, citric acid, tartaric acid, fumaric acid, natural flavors, artificial flavors, colorings, polyols, sorbitol, isomalt, maltitol, lactitol, malic acid, magnesium stearate, lecithin, hydrogenated glucose syrup, glycerine, natural or synthetic gum, starch, and the like, and combinations thereof. Such components generally are recognized as safe (GRAS) and/or are U.S. Food and Drug Administration (FDA)-approved. According to particular embodiments of the invention, the base composition is present in the confection in an amount ranging from about 0.1 to about 99 weight percent of the confection.

The base composition of the confection may optionally include other artificial or natural sweeteners, bulk sweeteners, or combinations thereof. Bulk sweeteners include both caloric and non-caloric compounds. Non-limiting examples of bulk sweeteners include sucrose, dextrose, maltose, dextrin, dried invert sugar, fructose, high fructose corn syrup, levulose, galactose, corn syrup solids, tagatose, polyols (e.g., sorbitol, mannitol, xylitol, lactitol, erythritol, and maltitol), hydrogenated starch hydrolysates, isomalt, trehalose, and mixtures thereof. Generally, the amount of bulk sweetener present in the confection ranges widely depending on the particular embodiment of the confection and the desired degree of sweetness. Those of ordinary skill in the art will readily ascertain the appropriate amount of bulk sweetener.

### Condiment Compositions

In one embodiment, the present composition is a condiment. In another embodiment, a condiment comprises a composition of the present disclosure. Condiments, as used herein, are compositions used to enhance or improve the flavor of a food or beverage. Non-limiting examples of condiments include ketchup (catsup); mustard; barbecue sauce; butter; chili sauce; chutney; cocktail sauce; curry; dips; fish sauce; horseradish; hot sauce; jellies, jams, marmalades, or preserves; mayonnaise; peanut butter; relish; remoulade; salad dressings (e.g., oil and vinegar, Caesar, French, ranch, bleu cheese, Russian, Thousand Island, Italian, and balsamic vinaigrette), salsa; sauerkraut; soy sauce; steak sauce; syrups; tartar sauce; and Worcestershire sauce.

Condiment bases generally comprise a mixture of different ingredients, non-limiting examples of which include vehicles (e.g., water and vinegar); spices or seasonings (e.g., salt, pepper, garlic, mustard seed, onion, paprika, turmeric, and combinations thereof); fruits, vegetables, or their products (e.g., tomatoes or tomato-based products (paste, puree), fruit juices, fruit juice peels, and combinations thereof);

oils or oil emulsions, particularly vegetable oils; thickeners (e.g., xanthan gum, food starch, other hydrocolloids, and combinations thereof); and emulsifying agents (e.g., egg yolk solids, protein, gum arabic, carob bean gum, guar gum, gum karaya, gum tragacanth, carageenan, pectin, propylene glycol esters of alginic acid, sodium carboxymethyl-cellulose, polysorbates, and combinations thereof). Recipes for condiment bases and methods of making condiment bases are well known to those of ordinary skill in the art.

Generally, condiments also comprise caloric sweeteners, such as sucrose, high fructose corn syrup, molasses, honey, or brown sugar. In exemplary embodiments of the condiments provided herein, a compound of the present invention or a composition comprising the same is used instead of traditional caloric sweeteners. Accordingly, a condiment composition desirably comprises a compound of the present invention or a composition comprising a compound of the present invention and a condiment base.

The condiment composition optionally may include other natural and/or synthetic high-potency sweeteners, bulk sweeteners, pH modifying agents (e.g., lactic acid, citric acid, phosphoric acid, hydrochloric acid, acetic acid, and combinations thereof), fillers, functional agents (e.g., pharmaceutical agents, nutrients, or components of a food or plant), flavorings, colorings, or combinations thereof.

### Chewing Gum Compositions

In one embodiment, the present composition is a chewing gum. In another embodiment, a chewing gum composition comprises a composition of the present disclosure. Chewing gum compositions generally comprise a water-soluble portion and a water-insoluble chewable gum base portion. The water-soluble portion, which typically includes the composition of the present disclosure, dissipates with a portion of the flavoring agent over a period of time during chewing while the insoluble gum base portion is retained in the mouth. The insoluble gum base generally determines whether a gum is considered chewing gum, bubble gum, or a functional gum.

The insoluble gum base, which is generally present in the chewing gum composition in an amount in the range of about 15 to about 35 weight percent of the chewing gum composition, generally comprises combinations of elastomers, softeners (plasticizers), emulsifiers, resins, and fillers. Such components generally are considered food grade, recognized as safe (GRA), and/or are U.S. Food and Drug Administration (FDA)-approved.

Elastomers, the primary component of the gum base, provide the rubbery, cohesive nature to gums and can include one or more natural rubbers (e.g., smoked latex, liquid latex, or guayule); natural gums (e.g., jelutong, perillo, sorva, massaranduba balata, massaranduba chocolate, nispero, rosindinha, chicle, and gutta hang kang); or synthetic elastomers (e.g., butadiene-styrene copolymers, isobutylene-isoprene copolymers, polybutadiene, polyisobutylene, and vinyl polymeric elastomers). In a particular embodiment, the elastomer is present in the gum base in an amount in the range of about 3 to about 50 weight percent of the gum base.

Resins are used to vary the firmness of the gum base and aid in softening the elastomer component of the gum base. Non-limiting examples of suitable resins include a rosin ester, a terpene resin (e.g., a terpene resin from α-pinene, β-pinene and/or d-limonene), polyvinyl acetate, polyvinyl alcohol, ethylene vinyl acetate, and vinyl acetate-vinyl laurate copolymers. Non-limiting examples of rosin esters include a glycerol ester of a partially hydrogenated rosin, a glycerol ester of a polymerized rosin, a glycerol ester of a partially dimerized rosin, a glycerol ester of rosin, a pentaerythritol ester of a partially hydrogenated rosin, a methyl ester of rosin, or a methyl ester of a partially hydrogenated rosin. In a particular embodiment, the resin is present in the gum base in an amount in the range of about 5 to about 75 weight percent of the gum base. Softeners, which also are known as plasticizers, are used to modify the ease of chewing and/or mouth feel of the chewing gum composition. Generally, softeners comprise oils, fats, waxes, and emulsifiers. Non-limiting examples of oils and fats include tallow, hydrogenated tallow, large, hydrogenated or partially hydrogenated vegetable oils (e.g., soybean, canola, cottonseed, sunflower, palm, coconut, corn, safflower, or palm kernel oils), cocoa butter, glycerol monostearate, glycerol triacetate, glycerol abietate, leithin, monoglycerides, diglycerides, triglycerides acetylated monoglycerides, and free fatty acids. Non-limiting examples of waxes include polypropylene/polyethylene/Fisher-Tropsch waxes, paraffin, and microcrystalline and natural waxes (e.g., candelilla, beeswax and carnauba). Microcrystalline waxes, especially those with a high degree of crystallinity and a high melting point, also may be considered as bodying agents or textural modifiers. In a particular embodiment, the softeners are present in the gum base in an amount in the range of about 0.5 to about 25 weight percent of the gum base.

Emulsifiers are used to form a uniform dispersion of the insoluble and soluble phases of the chewing gum composition and also have plasticizing properties. Suitable emulsifiers include glycerol monostearate (GMS), lecithin (Phosphatidyl choline), polyglycerol polyricinoleic acid (PPGR), mono and diglycerides of fatty acids, glycerol distearate, tracetin, acetylated monoglyceride, glycerol triactetate, and magnesium stearate. In a particular embodiment, the emulsifiers are present in the gum base in an amount in the range of about 2 to about 30 weight percent of the gum base.

The chewing gum composition also may comprise adjuvants or fillers in either the gum base and/or the soluble portion of the chewing gum composition. Suitable adjuvants and fillers include lecithin, inulin, polydextrin, calcium carbonate, magnesium carbonate, magnesium silicate, ground limestome, aluminum hydroxide, aluminum silicate, talc, clay, alumina, titanium dioxide, and calcium phosphate. In particular embodiments, lecithin can be used as an inert filler to decrease the stickiness of the chewing gum composition. In other particular embodiments, lactic acid copolymers, proteins (e.g., gluten and/or zein) and/or guar can be used to create a gum that is more readily biodegradable. The adjuvants or fillers are generally present in the gum base in an amount up to about 20 weight percent of the gum base. Other optional ingredients include coloring agents, whiteners, preservatives, and flavors.

In particular embodiments of the chewing gum composition, the gum base comprises about 5 to about 95 weight percent of the chewing gum composition, more desirably about 15 to about 50 weight percent of the chewing gum composition, and even more desirably from about 20 to about 30 weight percent of the chewing gum composition.

The soluble portion of the chewing gum composition may optionally include other artificial or natural sweeteners, bulk sweeteners, softeners, emulsifiers, flavoring agents, coloring agents, adjuvants, fillers, functional agents (e.g., pharmaceutical agents or nutrients), or combinations thereof. Suitable examples of softeners and emulsifiers are described above.

Bulk sweeteners include both caloric and non-caloric compounds. Non-limiting examples of bulk sweeteners include sucrose, dextrose, maltose, dextrin, dried invert sugar, fructose, high fructose corn syrup, levulose, galactose, corn syrup solids, tagatose, polyols (e.g., sorbitol, mannitol, xylitol, lactitol, erythritol, and maltitol), hydrogenated starch hydrolysates, isomalt, trehalose, and mixtures thereof. In particular embodiments, the bulk sweetener is present in the chewing gum composition in an amount in the range of about 1 to about 75 weight percent of the chewing gum composition.

Flavoring agents may be used in either the insoluble gum base or soluble portion of the chewing gum composition. Such flavoring agents may be natural or artificial flavors. In a particular embodiment, the flavoring agent comprises an essential oil, such as an oil derived from a plant or a fruit, peppermint oil, spearmint oil, other mint oils, clove oil, cinnamon oil, oil of wintergreen, bay, thyme, cedar leaf, nutmeg, allspice, sage, mace, and almonds. In another particular embodiment, the flavoring agent comprises a plant extract or a fruit essence such as apple, banana, watermelon, pear, peach, grape, strawberry, raspberry, cherry, plum, pineapple, apricot, and mixtures thereof. In still another particular embodiment, the flavoring agent comprises a citrus flavor, such as an extract, essence, or oil of lemon, lime, orange, tangerine, grapefruit, citron, or kumquat.

### Cereal Compositions

In one embodiment, the present composition is a cereal composition. In another embodiment, the present disclosure relates to a cereal composition that comprises a composition comprising polyphenol and at least one essential oil(s). Cereal compositions typically are eaten either as staple foods or as snacks. Non-limiting examples of cereal compositions for use in particular embodiments include ready-to-eat cereals as well as hot cereals. Ready-to-eat cereals are cereals which may be eaten without further processing (i.e. cooking) by the consumer. Examples of ready-to-eat cereals include breakfast cereals and snack bars. Breakfast cereals typically are processed to produce a shredded, flaky, puffy, or extruded form. Breakfast cereals generally are eaten cold and are often mixed with milk and/or fruit. Snack bars include, for example, energy bars, rice cakes, granola bars, and nutritional bars. Hot cereals generally are cooked, usually in either milk or water, before being eaten. Non-limiting examples of hot cereals include grits, porridge, polenta, rice, and rolled oats.

Cereal compositions generally comprise at least one cereal ingredient. As used herein, the term “cereal ingredient” denotes materials such as whole or part grains, whole or part seeds, and whole or part grass. Non-limiting examples of cereal ingredients for use in particular embodiments include maize, wheat, rice, barley, bran, bran endosperm, bulgur, soghums, millets, oats, rye, triticale, buckwheat, fonio, quinoa, bean, soybean, amaranth, teff, spelt, and kaniwa.

In a particular embodiment, the cereal composition comprises polyphenol and at least one essential oil(s) according to the present disclosure or a composition comprising at least one essential oil(s) and polyphenol and at least one cereal ingredient. The at least one essential oil(s) polyphenol or the composition comprising at least one essential oil(s) and polyphenol may be added to the cereal composition in a variety of ways, such as, for example, as a coating, as a frosting, as a glaze, or as a matrix blend (i.e. added as an ingredient to the cereal formulation prior to the preparation of the final cereal product).

Accordingly, in a particular embodiment, at least one essential oil(s) and polyphenol or a composition comprising at least one essential oil(s) and polyphenol is added to the cereal composition as a matrix blend. In one embodiment, the at least one essential oil(s) and polyphenol or a composition comprising essential oil compound and polyphenol are blended with a cereal prior to cooking to provide a cereal product. In another embodiment, at least one essential oil(s) and polyphenol or a composition comprising at least one essential oil(s) and polyphenol are blended with the cereal matrix before the cereal is extruded.

In another particular embodiment, at least one essential oil(s) and polyphenol or a composition comprising at least one essential oil(s) and polyphenol are added to the cereal composition as a coating, such as, for example, by combining at least one essential oil(s) and polyphenol or a comprising at least one essential oil(s) and polyphenol with a food grade oil and applying the mixture onto the cereal. In a different embodiment, at least one essential oil(s) and polyphenol or a composition comprising at least one essential oil(s) and polyphenol and the food grade oil may be applied to the cereal separately, by applying either the oil or the sweetener first. Non-limiting examples of food grade oils for use in particular embodiments include vegetable oils such as corn oil, soybean oil, cottonseed oil, peanut oil, coconut oil, canola oil, olive oil, sesame seed oil, palm oil, palm kernel oil, and mixtures thereof. In yet another embodiment, food grade fats may be used in place of the oils, provided that the fat is melted prior to applying the fat onto the cereal.

In another embodiment, at least one essential oil(s) and polyphenol or a composition comprising at least one essential oil(s) and polyphenol are added to the cereal composition as a glaze. Non-limiting examples of glazing agents for use in particular embodiments include corn syrup, honey syrups and honey syrup solids, maple syrups and maple syrup solids, sucrose, isomalt, polydextrose, polyols, hydrogenated starch hydrolysate, aqueous solutions thereof, and mixtures thereof. In another such embodiment, at least one essential oil(s) and polyphenol or a composition comprising at least one essential oil(s) and polyphenol are added as a glaze by combining with a glazing agent and a food grade oil or fat and applying the mixture to the cereal. In yet another embodiment, a gum system, such as, for example, gum acacia, carboxymethyl cellulose, or algin, may be added to the glaze to provide structural support. In addition, the glaze also may include a coloring agent, and also may include a flavor.

In another embodiment, at least one essential oil(s) and polyphenol or a composition comprising at least one essential oil(s) and polyphenol is added to the cereal composition as a frosting. In one such embodiment, at least one essential oil(s) and polyphenol or a composition comprising at least one essential oil(s) and polyphenol is combined with water and a frosting agent and then applied to the cereal. Non-limiting examples of frosting agents for use in particular embodiments include maltodextrin, sucrose, starch, polyols, and mixtures thereof. The frosting also may include a food grade oil, a food grade fat, a coloring agent, and/or a flavor.

### Dairy Products

In one embodiment, the composition of the present disclosure is a dairy product that comprises at least one essential oil(s) and polyphenol described herein. In another embodiment, the composition of the present disclosure is a dairy product that comprises a composition comprising at least one essential oil(s) and polyphenol. Dairy products and processes for making dairy products suitable for use in this invention are well known to those of ordinary skill in the art. Dairy products, as used herein, comprise milk or foodstuffs produced from milk. Non-limiting examples of dairy products suitable for use in embodiments of this invention include milk, milk cream, sour cream, creme fraiche, buttermilk, cultured buttermilk, milk powder, condensed milk, evaporated milk, butter, cheese, cottage cheese, cream cheese, yogurt, ice cream, frozen custard, frozen yogurt, gelato, via, piima, filmjlk, kajmak, kephir, viili, kumiss, airag, ice milk, casein, ayran, lassi, khoa, or combinations thereof.

Milk is a fluid secreted by the mammary glands of female mammals for the nourishment of their young. The female ability to produce milk is one of the defining characteristics of mammals and provides the primary source of nutrition for newborns before they are able to digest more diverse foods. In particular embodiments of this invention, the dairy products are derived from the raw milk of cows, goats, sheep, horses, donkeys, camels, water buffalo, yaks, reindeer, moose, or humans.

In particular embodiments of this invention, the processing of the dairy product from raw milk generally comprises the steps of pasteurizing, creaming, and homogenizing. Although raw milk may be consumed without pasteurization, it usually is pasteurized to destroy harmful microorganisms such as bacteria, viruses, protozoa, molds, and yeasts. Pasteurizing generally comprises heating the milk to a high temperature for a short period of time to substantially reduce the number of microorganisms, thereby reducing the risk of disease.

Creaming traditionally follows pasteurization step and involves the separation of milk into a higher-fat cream layer and a lower-fat milk layer. Milk will separate into milk and cream layers upon standing for twelve to twenty-four hours. The cream rises to the top of the milk layer and may be skimmed and used as a separate dairy product. Alternatively, centrifuges may be used to separate the cream from the milk. The remaining milk is classified according to the fat content of the milk, non-limiting examples of which include whole, 2%, 1%, and skim milk.

After removing the desired amount of fat from the milk by creaming, milk is often homogenized. Homogenization prevents cream from separating from the milk and generally involves pumping the milk at high pressures through narrow tubes in order to break up fat globules in the milk. Pasteurization, creaming, and homogenization of milk are common but are not required to produce consumable dairy products. Accordingly, suitable dairy products for use in embodiments of this invention may undergo no processing steps, a single processing step, or combinations of the processing steps described herein. Suitable dairy products for use in embodiments of this invention may also undergo processing steps in addition to or apart from the processing steps described herein.

Particular embodiments of this invention comprise dairy products produced from milk by additional processing steps. As described above, cream may be skimmed from the top of milk or separated from the milk using machine-centrifuges. In a particular embodiment, the dairy product comprises sour cream, a dairy product rich in fats that is obtained by fermenting cream using a bacterial culture. The bacteria produce lactic acid during fermentation, which sours and thickens the cream. In another particular embodiment, the dairy product comprises creme fraiche, a heavy cream slightly soured with bacterial culture in a similar manner to sour cream. Crème fraiche ordinarily is not as thick or as sour as sour cream. In yet another particular embodiment, the dairy product comprises cultured buttermilk. Cultured buttermilk is obtained by adding bacteria to milk. The resulting fermentation, in which the bacterial culture turns lactose into lactic acid, gives cultured buttermilk a sour taste. Although it is produced in a different manner, cultured buttermilk generally is similar to traditional buttermilk, which is a by-product of butter manufacture.

According to other particular embodiments of this invention, the dairy products comprise milk powder, condensed milk, evaporated milk, or combinations thereof. Milk powder, condensed milk, and evaporated milk generally are produced by removing water from milk. In a particular embodiment, the dairy product comprises a milk powder comprising dried milk solids with a low moisture content. In another particular embodiment, the dairy product comprises condensed milk. Condensed milk generally comprises milk with a reduced water content and added sweetener, yielding a thick, sweet product with a long shelf-life. In yet another particular embodiment, the dairy product comprises evaporated milk. Evaporated milk generally comprises fresh, homogenized milk from which about 60% of the water has been removed, that has been chilled, fortified with additives such as vitamins and stabilizers, packaged, and finally sterilized. According to another particular embodiment of this invention, the dairy product comprises a dry creamer and at least one essential oil(s) and polyphenol or a composition comprising at least one essential oil(s) and polyphenol.

In another particular embodiment, the dairy product provided herein comprises butter. Butter generally is made by churning fresh or fermented cream or milk. Butter generally comprises butterfat surrounding small droplets comprising mostly water and milk proteins. The churning process damages the membranes surrounding the microscopic globules of butterfat, allowing the milk fats to conjoin and to separate from the other parts of the cream. In yet another particular embodiment, the dairy product comprises buttermilk, which is the sour-tasting liquid remaining after producing butter from full-cream milk by the churning process.

In still another particular embodiment, the dairy product comprises cheese, a solid foodstuff produced by curdling milk using a combination of rennet or rennet substitutes and acidification. Rennet, a natural complex of enzymes produced in mammalian stomachs to digest milk, is used in cheese-making to curdle the milk, causing it to separate into solids known as curds and liquids known as whey. Generally, rennet is obtained from the stomachs of young ruminants, such as calves; however, alternative sources of rennet include some plants, microbial organisms, and genetically modified bacteria, fungus, or yeast. In addition, milk may be coagulated by adding acid, such as citric acid. Generally, a combination of rennet and/or acidification is used to curdle the milk. After separating the milk into curds and whey, some cheeses are made by simply draining, salting, and packaging the curds. For most cheeses, however, more processing is needed. Many different methods may be used to produce the hundreds of available varieties of cheese. Processing methods include heating the cheese, cutting it into small cubes to drain, salting, stretching, cheddaring, washing, molding, aging, and ripening. Some cheeses, such as the blue cheeses, have additional bacteria or molds introduced to them before or during aging, imparting flavor and aroma to the finished product. Cottage cheese is a cheese curd product with a mild flavor that is drained but not pressed so that some whey remains. The curd is usually washed to remove acidity. Cream cheese is a soft, mild-tasting, white cheese with a high fat content that is produced by adding cream to milk and then curdling to form a rich curd. Alternatively, cream cheese can be made from skim milk with cream added to the curd. It should be understood that cheese, as used herein, comprises all solid foodstuff produced by the curdling milk.

In another particular embodiment of this invention, the dairy product comprises yogurt. Yogurt generally is produced by the bacterial fermentation of milk. The fermentation of lactose produces lactic acid, which acts on proteins in milk to give the yogurt a gel-like texture and tartness. In particularly desirable embodiments, the yogurt may be sweetened with a sweetener and/or flavored. Non-limiting examples of flavorings include, but are not limited to, fruits (e.g., peach, strawberry, banana), vanilla, and chocolate. Yogurt, as used herein, also includes yogurt varieties with different consistencies and viscosities, such as dahi, dadih or dadiah, labneh or labaneh, bulgarian, kefir, and matsoni. In another particular embodiment, the dairy product comprises a yogurt-based beverage, also known as drinkable yogurt or a yogurt smoothie. In particularly desirable embodiments, the yogurt-based beverage may comprise sweeteners, flavorings, other ingredients, or combinations thereof.

Other dairy products beyond those described herein may be used in particular embodiments of this invention. Such dairy products are well known to those of ordinary skill in the art, non-limiting examples of which include milk, milk and juice, coffee, tea, via, piima, filmjolk, kajmak, kephir, viili, kumiss, airag, ice milk, casein, ayran, lassi, and khoa.

According to particular embodiments of this invention, the dairy compositions also may comprise other additives. Non-limiting examples of suitable additives include sweeteners and flavorants such as chocolate, strawberry, and banana. Particular embodiments of the dairy compositions provided herein also may comprise additional nutritional supplements such as vitamins (e.g., vitamin D) and minerals (e.g., calcium) to improve the nutritional composition of the milk.

In some embodiment, the present composition is a non-diary milk, a plant-based drink, a plant protein drink, a plant milk such as soy milk, almond milk, rice milk, soybean milk, coconut milk, workpiece plant milk, and the like.

### Tabletop Functional Sweetener Compositions

In some embodiments, the present composition is a tabletop functional sweetener composition comprising at least one essential oil(s) and polyphenol in combination with: (i) at least one functional ingredient; (ii) at least one bulking agent; and (iii) optionally at least one sweet taste improving composition and/or anti-caking agent with improved temporal and/or flavor profile. In accordance with particular embodiments, suitable “bulking agents” include maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, and mixtures thereof. Additionally, in accordance with still other embodiments of the invention, granulated sugar (sucrose) or other caloric sweeteners such as crystalline fructose, other carbohydrates, or sugar alcohols can be used as a bulking agent due to their provision of good content uniformity without the addition of significant calories.

In one embodiment, a bulking agent may be used as a sweet taste improving composition.

As used herein the phrase “anti-caking agent” and “flow agent” refer to any composition which prevents, reduces, inhibits, or suppresses at least one natural and/or synthetic high-potency sweetener molecule from attaching, binding, or contacting to another natural and/or synthetic high-potency sweetener molecule. Alternatively, anti-caking agent may refer to any composition which assists in content uniformity and uniform dissolution. In accordance with particular embodiments, non-limiting examples of anti-caking agents include cream of tartar, calcium silicate, silicon dioxide, microcrystalline cellulose (Avicel, FMC BioPolymer, Philadelphia, Pa.), and tricalcium phosphate. In one embodiment, the anti-caking agents are present in the tabletop functional sweetener composition in an amount from about 0.001 to about 3% by weight of the tabletop functional sweetener composition.

Tabletop functional sweetener compositions are embodied and packaged in numerous different forms and it is intended that the tabletop functional sweetener compositions of the present invention may be of any form known in the art. In accordance with particular embodiments, non-limiting examples include powder form, granular form, packets, tablets, sachets, pellets, cubes, solids, and liquids. In an embodiment, a tabletop functional sweetener composition comprises a single-serving (portion control) packet comprising a dry-blend of a functional sweetener formulation. Dry-blend formulations generally may comprise powder or granules. Although the tabletop functional sweetener packet may be of any size, an illustrative non-limiting example of conventional portion control tabletop sweetener packets are approximately 2.5 by 1.5 inches and hold approximately 1 gram of a sweetener composition having a sweetness equivalent to 2 teaspoons of granulated sugar (about 8 g). The amount of natural and/or synthetic high-potency sweetener in a dry-blend tabletop functional sweetener formulation will vary due to the varying potency of different natural and/or synthetic high-potency sweeteners. In a particular embodiment, a dry-blend tabletop functional sweetener formulation may comprise a natural and/or synthetic high-potency sweetener in an amount from about 1% (w/w) to about 10% (w/w) of the tabletop functional sweetener composition.

Solid tabletop functional sweetener embodiments include cubes and tablets. A non-limiting example of conventional cubes are equivalent in size to a standard cube of granulated sugar, which is approximately 2.2×2.2×2.2 cm³ and weigh approximately 8 g. In one embodiment, a solid tabletop sweetener is in the form of a tablet or any other four known to those skilled in the art.

In one embodiment, the tabletop functional sweetener composition may also be formulated for targeted uses, for example, in beverage, food, pharmaceutical, cosmetics, herbal/vitamins, tobacco, and in any other products which may be sweetened. For example, a tabletop functional sweetener composition for baking may be formulated having additional protecting agents such as encapsulants. Other forms will be readily apparent to those skilled in the tabletop sweetener art.

Commonly used methods for making powder or granulated functional sweetener formulations for packets include fluid bed agglomeration processes. Other methods for making tabletop sweetener compositions are well known to those of ordinary skill in the art.

### Methods

In another aspect, the present disclosure relates to a method for improving a condition of a human, the method comprising: administering to the human in need of an improved condition an edible composition described anywhere herein. In one particular embodiment, the composition comprises: a first polyphenol; and at least one essential oil(s), wherein a ratio of the total polyphenol to the total essential oil(s) is from about 1:10 to about 100:1. In some embodiments, the composition is free of substantially free from caffeine.

In some embodiments, the methods described herein cause one or more effects on the human, such as: improving cognitive function; improving mood; improving happiness; improving friendliness; improving sociability; improving relaxation, increasing euphoria; improving conviviality; improving alertness; improving contentedness; improving calmness; improving tranquility; improving mental capability; improving memory accuracy; improving speed of memory; improving relaxation; improving/sustaining attention; improving accuracy of attention; improving stroop effect, improving speed of attention; reducing stress; reducing tension; reducing mental fatigue; reducing anxiety; reducing inertia; reducing headache; or any combinations thereof.

In some embodiments a method is disclosed for improving the condition of a human subject the method comprising: administering to the human in need of an improved condition a beverage comprising: a first polyphenol; and at least one essential oil(s), wherein the beverage has a weight ratio of the total polyphenol to total essential oil(s) from about 1:10 to about 100:1, and wherein the first polyphenol is derived from sage.

In other embodiments a method is disclosed for improving the condition of a human subject the method comprising: administering to the human in need of an improved condition a beverage comprising: a first polyphenol; and at least one essential oil(s), wherein the beverage has a weight ratio of the total polyphenol to total essential oil(s) from about 1:10 to about 100:1, and wherein the first polyphenol is derived from grape seed.

In some embodiments, the composition is administered orally by having the human ingest the composition. The composition may be administered at one time, or alternatively more than one time a day. The administration can continue for at least 1 day, at least 1 week, at least 1 month, at least 1 year. In some embodiments, the methods comprise applying a time interval between any two administrations per day, wherein the time interval is at least about 1 hour, at least about 2 hours, at least about 3 hours, at least about 4 hours, at least about 5 hours, or at least about 6 hours.

In some embodiments of the described methods herein, the composition or beverage takes effect after a time period following the administration, wherein the time period is about 5 minutes, or about 15 minutes, or about 30 minutes, or about 60 minutes, or about 90 minutes, or about 120 minutes or about 150 minutes, or about 200 minutes, or about 250 minutes, or about 300 minutes

In some embodiments, the composition administered to the human has a dosage of at least one essential oil(s) of at least about 3.0 mg, at least about 50 mg, at least about 100 mg, at least about 150 mg or any value therebetween. In some embodiments, the composition administered to the human has a dosage of polyphenol of at least about 15 mg, at least about 50 mg, at least about 100 mg, at least about 200 mg, at least about 250 mg, at least about 300 mg, or any value therebetween.

### NON-PATENT REFERENCES

Jackson, P. A., Haskell-Ramsay, C., Forster, J., Khan, J., Veasey, R., Kennedy, D. O., Wilson, A. R., Saunders, C., Wightman, E. L., Acute cognitive performance and mood effects of coffee berry and apple extracts: A randomised, double blind, placebo-controlled crossover study
   in healthy humans, Nutritional Neuroscience, 2021, DOI: 10.1080/1028415X.2021.1963068.
Ward-Ritacco, C. L., Wilson, A. R., O'Connor, P. J., An Apple Extract Beverage Combined with Caffeine Can Improve Alertness, Mental Fatigue, and Information Processing Speed. Journal of Cognitive Enhancement, 2021, https://doi.org/10.1007/s41465-020-00204-1.
Reed. R. A., Mitchell, E. S., Saunders, C., and O'Connor, P., Acute Low and Moderate Doses of a Caffeine-Free Polyphenol-Rich Coffeeberry Extract Improve Feelings of Alertness and Fatigue Resulting from the Performance of Fatiguing Cognitive Tasks. Journal of Cognitive Enhancement, 2019, 3, 193-206.
McGranahan, M., Nuccio, R., Mitchell, E., Pahnke, M., and O'Connor, P., Acute Effects of Two Doses of a Coffeeberry Extract on Mental Energy-Related Feelings and Cycling Performance After Cognitive and Physical Work. Current Developments in Nutrition, 2021, 5 (Supplemental 2), 1294.

All publications, patents and patent applications mentioned in the specification are indicative of the level of those skilled in the art to which this disclosure pertains.

The following examples illustrate preferred, but non-limiting embodiments of the present disclosure.

### EXAMPLES

### Example 1—Study on Acute Effects of Compositions Containing on Cognitive Function and Alertness in Healthy Adults

### Background and Objective

Polyphenols and some essential oil compounds are reportedly beneficial to human. However, their acute effects in cognitive function improvement are not noticeable based on prior disclosure.

The aim of the proposed randomized, double-blind, placebo-controlled, balanced cross-over methodology is to assess the acute effects of composition systems having both polyphenol and selected essential oil compounds on cognitive and mood function. The trial utilized the Cognimapp, an online cognitive assessment system. The main cognitive/mood assessments will take place pre-dose and at 60 min post-dose on four separate testing days separated by a minimum of 1-day washout.

### Study Population

Three studies were conducted with total participants of 9 for study 1, total participants of 36 for study 2, and total participants of 30 for study 3. Respondents were recruited by web survey using the inclusion and exclusion criteria.

The following criteria are used to selected participants for the study:
Participants are volunteering for the trials;
Participants must self-assess themselves as being in good health;
Aged 21 to 60 years at the time of giving consent;
Participants agree to stay engaged in the tasks across all tests, which can be identified from the dataset.

Participants are not eligible to take part if they:
Have any pre-existing medical condition/illness (e.g., feel sick, have reduced brain performance, such as focus, memory, accuracy) which will impact taking part in the study. (Note: participants may be allowed to attend if they have a condition/illness which would not interact with the active treatments or impede performance);
Have been diagnosed with or is undergoing treatment for a psychiatric disorder in the last 12 months;
Suffers from frequent migraines that require medication (more than or equal to 1 per month);
Have sleep disorders or are taking sleep aid medication;
Are pregnant, seeking to become pregnant or lactating;
Are currently taking prescription and over-the-counter (OTC) medications, especially contraceptive treatments for female participants, and those taken ‘as needed’ in the treatment of asthma (e.g. steroids) and hay fever (drowsy antihistamines only, e.g. chlorphenamine). (Note: There may be other instances of medication use where no interaction with the active treatments is likely, and which would not be expected to have any impact on brain function, participants may be able to attend the assessment);
Have taken antibiotics within the past 4 weeks (Note: participation is possible following a 4-week antibiotics washout prior to participating);
Smoke tobacco or vape nicotine or use nicotine replacement products
Have excessive caffeine intake (>500 mg per day), see CCQ;
Are self-evaluated to have difficulty in focusing on the tasks, e.g. too many meetings daily, etc.
Have a visual impairment that cannot be corrected with glasses or contact lenses (including color-blindness)
Refuse to consume the beverage containing artificial sweetener (e.g. aspartame, sucralose, acesulfame potassium, etc.).

### Example Beverages to be Evaluated for the Study

Various beverage products plus placebo are evaluated in the study. Table 1 shows the composition of example beverages used in the sage study. Table 2 shows the composition of example beverages in the grape seed study. Beverage products are non-commercialized carbonated beverage. All the ingredients are food grades and generally recognized as safe (GRAS) by FDA. The ingredients presented in the vehicle are at the same levels across all the products. Considering the study is double blinded, no ingredients information will be provided on the labels. Product label will read as follows: Sparkling Beverage, Code Number, Date of Manufacture, Not For Sale.

TABLE 1
Example beverages containing sage products for study 1 and 2.

| Sage study | Beverage | Ingredients |
|---|---|---|
| Example | Sage | Sage component (250 mg) + Eucalyptus oil |
| Beverage 1 | system 1 | (“Sage component” = aqueous sage |
| | | extract + sage oil) |
| Example | Sage | Sage component (250 mg) + Eucalyptus |
| Beverage 2 | system 2 | oil + tea polyphenol |
| | | (“Sage component” = aqueous sage |
| | | extract + sage oil) |
| Comparative | Sage | Sage component (250 mg) (“Sage |
| Example | | component” = aqueous sage |
| Beverage 1 | | extract + sage oil) |
| Placebo | vehicle | One serving contains vehicle without sage |
| | | component. |

TABLE 2
Example beverages containing grape seed product for study 3.

| Grape study | Beverage | Ingredients |
|---|---|---|
| Example | Grape seed system | Grape seed extract (150 mg) + |
| Beverage 3 | 1 (150 mg) | Eucalyptus oil |
| Example | Grape seed system | Grape seed extract (300 mg) + |
| Beverage 4 | 2 (300 mg) | Eucalyptus oil |
| Comparative | Sage | Aqueous Sage extract only (250 mg) |
| Example | | |
| Beverage 1 | | |
| Placebo | vehicle | One serving contains vehicle |
| (vehicle) | | without sage component or grape |
| | | seed extract. |

Participants are randomly allocated to a counterbalancing schedule dictating the order in which they receive the four interventions.

Participants are instructed to consume one of the four interventions during each of their assessment days (Day 1/Day 2/Day 3/Day 4), with the order of the interventions counterbalanced across the participants (via random allocation to a counterbalancing schedule—example above).

A 3-digit code is assigned to each intervention. A computerized counterbalancing schedule is generated by a third-party who has no other involvement in the study. The beverages are prepared and pre-packaged in a GMP pilot plant by a third-party who has no other involvement in the study as well, according to the counterbalancing schedule. The packaging/label only shows “Sparkling Beverage, Code Number, Date of Manufacture, and Not For Sale”. The beverages are stored at 4 °C till the time of consumption.

The use of any prescribed medicinal products (with exceptions as per exclusion criteria) are not allowed during the trial, including OTC products.

### Observations

### Efficacy

This is intended as an exploratory placebo-controlled study investigating the acute effects of selected products listed in tables 1-2 on cognitive performance and alertness in comparison to placebo. The efficacy is assessed with respect to the investigational products' comparative (to placebo) effects following a single dose on the following measures:

Cognitive function—executive function, attention, working memory, long-term memory (pre-dose baseline and 60 min post-dose).

Cognitive function and mental fatigue during extended performance of cognitively demanding tasks (pre-dose baseline and 60 min post-dose).

Alertness, stress and tranquility, as assessed by the Visual Analogue Mood Scales (VAMS) at 60 min post-dose.

### Safety

All the products are manufactured in a GMP pilot plant and cleared with microbial tests.

The ingredients of the beverages are not associated with any significant deleterious adverse effects. Therefore, the proposed doses of coffee berry juice for the beverage product combination should be safe. There is no subject autonomy concern. However, participants are encouraged to report any unusual or adverse effects during the study.

### Investigation

The study follows a randomized, double-blind, placebo-controlled, balanced cross-over design.

During the study, participants attend an introductory/training session and four active testing days (Day 1, Day 2, Day 3, Day 4), with no less than 1 day (24 hours) wash-out time between testing days.

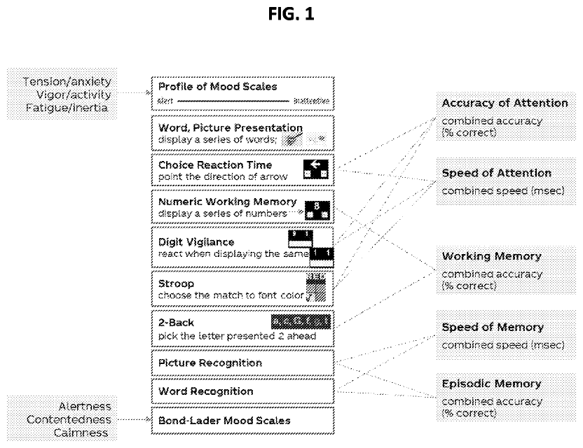

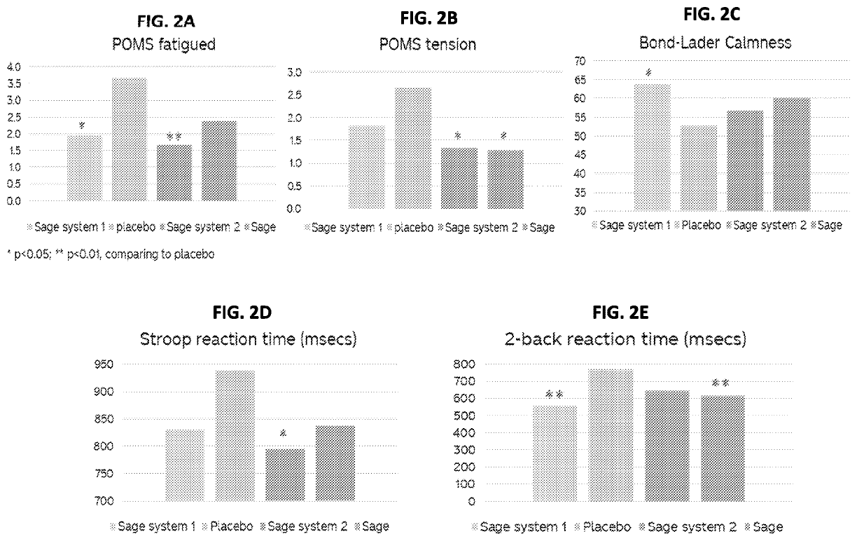

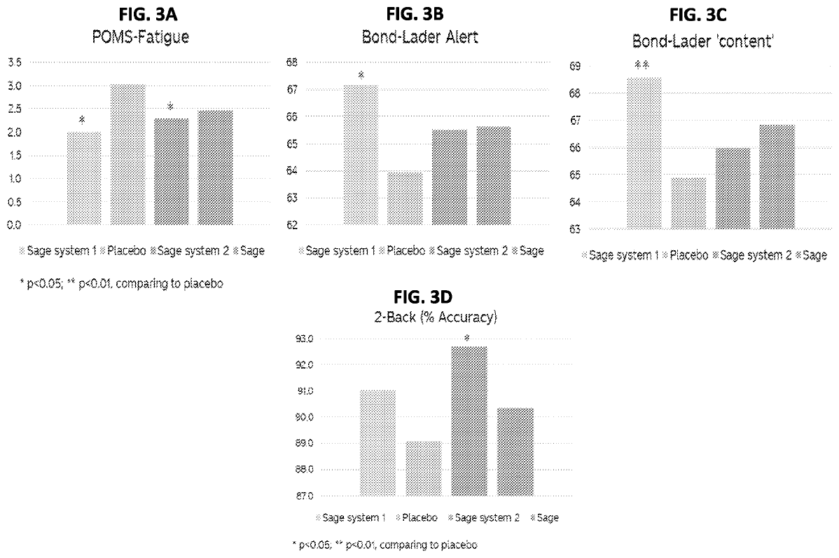

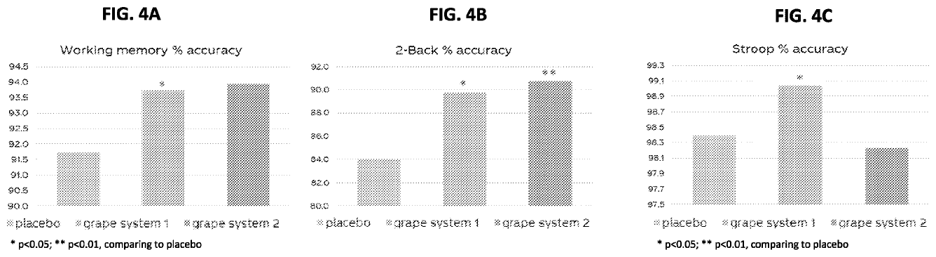

The Introductory session comprises the acquisition of Informed Consent Form (ICF), training and practice on the Cognimapp. The methodology on Days 1, 2, 3, and 4 are identical, with the exception that participants consume a different treatment during each testing day. On the testing day, participants are required to be abstained from alcohol (24 hr) and caffeine (18 hr). At least one hour after finishing the meal, participants are instructed to complete a 15-minute online cognitive and mood assessment on Cognimapp. An example of task assignment using Cognimapp is illustrated in FIG. 1. After the first cognitive assessment participants take their treatment (300 mL of beverage, be completed within 20 min) for the day and undergo cognitive/mood assessments identical to the above at 60 minutes post-dose. No additional food and drink other than water is allowed during assessments.

### Adherence to Protocol

An individual participant is to be withdrawn from the trial if:
The participant withdraws their consent, without need to justify the decision;
The participant no longer conforms to the inclusion/exclusion criteria;
The participant has to take any concomitant prescription drugs or any other substances the consumption of which would constitute grounds for exclusion (as per exclusion criteria) and the participant cannot be rescheduled;
The participant is no longer able to participate for other medical reasons (including adverse effects).

### Statistics

### Planned Analysis

Given that this is an exploratory investigation, with no existing data on which to base any predictions of treatment effects, no primary endpoints will be identified.

The intended analysis/analyses are applied to the following data (using pre-treatment data either to baseline adjust post-dose data.
Mood measures—Alertness, Stress, Tranquillity scores (pre-dose baseline and 60 min post-dose)
Cognitive function—Individual task parameters (pre-dose baseline and 60 min post-dose)
Cognitive function—composite cognitive factors (pre-dose baseline and 60 min post-dose)

Cognitive function and mental fatigue during extended performance of cognitively demanding tasks (pre-dose baseline and 60 min post-dose).

### Randomization

During the introductory visit participants are randomly allocated to receive their six treatments (as 3-digit product code) according to a blind counterbalancing schedule using a computer-generated random number list.

### Sample Size

The cross-over design and suggested sample size of 32 participants delivers good power (in excess of 90%) to detect the medium effect sizes seen previously with similar interventions. Power was calculated using G*Power 3.0.

### Results

Subjective measures of profile of mood scale and bond-lader mood scale were used to evaluate stress, vigorous, fatigue, alertness, contentedness, and calmness. The cognitive measures can be summarized as accuracy of attention, speed of attention, working memory, speed of memory, and episodic memory (FIG. 1). All the tasks listed in FIG. 1 produce multiple outcome measures. Each cognitive function measure was analysed using 60 min post-dose adjusted by pre-dose measurement as a covariate.

Three separate studies are performed to investigate sage systems (study 1 and 2) and grape seed system (study 3). Results are summarized below.

### Study 1 (Sage Systems; a Total of 9 Participants)

One participant was withdrawn from the study, totally 9 datasets were entered for analysis. As shown in Table 3, sage system 1 significantly reduces fatigue (p=0.022), improve calmness (p=0.034), 2-back reaction time (p=0.0001) against placebo; sage system 2 significantly reduced fatigue (p=0.014) and tension (p=0.022), improved stroop reaction time (p=0.046) and 2-back reaction time (n=0.021).

### Study 2 (Sage Systems; a Total of 36 Participants)

Eight participants were withdrawn from the study, totally 36 datasets were entered for analysis. As shown in Table 4, sage system 1 significantly reduction of fatigue (p=0.022), improvement of alertness (p=0.02) and contentedness (p=0.002) against placebo; sage system 2 displayed significance in reduction of fatigue (p=0.038), and improvement of accuracy of 2-back (p=0.037) against placebo.

### Study 3 (Grape Seed System; a Total of 30 Participants)

Ten participants were withdrawn from the study, totally 30 datasets were entered for analysis. As shown in Table 5, grape seed system 1 displayed significance at the compositive level of working memory (p=0.027), accuracy of 2-back (p=0.014) and stroop (p=0.028) against placebo; grape seed system 2 significantly improved accuracy of 2-back (0.01).

The above results provide evidence to support that polyphenol and at least one essential oil(s) compound when administered in combination with an optimal ratio and dosage have synergistic effect on reducing mental fatigue and tension, improving the accuracy of attention, working memory and alertness of a subject.

TABLE 3
Results observed from Studies 1

| Benefits | Sage system 1 | Sage system 2 | Sage |
|---|---|---|---|
| reduced fatigue | * | * | — |
| reduced tension | — | * | * |
| Bond-Lader calmness | * | — | — |
| Stroop reaction time | — | * | — |
| 2-back reaction time | ** | * | ** |

comparing to placebo,
*p < 0.05,
**p < 0.01

TABLE 4
Results observed from Studies 2

| Benefits | Sage system 1 | Sage system 2 | Sage |
|---|---|---|---|
| reduced fatigue | * | * | — |
| Bond-Lader alert | * | — | — |
| Bond-Lader content | ** | — | — |
| 2-back accuracy | — | * | — |

comparing to placebo,
*p < 0.05,
**p < 0.01

TABLE 5
Results observed from Studies 3

| Benefits | Grape seed system 1 | Grape seed system 2 | Sage |
|---|---|---|---|
| Working memory | * | — | — |
| 2-back accuracy | * | ** | * |
| Stroop accuracy | * | — | — |

comparing to placebo,
*p < 0.05,
**p < 0.01

### Example 2—Study on Acute Effects of Three Botanical Extracts on Cognitive Function and Mood in Healthy Young Adults

### Background and Objective

Several botanical extracts are reportedly beneficial to human cognition and mood, including sage, tea, coffee bean, and caffeine. However, their acute effects in cognitive function improvement and mood are not well known.

The aim of the proposed randomized, double-blind, placebo-controlled, balanced cross-over methodology is to assess the acute effects of composition systems having a selected botanical extract and caffeine on cognitive and mood function. The trial utilized a 60-minute computerized cognitive assessment (COMPASS-including the Cognitive Demand Battery, Visual Analogue Mood Scales (VAMS), Profile of Mood States (POMS), positive items from the Positive and Negative Affect Scale (PANAS), and a series of novel positive mood VAS). The cognitive/mood assessments will take place pre-dose and at 60 minutes, 180 minutes, and 300 minutes post-dose.

### Study Population

Four studies were conducted with total participants of 50 for each of the four studies. Respondents were selected following a remote screening that was completed via video/phone call.

The following criteria are used to selected participants for the study:
Participants are volunteering for the trials;
Participants must self-assess themselves as being in good health, including completing a health screening;
Aged 18 to 45 years at the time of giving consent;
Completion of a Caffeine Consumption Questionnaire (CCQ);
Consent to complete physiological eligibility measures, including (blood pressure, height and weight, and waist-to-hip ratio);
Complete training on cognitive and mood measures;
Participants agree to stay engaged in the tasks across all tests, which can be identified from the dataset.

Participants are not eligible to take part if they:
Have any pre-existing medical condition/illness (e.g., feel sick, have reduced brain performance, such as focus, memory, accuracy) which will impact taking part in the study. (Note: participants may be allowed to attend if they have a condition/illness which would not interact with the active treatments or impede performance);
Have been diagnosed with or is undergoing treatment for a psychiatric disorder in the last 12 months;
Suffers from frequent migraines that require medication (more than or equal to 1 per month);
Have sleep disorders or are taking sleep aid medication;
Are pregnant, seeking to become pregnant or lactating;
Are currently taking prescription and over the counter (OTC) medications, especially contraceptive treatments for female participants, and those taken ‘as needed’ in the treatment of asthma (e.g. steroids) and hay fever (drowsy antihistamines only, e.g. chlorphenamine). (Note: There may be other instances of medication use where no interaction with the active treatments is likely, and which would not be expected to have any impact on brain function, participants may be able to attend the assessment);
Have taken antibiotics within the past 4 weeks (Note: participation is possible following a 4-week antibiotics washout prior to participating);
Smoke tobacco or vape nicotine or use nicotine replacement products
Have excessive caffeine intake (>500 mg per day), see CCQ;
Are self-evaluated to have difficulty in focusing on the tasks, e.g. too many meetings daily, etc.
Have a visual impairment that cannot be corrected with glasses or contact lenses (including color-blindness)
Refuse to consume the beverage containing artificial sweetener (e.g. aspartame, sucralose, acesulfame potassium, etc.).
   The demographic information for the 50 participants included in the analysis are shown in table 6. Participant dispositions are shown below in FIG. 5.

TABLE 6
Demographic Information

| Number of participants | 50 | |
|---|---|---|
| Age | 27.9 | years |
| Age range | 18-45 | years |
| Gender | F 30/M 20 | |
| Years in Education | 16.8 | years |
| Portions of Fruit & Veg/day | 3.5 | |
| Caffeine | 118 | mg/day |
| Weight | 68.3 | kg |
| Height | 167.6 | cm |

| | |
|---|---|
| BMI | 24.1 |

### Example Beverages to be Evaluated for the Study

Various beverage products plus placebo are evaluated in the study. Table 7 shows the composition of example beverages used in the study. All the ingredients are food grades and generally recognized as safe (GRAS) by FDA. The ingredients presented in the vehicle are at the same levels across all the products. Considering the study is double blinded, no ingredients information will be provided on the labels.

TABLE 7
Example beverages for studies one through four

| Botanical study | Beverage | Ingredients |
|---|---|---|
| Example | Tea Extract | 50 mg caffeine + 80 mg tea extract (>94% EGCG) |
| Beverage 1 | (“Tea + Caff”) | |
| Example | Sage Extract | 50 mg caffeine + 100 mg sage extract |
| Beverage 2 | (“Sage + Caff”) | |
| Comparative | Coffee Bean Extract | 200 mg coffee bean extract (w 2% caffeine) + |
| Example | (“Coff + Caff”) | caffeine match to 50 mg totally |
| Beverage 3 | | |
| Placebo | Flavor Vehicle | One serving contains vehicle without a botanical |
| | (“placebo”) | component. |

Each of the four beverages were bottled and identified by the manufacturers own code which was site specific (A, B, C, D). The order in which the participants received the four beverages was counterbalanced across the group via random allocation to counterbalancing schedule.

The beverages were each 10 oz and decanted into individual opaque, lidded containers on a daily basis. Each beverage was consumed in the presence of the investigator, ensuring 100% compliance.

### Procedure

Participants underwent initial online screening, followed by an in-person lifestyle and demographic screening. Prior to beginning the study, participants completed a short training session where they performed cognitive tasks, in the form of three repetitions of shortened versions of the COMPASS cognitive tasks, followed by the completion of the full-length, 60-minute task.

The methodology of each of the four studies that followed were identical, with the exception that each participant consumed a different beverage during each of their four visits. Participants were required to abstain from alcohol for (24) hours, and from caffeine overnight. Upon arrival each day, participants completed a 60-minute COMPASS assessment. Thereafter, participants would take their treatment for the day and underwent COMPASS assessments at the 60 minute mark, the 180 minute mark, and the 300 minute post-dose.

### Observations

### Efficacy

This is intended as an exploratory placebo-controlled study investigating the acute effects of selected products listed in table 7 on cognitive performance and alertness, as well as mood, in comparison to placebo. The efficacy is assessed with respect to the investigational products' comparative (to placebo) effects following a single dose on the following measures:

### Safety

All the products are manufactured in a GMP pilot plant and cleared with microbial tests.

The ingredients of the beverages are not associated with any significant deleterious adverse effects. There is no subject autonomy concern. However, participants are encouraged to report any unusual or adverse effects during the study.

### Investigation

The study follows a randomized, double-blind, placebo-controlled, balanced cross-over design.

During the study, participants attend an introductory/training session and four active testing days (Day 1, Day 2, Day 3, Day 4), with no less than 1 day (24 hours) wash-out time between testing days.

The methodology on Days 1, 2, 3, and 4 are identical, with the exception that participants consume a different treatment during each testing day. On the testing day, participants are required to be abstained from alcohol (24 hr) and caffeine (overnight). No additional food and drink other than water is allowed during assessments.

### Adherence to Protocol

An individual participant is to be withdrawn from the trial if:
The participant withdraws their consent, without need to justify the decision;
The participant no longer conforms to the inclusion/exclusion criteria;
The participant has to take any concomitant prescription drugs or any other substances the consumption of which would constitute grounds for exclusion (as per exclusion criteria) and the participant cannot be rescheduled;
The participant is no longer able to participate for other medical reasons (including adverse effects).

### Cognitive and Mood Assessment

Several individual cognitive/mood assessment tasks were used. Some of the tasks employed were standard ‘classic’ computerized cognitive tasks, and ‘Cognitive Demand Battery’ (CBD) tasks were also used (FIG. 6).

### Cognitive Outcomes: Derivation of Global Outcomes and Cognitive Factors

Global performance measures and cognitive factors derived from the COMPASS were used to determine whether the treatment had a global effect across the entire cognitive battery, or an effect restricted to a cognitive domain.

### ‘Speed of Performance’:

This global outcome measures overall speed of performance. It is calculated as the average speed of performance (msec) of all of the tasks within the battery that collect reaction time data to individual stimuli: Choice Reaction Time, Simple Reaction Time, Digit Vigilance Task, Rapid Visual Information Processing (average of 3 repetitions), Numeric Working Memory, Delayed Picture Recognition, Delayed Word Recognition.

### ‘Accuracy of Performance’:

Calculated as the average percent of accuracy of all of the tasks for which data may be collected including: choice reaction time, digit vigilance task, rapid visual information processing, numeric working memory, corsi blocks, immediate word recall, delayed word recall, delayed picture recognition, and delayed word recognition.

### ‘Speed of Attention’:

Calculated as the average speed (msecs) of performing the attention tasks within the battery.

### ‘Accuracy of Attention’:

Calculation as the average percent of accuracy of performing the attention tasks within the battery.

### ‘Working Memory’:

Calculated as the average percent accuracy/maximum score of performing the working memory tasks within the battery.

### ‘Episodic Memory’:

Calculated as the average percent correct of the long-term memory tasks within the battery.

### ‘Speed of Memory’:

Calculated as the average speed (msec) of performing the working and episodic memory tasks that collect reaction time data to individual stimuli.

### Cognitive Demand Battery (CBD):

The objective of this battery is to assess the impact of treatment on speed/accuracy and mental fatigue during continuous performance of cognitively demanding tasks. The battery comprises the performance of a 10 min battery of mentally challenging tasks, which are repeated three times in immediate succession.

### Mood Measures:

Profile of Mood State (POMS) is a 37-item inventory. Individual item scores are organized into six dimensions of mood: anger, confusion, depression, fatigue, tension, and vigour.

Visual Analogue Mood Scales (VAMS) are completed as part of the COMPASS battery. They comprise eighteen visual analogue scales in pairs of antonymic mood/state adjectives. Participants rate where they would position themselves within the two adjectives, in the moment.

Positive and Negative Affect Schedule (PANAS) is a self-report questionnaire of two ten-item scales that measure positive and negative affect, respectively.

### Statistics

### Planned Analysis

No primary or secondary outcomes were delineated, but each of the cognitive outcomes followed a natural hierarchy, taking precedence over each of the individual task scores.

### Analysis

Prior to primary analysis of the effects of treatment, pre-dose differences between treatments were investigated by one-way ANOVAS, or in the case of Cognitive Demand Battery (CBD) outcomes, two-way (treatment×repetition) ANOVAS.

For all cognitive and mood measures, the primary analysis of post-dose data was by Linear Mixed Models (LMM) using the MIXED procedure in SPSS with pre-dose baseline for each outcome included as a covariate. For cognitive global performance, cognitive factor and individual task outcomes derived from COMPASS, and the mood outcomes, terms were fitted for the drink (A, B, C, D), and assessment (60, 180, and 300 min post-dose) and their interaction.

In the event of a significant main effect of drink or drink x assessment interaction effect, a single set of a priori planned comparisons were undertaken. Any main effects of product were explored by comparing all three active interventions (Tea+Caff, Sage+Caff, Coff+Caff) to placebo.

### Randomization

During the introductory visit participants are randomly allocated to receive their four treatments (as single-digit product code) according to a blind counterbalancing schedule using a computer-generated random number list.

### Sample Size

The cross-over design and suggested sample size of 48 participants delivers good power (in excess of 95%) to detect a genuine treatment related effect. Power was calculated using G*Power 3.0.

### Results

### Cognitive Function

The primary analysis (LMM) of the global speed and accuracy outcomes showed that there was a significant main effect of the interventions on the global ‘Accuracy of Performance’ measure. Reference to the comparisons of averaged data across post-dose time-points showed overall accuracy was improved following Coff+Caff (P<0.001) and Sage+Caff (P=0.01) in comparison to placebo (Table 8). With respect to the comparisons of the data from the individual assessments, Coff+Caff was associated with significant accuracy benefits during the 180 min and 300 min assessments (p<0.01), whereas Sage+Caff was only associated with improvements during the last assessment (p<0.05) in comparison to placebo.

The primary analysis also showed a main effect of the drinks on both the Speed of Attention and Accuracy of Attention factors derived from the COMPASS tasks. In the case of Speed of Attention, benefits were only seen across assessments following Coff+Caff (p<0.01), with a trend towards the same effect for Sage+Caff (p<0.01). In line with this, speed was only improved during one individual assessment (180 min) and only following Coff+Caff (p<0.01). However, all three drinks were associated with improved Accuracy of Attention across assessments in comparison to placebo (Tea+Caff, p<0.05; Sage+Caff and Coff+Caff, p<0.001, Table 8). With respect to the individual assessments, all three drinks resulted in better accuracy than placebo during the 60 min assessment (Tea+Caff/Sage+Caff, p<0.05; Coff+Caff, p<0.001) and both Sage+Caff and Coff+Caff improved performance during the 180 min (both p<0.01) and 300 min (both p<0.05) assessments. There was also a trend towards a main effect of the drinks on Episodic Memory F (3, 215)=2.117, p<0.1], with this predicated on significantly improved memory performance following Coff+Caff (p<0.05) in comparison to placebo across assessments.

There was no significant effect of the drinks on the remaining global measure and the other cognitive factors.

The initial LMM analysis showed a main effect suggesting drink related improvements to the performance of all three tasks within the Cognitive Demand Battery. All three drinks were associated with better performance across the testing period in terms of more correct subtractions on the Serial 3s task in comparison to placebo (Tea+Caff and Sage+Caff, p<0.01; Coff+Caff, p, 0.001, Table 8). Reference to comparisons of the individual assessment data showed that this relationship reached significance for Coff+Caff at all three time points (60 min, p<0.05; 180 min, p<0.01; 300 min, p<0.05), for Tea+Caff at the later two time points (180, p<0.05; 300, p<0.01) and for Sage+Caff during the last assessment (300, p<0.05). There was also a main effect of drink on the number of incorrect Serial 3s subtractions [F (3, 1642)=4.35, p<0.01). All three drinks also resulted in improved accuracy of performing the Rapid Visual Information Processing (RVIP) task across post-dose assessments in comparison to placebo (Tea+Caff, p<0.01; Sage+Caff and Coff+Caff, p<0.001). Both Sage+Caff and Coff+Caff resulted in improvements during each of the three post-dose assessments (60 min-Sage+Caff, p<0.05; Coff+Caff, p<0.001:180 min-both p<0.01:300 min-both p<0.001) whereas Tea+Caff only resulted in improved performance during the 60 min and 300 min assessments (both p<0.05).

### Conclusion:

In comparison to placebo, all three beverages were associated with improved Accuracy of Attention, and improvements in the performance of the Serial 3s and RVIP tasks, alongside reductions in mental fatigue during performance of the CDB tasks. Both Sage+Caff and Coff+Caff also resulted in significantly improved performance accuracy across all of the cognitive tasks (Accuracy of Performance). Coff+Caff alone significantly improved the speed of performing the attention tasks (Speed of Attention), the performance of long-term memory tasks (Episodic Memory) and performance of the Serial 7s task (Table 8).

TABLE 8
Results observed from the study in Example 2

| Benefits | Tea + Caff | Sage + Caff | Coff + Caff |
|---|---|---|---|
| Accuracy of performance | — | ** | *** |
| Speed of attention | — | — | * |
| Accuracy of attention | * | *** | *** |
| Episodic memory | — | — | * |
| Serial 3 s correct ↑ | ** | ** | *** |
| Serial 3 s error ↓ | — | — | ** |
| Serial 7 s correct ↑ | — | — | *** |
| RVIP accuracy ↑ | ** | *** | *** |
| RVIP speed ↑ | — | — | ** |
| Mental fatigue ↓ | *** | ** | *** |

Comparing to placebo,
* p < 0.05;
** p < 0.01;
*** p < 0.001

### Example 3—Study on Acute Effects of Three Botanical Extracts on Cognitive Function and Mood in Healthy Young Adults

### Background and Objective

Several botanical extracts are reportedly beneficial to human cognition and mood, including sage, tea, lemon balm, and chamomile. However, their acute effects in cognitive function improvement and mood are not well known.

The aim of the proposed randomized, double-blind, placebo-controlled, balanced cross-over methodology is to assess the acute effects of composition systems having i) sage and tea extracts, ii) lemon balm extract, iii) chamomile extract, and iv) a placebo on cognitive and mood function. The trial utilized a 60-minute computerized cognitive assessment (COMPASS—including the Cognitive Demand Battery, Visual Analogue Mood Scales (VAMS), Profile of Mood States (POMS), positive items from the Positive and Negative Affect Scale (PANAS), and a series of novel positive mood VAS). The cognitive/mood assessments will take place pre-dose and at 60 minutes, 180 minutes, and 300 minutes post-dose.

### Study Population

Four studies were conducted with total participants of 50 for each of the four studies. Respondents were selected following a remote screening that was completed via video/phone call.

The following criteria are used to selected participants for the study:
Participants are volunteering for the trials;
Participants must self-assess themselves as being in good health, including completing a health screening;
Aged 18 to 45 years at the time of giving consent;
Completion of a Caffeine Consumption Questionnaire (CCQ);
Consent to complete physiological eligibility measures, including (blood pressure, height and weight, and waist-to-hip ratio);
Complete training on cognitive and mood measures;
Participants agree to stay engaged in the tasks across all tests, which can be identified from the dataset.

Participants are not eligible to take part if they:
Have any pre-existing medical condition/illness (e.g., feel sick, have reduced brain performance, such as focus, memory, accuracy) which will impact taking part in the study. (Note: participants may be allowed to attend if they have a condition/illness which would not interact with the active treatments or impede performance);
Have been diagnosed with or is undergoing treatment for a psychiatric disorder in the last 12 months;
Suffers from frequent migraines that require medication (more than or equal to 1 per month);
Have sleep disorders or are taking sleep aid medication;
Are pregnant, seeking to become pregnant or lactating;
Are currently taking prescription and over-the-counter (OTC) medications, especially contraceptive treatments for female participants, and those taken ‘as needed’ in the treatment of asthma (e.g. steroids) and hay fever (drowsy antihistamines only, e.g. chlorphenamine). (Note: There may be other instances of medication use where no interaction with the active treatments is likely, and which would not be expected to have any impact on brain function, participants may be able to attend the assessment);
Have taken antibiotics within the past 4 weeks (Note: participation is possible following a 4-week antibiotics washout prior to participating);
Smoke tobacco or vape nicotine or use nicotine replacement products
Have excessive caffeine intake (>500 mg per day), see CCQ;
Are self-evaluated to have difficulty in focusing on the tasks, e.g. too many meetings daily, etc.
Have a visual impairment that cannot be corrected with glasses or contact lenses (including color-blindness)
Refuse to consume the beverage containing artificial sweetener (e.g. aspartame, sucralose, acesulfame potassium, etc.).
   The demographic information for the 50 participants included in the analysis are shown in table 1. Participant dispositions are shown below.

TABLE 9
Demographic Information

| Number of participants | 50 | |
|---|---|---|
| Age | 29.5 | years |
| Age range | 18-45 | years |
| Gender | F 34/M 16 | |
| Years in Education | 16.4 | years |
| Portions of Fruit & Veg/day | 3.5 | |
| Caffeine | 107 | mg/day |
| Weight | 68.6 | kg |
| Height | 169 | cm |

| | |
|---|---|
| BMI | 23.9 |

### Example Beverages to be Evaluated for the Study

Various beverage products plus placebo are evaluated in the study. Table 10 shows the composition of example beverages used in the study.

TABLE 10
Example beverages used for the study

| Botanical study | Beverage | Ingredients |
|---|---|---|
| Example | Green Tea | (1.7 g) concentrated green tea infusion + (300 mg) white |
| Beverage 1 | Infusion | tea extract + (333 mg) sage extract |
| Example | Lemon Balm | (300 mg) lemon balm extract |
| Beverage 2 | Extract | |
| Comparative | Chamomile | (250 mg) chamomile extract |
| Example | Extract | |
| Beverage 3 | | |
| Placebo | vehicle | One serving contains vehicle without a botanical |
| | | component. |

Each of the four beverages were bottled and identified by the manufacturers own code which was site specific (A, B, C, D). The order in which the participants received the four beverages was counterbalanced across the group via random allocation to counterbalancing schedule.

The beverages were each 300 ml and decanted into individual opaque, lidded containers on a daily basis. Each beverage was consumed in the presence of the investigator, ensuring 100% compliance.

### Procedure

Participants underwent initial online screening, followed by an in-person lifestyle and demographic screening. Prior to beginning the study, participants completed a short training session where they performed cognitive tasks, in the form of three repetitions of shortened versions of the COMPASS cognitive tasks, followed by the completion of the full-length, 60-minute task.

The methodology of each of the four studies that followed were identical, with the exception that each participant consumed a different beverage during each of their four visits. Participants were required to abstain from alcohol for (24) hours, and from caffeine overnight. Upon arrival each day, participants completed a 60-minute COMPASS assessment. Thereafter, participants would take their treatment for the day and underwent COMPASS assessments at the 60 minute mark, the 180 minute mark, and the 300 minute post-dose.

### Observations

### Efficacy

This is intended as an exploratory placebo-controlled study investigating the acute effects of selected products listed in table 1 on cognitive performance and alertness, as well as mood in comparison to placebo. The efficacy is assessed with respect to the investigational products' comparative (to placebo) effects following a single dose on the following measures:

COMPASS (including the Cognitive Demand Battery, Visual Analogue Mood Scales (VAMS), Profile of Mood Scales (POMS), positive items from the Positive and Negative Affect Scale [PANAS], and bespoke stress (Stress VAS) and positive mood (Positive VAS) visual analogue scales). After the first cognitive assessment participants took their treatment for the day and underwent cognitive/mood assessments identical to the above starting at 60 min, 180 min and 300 min post-dose.

### Safety

All the products are manufactured in a GMP pilot plant and cleared with microbial tests.

The ingredients of the beverages are not associated with any significant deleterious adverse effects. Therefore, the proposed doses of botanical extracts for the beverage product combination should be safe. There is no subject autonomy concern. However, participants are encouraged to report any unusual or adverse effects during the study.

### Investigation

The study follows a randomized, double-blind, placebo-controlled, balanced cross-over design.

During the study, participants attend an introductory/training session and four active testing days (Day 1, Day 2, Day 3, Day 4), with no less than 1 day (24 hours) wash-out time between testing days.

The methodology on Days 1, 2, 3, and 4 are identical, with the exception that participants consume a different treatment during each testing day. On the testing day, participants are required to be abstained from alcohol (24 hr) and caffeine (overnight). No additional food and drink other than water is allowed during assessments.

### Adherence to Protocol

An individual participant is to be withdrawn from the trial if:
The participant withdraws their consent, without need to justify the decision;
The participant no longer conforms to the inclusion/exclusion criteria;
The participant has to take any concomitant prescription drugs or any other substances the consumption of which would constitute grounds for exclusion (as per exclusion criteria) and the participant cannot be rescheduled;
The participant is no longer able to participate for other medical reasons (including adverse effects).

### Cognitive and Mood Assessment

Several individual cognitive/mood assessment tasks were used. Some of the tasks employed were standard ‘classic’ computerized cognitive tasks, and ‘Cognitive Demand Battery’ (CBD) tasks were also used (FIG. 7).

### Cognitive Outcomes: Derivation of Global Outcomes and Cognitive Factors

Global performance measures and cognitive factors derived from the COMPASS were used to determine whether the treatment had a global effect across the entire cognitive battery, or an effect restricted to a cognitive domain.

### ‘Speed of Performance’:

This global outcome measures overall speed of performance. It is calculated as the average speed of performance (msec) of all of the tasks within the battery that collect reaction time data to individual stimuli: Choice Reaction Time, Simple Reaction Time, Digit Vigilance Task, Rapid Visual Information Processing (average of 3 repetitions), Numeric Working Memory, Delayed Picture Recognition, Delayed Word Recognition.

### ‘Accuracy of Performance’:

Calculated as the average percent of accuracy of all of the tasks for which data may be collected including: choice reaction time, digit vigilance task, rapid visual information processing, numeric working memory, corsi blocks, immediate word recall, delayed word recall, delayed picture recognition, and delayed word recognition.

### ‘Speed of Attention’:

Calculated as the average speed (msecs) of performing the attention tasks within the battery.

### ‘Accuracy of Attention’:

Calculation as the average percent of accuracy of performing the attention tasks within the battery.

### ‘Working Memory’:

Calculated as the average percent accuracy/maximum score of performing the working memory tasks within the battery.

### ‘Episodic Memory’:

Calculated as the average percent correct of the long-term memory tasks within the battery.

### ‘Speed of Memory’:

Calculated as the average speed (msec) of performing the working and episodic memory tasks that collect reaction time data to individual stimuli.

### Cognitive Demand Battery (CBD):

The objective of this battery is to assess the impact of treatment on speed/accuracy and mental fatigue during continuous performance of cognitively demanding tasks. The battery comprises the performance of a 10 min battery of mentally challenging tasks, which are repeated three times in immediate succession.

### Mood Measures:

Profile of Mood State (POMS) is a 37-item inventory. Individual item scores are organized into six dimensions of mood: anger, confusion, depression, fatigue, tension, and vigour.

Visual Analogue Mood Scales (VAMS) are completed as part of the COMPASS battery. They comprise eighteen visual analogue scales in pairs of antonymic mood/state adjectives. Participants rate where they would position themselves within the two adjectives, in the moment.

Positive and Negative Affect Schedule (PANAS) is a self-report questionnaire of two ten-item scales that measure positive and negative affect, respectively.

### Statistics

### Planned Analysis

No primary or secondary outcomes were delineated, but each of the cognitive outcomes followed a natural hierarchy, taking precedence over each of the individual task scores.

### Analysis

Prior to primary analysis of the effects of treatment, pre-dose differences between treatments were investigated by one-way ANOVAS, or in the case of Cognitive Demand Battery (CBD) outcomes, two-way (treatment×repetition) ANOVAS.

For all cognitive and mood measures, the primary analysis of post-dose data was by Linear Mixed Models (LMM) using the MIXED procedure in SPSS with pre-dose baseline for each outcome included as a covariate. For cognitive global performance, cognitive factor and individual task outcomes derived from COMPASS, and the mood outcomes, terms were fitted for the drink (A, B, C, D), and assessment (60, 180, and 300 min post-dose) and their interaction.

In the event of a significant main effect of drink or drink x assessment interaction effect, a single set of a priori planned comparisons were undertaken. Any main effects of product were explored by comparing all three active interventions (sage+tea, lemon balm, chamomile) to placebo.

### Randomization

During the introductory visit participants are randomly allocated to receive their four treatments (as single-digit product code) according to a blind counterbalancing schedule using a computer-generated random number list.

### Sample Size

The cross-over design and suggested sample size of 48 participants delivers good power (in excess of 95%) to detect a genuine treatment related effect. Power was calculated using G*Power 3.0.

### Results

There were no baseline differences on any cognitive or mood measures.

### Cognitive Function

The primary analysis (LMM) of the global speed and accuracy outcomes showed that there was a significant main effect of the interventions on the global ‘Accuracy of Performance’ measure. Reference to the comparisons of averaged data across post-dose time-points showed overall accuracy was improved following sage+tea (P<0.001) in comparison to placebo. Looking at the individual assessments, sage+tea also resulted in improved performance during the 180 min (p<0.01) and 300 min (p<0.05) assessments in comparison to placebo.

The primary analysis also showed a main effect of the drinks on the Accuracy of Attention and Speed of Attention. The sage+tea drink outperformed the placebo across assessments.

The initial LMM analysis showed a main effect of the drinks on performance of all three tasks within the CDB. In all instances reference to the planned comparisons of data across assessments for each of the above outcomes showed that performed was enhanced following sage+tea in comparison to placebo (all p<0.001). Looking at the individual assessment time-points, sage+tea also outperformed placebo at all three post-dose assessments on each outcome.

### Mood and Psychological State

The only VAMS factor to see a significant effect of the interventions on the initial analysis was the ‘Alertness’ outcome. On this measure participants rated themselves as more alert across assessments after the sage+tea drink than after the placebo drink (p<0.01), with the same effect seen during both the 180 min (p<0.01) and 300 min (p<0.05) individual assessments.

There were significant effects of the drinks on the (averaged) ‘stress’ VAS. Reference to planned comparisons of data across assessments showed that participants reported themselves to be more stressed (or less calm) than placebo (p<0.01) after the chamomile drink. The ‘Positive VAS’, representing overall positive mood/psychological state, also saw a significant effect of the drinks.

### Conclusion

In comparison to the placebo, a single serving of the sage+tea beverage resulted in broad improvements in both cognitive function and psychological state. In terms of cognitive function, the sage+tea drink resulted in improved accuracy across all of the tasks in the COMPASS battery (Accuracy of Attention), and specific improvements in both the accuracy (Accuracy of Attention) and speed of performance (Speed of Attention) of the attention tasks within the battery.

All of the compositions and methods disclosed and claimed herein can be made and executed without undue experimentation in light of the present disclosure. While the compositions and methods of this disclosure have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the disclosure. More specifically, it will be apparent that certain agents, additives, and ingredients that are similar according to their physical, chemical, physiological, and/or gustative properties may be substituted for the agents, additives and ingredients described herein while the same or similar results would be achieved. All such similar substitutes and modifications apparent to those skilled in the art are deemed to be within the spirit, scope, and concept of the disclosure as defined by the hereinafter appended claims.

The following numbered clauses define further example aspects and features of the present disclosure:
1. An edible composition for improving a condition of a human, the composition comprising:
   a first polyphenol; and
   at least one essential oil(s),
   wherein a ratio of total polyphenol to total essential oil(s) is from about 1:10 to about 100:1.
2. The composition of clause 1, wherein the at least one essential oil(s), comprises:
   terpenes, monoterpenes, sesquiterpenes, diterpenes, esters, aldehydes, ketones, alcohols, phenols and oxides, and derivatives and/or any combinations thereof.
3. The composition of any one of clauses 1-2, wherein the first polyphenol and the at least one essential oil(s) are derived from the same plant species.
4. The composition of any one of clauses 1-2, wherein the first polyphenol and the at least one essential oils(s) are derived from different plants species.
5. The composition of any one of clauses 1-3, wherein the at least one essential oil(s) comprises,
   a first essential oil; and
   a second essential oil;
   wherein the first essential oil is derived from the same or different plant species as the first polyphenol, and the second essential oil is derived from a different plant species than the first essential oil.
6 The composition of any one of clauses 1-5, wherein the at least one essential oil(s) are derived from a plant genus selected from Rosa, Sophora, Citrus, Fragaria, Juniperus, Murraya, Sambucus, Syzygium, Angelica, Hibiscus, Helichrysum, Chamomilla, Chamaemelum, Cymbopogon, Geranium, Matricaria, Jasmimum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselinum, Pimpinella, Piper, Thymus, Zingiber, Humulus and/or any combinations thereof.
7. The composition of any one of claims 1-6, wherein the first polyphenol is derived from a plant genus selected from Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Schinus, Salvia, Satureia, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.
8. The composition of any one of clauses 1-7, wherein the first polyphenol is selected from a group comprising a flavonoid, a phenolic acid, a curcuminoid, a lignan, a stilbene, a phenolic terpene, a glycoside, a glucuronide, a depside and/or any combination thereof.
9. The composition of clause 8, wherein the flavonoid is selected from a group comprising a flavanol, a flavone, a flavonol, a flavanone, an isoflavone, an anthocyanidin, a gallate, a glycoside, a polymer thereof and any combination thereof.
10. The composition of clause 9, wherein the flavanol is selected from a group comprising a catechin, an epicatechin, a catechin gallate, an epicatechin gallate, a gallocatechin, an epigallocatechin, a gallocatechin gallate, an epigallocatechin gallate, a proanthocyanidin and any combinations thereof.
11. The composition of any one of clauses 9-10, wherein the flavone is selected from a group comprising a luteolin, an apigenin, a tangeritin, a chrysin, and any combination thereof.
12. The composition of any one of clauses 9-11, wherein the flavonol is selected from a group comprising a quercetin, a kaempferol, a myricetin, a rutin, and any combination thereof.
13. The composition of any one of clauses 9-12, wherein the flavanone is selected from a group comprising a eriodictyol, a hesperetin, a narigenin, and any combination thereof.
14. The composition of any one of clauses 9-13, wherein the isoflavone is selected from a group comprising a genistein, a daidzein and any combination thereof.
15. The composition of any one of clauses 9-14, wherein the anthocyanidin is selected from a group comprising a cyanidin, a delphinidin, a malvidin, a pelargonidin, an aaurantinidin, a capensinidin, an europinidin, a hirsutidin, a peonidin, a petunidin, a pulchellidin, a rosinidin and any combination thereof.
16. The composition of any one of clauses 8-15, wherein the phenolic acid is selected from a group comprising a rosmarinic acid, a vanillic acid, a caffeic acid, a gallic acid, a protocatechuic acid, a salicyclic acid, a ferulic acid, a sinapic acid, a chlorogenic acids, a coumaric acid and any combination thereof.
17 The composition of any one of clauses 8-16, wherein the curcuminoid is selected from a group comprising a bisdemethoxycurcumin, a demethoxycurcumin, a curcumin, and any combination thereof.
18. The composition of any one of clauses 8-17, wherein the lignan is selected from a group comprising a lariciresinol, a pinoresino, a matairesinol, a syrigaresinol, a sesamin, a sesaminol, and any combination thereof.
19. The composition of any one of clauses 8-18, wherein the stilbene is selected from a group comprising a resveratrol, a pterostilbene, and any combination thereof.
20. The composition of any one of clauses 8-19, wherein the phenolic terpene is selected from a group comprising a carnosic acid, a rosmanol, a carnosol, and any combination thereof.
21. The composition of any one of clauses 1-20, wherein the first polyphenol is derived from sage.
22 The composition of any one of clauses 1-21, wherein the first polyphenol is derived from Salvia officinalis and wherein the at least one essential oil(s) is derived from Salvia lavandulifolia.
23. The composition of any one of clauses 1-20, wherein the first polyphenol is derived from grape seed.
24. The composition of any one of clauses 1-23, further comprising a second polyphenol.
25. The composition of clause 24, wherein the second polyphenol is selected from a group comprising a flavonoid, a phenolic acid, a curcuminoid, a lignan, a stilbene, a phenolic terpene, a glycoside, a glucuronide, a depside and/or any combination thereof.
26. The composition of clause 25, wherein the flavonoid is selected from a group comprising a flavanol, a flavone, a flavonol, a flavanone, an isoflavone, an anthocyanidin, a gallate, a glycoside, a polymer thereof and any combination thereof.
27. The composition of clause 26, wherein the flavanol is selected from a group comprising a catechin, an epicatechin, a catechin gallate, an epicatechin gallate, a gallocatechin, an epigallocatechin, a gallocatechin gallate, an epigallocatechin gallate, a proanthocyanidin and any combinations thereof.
28. The composition of any one of clauses 26-27, wherein the flavone is selected from a group comprising a luteolin, an apigenin, a tangeritin, a chrysin, and any combination thereof.
29 The composition of any one of clauses 26-28, wherein the flavonol is selected from a group comprising a quercetin, a kaempferol, a myricetin, a rutin, and any combination thereof.
30. The composition of any one of clauses 26-29, wherein the flavanone is selected from a group comprising a eriodictyol, a hesperetin, a narigenin, and any combination thereof.
31. The composition of any one of clauses 26-30, wherein the isoflavone is selected from a group comprising a genistein, a daidzein and any combination thereof.
32. The composition of any one of clauses 26-31, wherein the anthocyanidin is selected from a group comprising a cyanidin, a delphinidin, a malvidin, a pelargonidin, an aaurantinidin, a capensinidin, an europinidin, a hirsutidin, a peonidin, a petunidin, a pulchellidin, a rosinidin and any combination thereof.
33. The composition of any one of clauses 26-32, wherein the phenolic acid is selected from a group comprising a rosmarinic acid, a vanillic acid, a caffeic acid, a gallic acid, a protocatechuic acid, a salicyclic acid, a ferulic acid, a sinapic acid, a chlorogenic acids, a coumaric acid and any combination thereof.
34 The composition of any one of clauses 26-33, wherein the curcuminoid is selected from a group comprising a bisdemethoxycurcumin, a demethoxycurcumin, a curcumin, and any combination thereof.
35. The composition of any one of clauses 26-34, wherein the lignan is selected from a group comprising a lariciresinol, a pinoresino, a matairesinol, a syrigaresinol, a sesamin, a sesaminol, and any combination thereof.
36 The composition of any one of clauses 26-35, wherein the stilbene is selected from a group comprising a resveratrol, a pterostilbene, and any combination thereof.
37. The composition of any one of clauses 26-36, wherein the phenolic terpene is selected from a group comprising a carnosic acid, a rosmanol, a carnosol, and any combination thereof.
38. The composition of any one of clauses 24-37, wherein the second polyphenol is derived from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof.
39 The composition of any one of clauses 24-37, wherein the second polyphenol is derived from a plant genus selected from Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.
40. The composition of any one of clauses 24-39, wherein the second polyphenol is not derived from sage or grape seed.
41. The composition of any one of clauses 1-40, further comprising a sweetener.
42. The composition of clause 41, wherein the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs), and combinations thereof.
43. The composition of any one of clauses 1-42, further comprising at least one additive or functional ingredient or both.
44. The composition of any one of clauses 1-43, wherein the composition is in a drinkable liquid form, a concentrate form, a dry form, or a semi-dry form.
45. The composition of any one of clauses 1-44, wherein the composition is a gum, gel, tablet, capsule, granule, cubic, or dry powder.
46. The composition of any one of clauses 1-45, wherein the composition is a beverage selected from the group of non-carbonated beverage, carbonated beverage, juice beverage, fruit juice, coffee beverage, tea beverage, milk beverage, diary beverage, plant protein drink, plant-based beverage, sport drink, energy drink.
47. The composition of any one of clauses 1-46, wherein the composition has a Brix value of about 1° to about 25°, or about 5° to about 20°, or about 7° to about 15°.
48. The composition of any one of clauses 1-47, wherein the composition has a total polyphenol content from about 0.002 wt % to about 2 wt %, or from about 0.01 wt % to about 1 wt %, or from about 0.1 wt % to about 0.5%.
49. The composition of any one of clauses 1-48, wherein the composition has a dosage of total polyphenol from 15 mg to about 300 mg, or from about 30 mg to about 200 mg, or from about 45 mg to about 150 mg for one serving.
50. The composition of any one of clauses 1-49, wherein the weight ratio of the total polyphenol to the total essential oil(s) is from about 1:10 to about 100:1, or from about 1:9 to about 90:1, or from about 1:8 to about 80:1, from about 1:7 to about 70:1, from about 1:6 to about 60:1, from about 1:5 to about 50:1, or from about 1:4 to about 40:1, or from about 1:3 to about 30:1, or from about 1:2 to about 20:1.
51. The composition of any one of clauses 1-50, wherein the composition has a total essential oil(s) content from about 0.001 wt. % to about 1 wt. %, or from about 0.05 wt. % to about 0.5 wt. %, or from about 0.1 wt. % to about 0.25%.
52. the composition of any one of clauses 1-51, wherein the composition has a dosage of the at least one essential oil(s) from 3 mg to about 150 mg, or from about 10 mg to about 100 mg, or from about 25 mg to about 50 mg for one serving.
53. The composition of any one of clauses 1-52, wherein the first polyphenol and the at least one essential oil(s) is derived from sage extract and wherein the dosage of sage extract is from about 50 mg to about 1,000 mg, or from about 100 mg to about 800 mg, or from about 200 mg to about 600 mg for one serving.
54 The composition of any one of clauses 1-22 or 24-39 or 41-53, wherein the first polyphenol is derived from sage and wherein the composition has a weight ratio of the first polyphenol derived from sage to the total essential oil(s) from about 2:1 to about 200:1.
55. The composition of any one of clauses 1-22 or 24-39 or 41-55, and wherein the first polyphenol is derived from sage and comprises at least one of: luteolin glycoside, luteolin glucuronide, and rosmarinic acid.
56. The composition of any one of clauses 8-32, wherein the first polyphenol is derived from grape seed extract and wherein the composition has a dosage of grape seed extract from about 50 mg to about 1,000 mg, or from about 100 mg to about 800 mg, or from about 200 mg to about 600 mg for one serving.
57. The composition of any one of clauses 8-33, wherein the first polyphenol is derived from grape seed and wherein the composition has a weight ratio the first polyphenol to the total essential oil(s) from about 2:1 to about 200:1.
58. The composition of any one of clauses 8-34, wherein the first polyphenol is derived from grape seed, and wherein the first polyphenol comprises at least one of: gallic acid, catechin, epicatechin, gallocatechin, epigallocatechin, and epicatechin 3-O-gallate, and procyanidin dimers, trimers, and more highly polymerized procyanidins.
59. A beverage comprising:
   a first polyphenol; and
   at least one essential oil(s);
   wherein the beverage has a weight ratio of total polyphenol to total essential oil(s) from about 1:10 to about 100:1; and
   wherein the first polyphenol is derived from sage.
60. The beverage of clause 59, wherein the at least one essential oil(s), comprises:
   terpenes, monoterpenes, sesquiterpenes, diterpenes, esters, aldehydes, ketones, alcohols, phenols and oxides, and derivatives and/or any combinations thereof.
61. The beverage of any one of clauses 59-60, wherein the first polyphenol and the at least one essential oil(s) are derived from the same plant species.
62. The composition of any one of clauses 1-2 or any one of clauses 59-60, wherein the first polyphenol and the at least one essential oils(s) are derived from different plants species.
63. The composition of any one of clauses 1-3, or any one of clauses 59-61, wherein the at least one essential oil(s) comprises,
   a first essential oil; and
   a second essential oil;
   wherein the first essential oil is derived from the same or different plant species as the first polyphenol, and the second essential oil is derived from a different plant species than the first essential oil.
64. The composition of any one of clauses 59-63, wherein the first polyphenol is derived from Salvia officinalis and wherein the at least one essential oil(s) is derived from Salvia lavandulifolia.
65. The beverage of any one of clauses 59-63, wherein the at least one essential oil(s) are derived from a plant genus selected from Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Origanum, Ocimum, Petroselimam, Pimpinella, Piper, Thymus, Zingiber, Humulus, and/or any combinations thereof 66. The beverage of clauses 59-65, further comprising a second polyphenol not derived from sage.
67. The beverage of clauses 66, wherein the second polyphenol is from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof.
68. The beverage of any one of clauses 59-67, wherein the beverage has a dosage of total polyphenol from 15 mg to about 300 mg, or from about 30 mg to about 200 mg, or from about 45 mg to about 150 mg for one serving.
69. The beverage of any one of clauses 59-68, wherein the weight ratio of the total polyphenol to the total essential oil(s) is from about 1:10 to about 100:1, or from about 1:50 to about 50:1, or from about 1:40 to about 40:1, from about 1:20 to about 20:1, from about 1:10 to about 10:1, from about 1:5 to about 5:1, or from about 1:3 to about 3:1, or from about 1:2 to about 2:1.
70. The beverage of any one of clauses 59-69, wherein the beverage has a dosage of the at least one essential oil(s) from 3 mg to about 150 mg, or from about 10 mg to about 100 mg, or from about 25 mg to about 50 mg for one serving.
71. A beverage comprising:
   a first polyphenol; and
   at least one essential oil(s),
   wherein the beverage has a weight ratio of total polyphenol to total essential oil(s) from about 1:10 to about 100:1;
   wherein the first polyphenol is derived from grape seed.
72. The beverage of clause 71, wherein the at least one essential oil(s), comprises:
   terpenes, monoterpenes, sesquiterpenes, diterpenes, esters, aldehydes, ketones, alcohols, phenols and oxides, and derivatives and/or any combinations thereof.
73. The beverage of any one of clauses 71 or 72, wherein the first polyphenol and the at least one essential oil(s) are derived from the same plant species.
74. The beverage of any one of clauses 71-73, wherein the first polyphenol and the at least one essential oils(s) are derived from different plants species.
75. The composition of any one of clauses 1-3, or any one of clauses 71-73,
   wherein the at least one essential oil(s) comprises,
   a first essential oil; and
   a second essential oil;
   wherein the first essential oil is derived from the same or different plant species as the first polyphenol, and the second essential oil is derived from a different plant species than the first essential oil.
76. The beverage of any one of clauses 71-75, wherein the at least one essential oil(s) are derived from a plant genus selected from Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schimis, Mentha, Origanum, Ocimum, Petroselimam, Pimpinella, Piper, Thymus, Zingiber, Humulus, and/or any combinations thereof
77. The beverage of clause 71, further comprising a second polyphenol not derived from grape seed.
78. The beverage of clause 77, wherein the second polyphenol is from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof.
79. The composition or beverage of any of the clauses 1-78, wherein administration of the composition or beverage to a human causes one or more effects on the human: improving cognitive function; improving mood; improving alertness; improving contentedness; improving happiness; improving friendliness; improving sociability; improving relaxation, increasing euphoria; improving conviviality; improving calmness; improving tranquility; improving mental capability; improving memory accuracy; improving speed of memory; improving relaxation; improving/sustaining attention; improving accuracy of attention; improving stroop effect, improving speed of attention; reducing stress; reducing tension; reducing mental fatigue; reducing anxiety; reducing inertia; reducing headache; promoting maintenance of normal sleep, reducing sleep onset latency, improving sleep quality, alleviating of the subjective feeling of jet leg, or any combinations thereof.
80. The composition or beverage of clause 79, wherein the composition or beverage takes effect after a time period following the administration, wherein the time period is about 5 minutes, or about 15 minutes, or about 30 minutes, or about 60 minutes, or about 90 minutes, or about 120 minutes or about 150 minutes, or about 200 minutes, or about 250 minutes, or about 300 minutes.
81. The composition or beverage of any one clauses 79-80, wherein the composition or beverage administered to the human comprises at least 15 mg, or at least 50 mg, or at least 75 mg, at least 100 mg, or at least 150 mg, or at least 200 mg, or at least 250 mg or at least 300 mg of total polyphenol.
82. The composition or beverage of any one of clauses 79-81, wherein the composition or beverage administered to the human comprises at least 3 mg, at least 50 mg, at least 100 mg, or at least 150 mg of total essential oil(s).
83. A method for improving a condition of a human, the method comprising:
   administering to the human in need of an improved condition an edible composition, wherein the composition comprises:
   a first polyphenol; and
   at least one essential oil(s),
wherein a ratio of total polyphenol to the total essential oil(s) is from about 1:10 to about 100:1.
84. The method of clause 83, wherein the at least one essential oil(s), comprises:
   terpenes, monoterpenes, sesquiterpenes, diterpenes, esters, aldehydes, ketones, alcohols, phenols and oxides, and derivatives and/or any combinations thereof.
85. The method of any one of clause 83-84, wherein the first polyphenol and the at least one essential oil(s) are derived from the same plant species.
86. The method of any one of clause 83-84, wherein the first polyphenol and the at least one essential oils(s) are derived from different plants species.
87. The method of any one of claims 83-86, wherein the at least one essential oil(s) comprises,
   a first essential oil; and
   a second essential oil;
   wherein the first essential oil is derived from the same or different plant species as the first polyphenol, and the second essential oil is derived from a different plant species than the first essential oil.
88. The method of any one of clauses 83-87, wherein the at least one essential oil(s) are derived from a plant genus selected from Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Origanum, Ocimum, Petroselinum, Pimpinella, Piper, Thymus, Zingiber, Humulus, and/or any combinations thereof.
89. The method of any one of clauses 83-88, wherein the first polyphenol is derived from a plant genus selected from Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasmimom, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.
90. The method of any one of clauses 83-89, wherein the first polyphenol is selected from a group comprising a flavonoid, a phenolic acid, a curcuminoid, a lignan, a stilbene, a phenolic terpene, a glycoside, a glucuronide, a depside and/or any combination thereof.
91. The method of clause 90, wherein the flavonoid is selected from a group comprising a flavanol, a flavone, a flavonol, a flavanone, an isoflavone, an anthocyanidin, a gallate, a glycoside, a polymer thereof and any combination thereof.
92. The composition of clause 91, wherein the flavanol is selected from a group comprising a catechin, a epicatechin, a catechin gallate, an epicatechin gallate, a gallocatechin, an epigallocatechin, a gallocatechin gallate, an epigallocatechin gallate, a proanthocyanidin and any combinations thereof.
93. The method of any one of clauses 91 or 92, wherein the flavone is selected from a group comprising a luteolin, an apigenin, a tangeritin, a chrysin, and any combination thereof.
94. The method of any one of clauses 91-93, wherein the flavonol is selected from a group comprising a quercetin, a kaempferol, a myricetin, a rutin, and any combination thereof.
95. The method of any one of clauses 91-94, wherein the flavanone is selected from a group comprising a eriodictyol, a hesperetin, a narigenin, and any combination thereof.
96. The method of any one of clauses 91-95, wherein the isoflavone is selected from a group comprising a genistein, a daidzein and any combination thereof.
97. The method of any one of clauses 91-96, wherein the anthocyanidin is selected from a group comprising a cyanidin, a delphinidin, a malvidin, a pelargonidin, an aaurantinidin, a capensinidin, an europinidin, a hirsutidin, a peonidin, a petunidin, a pulchellidin, a rosinidin and any combination thereof.
98. The method of clause 90, wherein the phenolic acid is selected from a group comprising a rosmarinic acid, a vanillic acid, a caffeic acid, a gallic acid, a protocatechuic acid, a salicyclic acid, a ferulic acid, a sinapic acid, a chlorogenic acids, a coumaric acid and any combination thereof.
99. The method of clause 90, wherein the curcuminoid is selected from a group comprising a bisdemethoxycurcumin, a demethoxycurcumin, a curcumin, and any combination thereof.
100. The method of clause 90, wherein the lignan is selected from a group comprising a lariciresinol, a pinoresino, a matairesinol, a syrigaresinol, a sesamin, a sesaminol, and any combination thereof.
101. The method of clause 90, wherein the stilbene is selected from a group comprising a resveratrol, a pterostilbene, and any combination thereof.
102. The method of clause 90, wherein the phenolic terpene is selected from a group comprising a carnosic acid, a rosmanol, a carnosol, and any combination thereof.
103. The method of any one of clauses 83-101, wherein the first polyphenol is derived from Salvia officinalis and the at least one essential oil(s) is derived from Salvia lavandulifolia.
104. The method of any one of clauses 83-103, wherein the first polyphenol is derived from grape seed.
105. The method of any one of clauses 83-104, further comprising a second polyphenol.
106. The method of clause 105, wherein the second polyphenol is selected from a group comprising a flavonoid, a phenolic acid, a curcuminoid, a lignan, a stilbene, a phenolic terpene, a glycoside, a glucuronide, a depside and/or any combination thereof.
107. The method of clause 106, wherein the flavonoid is selected from a group comprising a flavanol, a flavone, a flavonol, a flavanone, an isoflavone, an anthocyanidin, a gallate, a glycoside, a polymer thereof and any combination thereof.
108. The method of clause 107, wherein the flavanol is selected from a group comprising a catechin, an epicatechin, a catechin gallate, an epicatechin gallate, a gallocatechin, an epigallocatechin, a gallocatechin gallate, an epigallocatechin gallate, a proanthocyanidin and any combinations thereof.
109. The method of any one of clauses 107 or 108, wherein the flavone is selected from a group comprising a luteolin, an apigenin, a tangeritin, a chrysin, and any combination thereof.
110. The method of any one of clauses 107-109, wherein the flavonol is selected from a group comprising a quercetin, a kaempferol, a myricetin, a rutin, and any combination thereof.
111. The method of any one of clauses 107-110, wherein the flavanone is selected from a group comprising a eriodictyol, a hesperetin, a narigenin, and any combination thereof.
112. The method of any one of clauses 107-111, wherein the isoflavone is selected from a group comprising a genistein, a daidzein and any combination thereof.
113. The method of any one of clauses 107-112, wherein the anthocyanidin is selected from a group comprising a cyanidin, a delphinidin, a malvidin, a pelargonidin, an aaurantinidin, a capensinidin, an europinidin, a hirsutidin, a peonidin, a petunidin, a pulchellidin, a rosinidin and any combination thereof.
114. The method of clause 106, wherein the phenolic acid is selected from a group comprising a rosmarinic acid, a vanillic acid, a caffeic acid, a gallic acid, a protocatechuic acid, a salicyclic acid, a ferulic acid, a sinapic acid, a chlorogenic acids, a coumaric acid and any combination thereof.
115. The method of clause 106, wherein the curcuminoid is selected from a group comprising a bisdemethoxycurcumin, a demethoxycurcumin, a curcumin, and any combination thereof.
116. The method of clause 106, wherein the lignan is selected from a group comprising a lariciresinol, a pinoresino, a matairesinol, a syrigaresinol, a sesamin, a sesaminol, and any combination thereof.
117. The method of clause 106, wherein the stilbene is selected from a group comprising a resveratrol, a pterostilbene, and any combination thereof.
118. The method of clause 106, wherein the phenolic terpene is selected from a group comprising a carnosic acid, a rosmanol, a carnosol, and any combination thereof.
119. The method of any one of clauses 83-118, wherein the second polyphenol is derived from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof.
120. The method of any one of clauses 83-119, wherein the second polyphenol is derived from a plant genus selected from Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasming m, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.
121. The method of any one of clauses 83-120, wherein the second polyphenol is not derived from sage or grape seed.
122. The method of any one of clauses 83-121, further comprising a sweetener.
123. The method of clause 122, wherein the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs), and combinations thereof.
124. The method of any one of clauses 83-123, further comprising at least one additive or functional ingredient or both.
125. The method of any one of clauses 83-124, wherein the composition is in a drinkable liquid form, a concentrate form, a dry form, or a semi-dry form. 126 The method of any one of clauses 83-124, wherein the composition is a gum, gel, tablet, capsule, granule, cubic, or dry powder.
127. The method of any one of clauses 83-125, wherein the composition is a beverage selected from the group of non-carbonated beverage, carbonated beverage, juice beverage, fruit juice, coffee beverage, tea beverage, milk beverage, diary beverage, plant protein drink, plant-based beverage, sport drink, energy drink.
128. The method of any one of clauses 83-127, wherein the composition has a Brix value of about 1° to about 25°, or about 5° to about 20°, or about 7° to about 15°.
129. The method of any one of clauses 83-128, wherein the composition has a total polyphenol content from about 0.002 wt % to about 2 wt %, or from about 0.01 wt % to about 1 wt %, or from about 0.1 wt % to about 0.5%.
130. The method of any one of clauses 83-129, wherein the composition has a dosage of total polyphenol from 15 mg to about 300 mg, or from about 30 mg to about 200 mg, or from about 45 mg to about 150 mg for one serving.
131. The method of any one of clauses 83-130, wherein the weight ratio of the total polyphenol to the total essential oil(s) is from about 1:10 to about 100:1, or from about 1:9 to about 90:1, or from about 1:8 to about 80:1, from about 1:7 to about 70:1, from about 1:6 to about 60:1, from about 1:5 to about 50:1, or from about 1:4 to about 40:1, or from about 1:3 to about 30:1, or from about 1:2 to about 20:1.
132. The method of any one of clauses 83-131, wherein the composition has a total essential oil(s) content from about 0.001 wt. % to about 1 wt. %, or from about 0.05 wt. % to about 0.5 wt. %, or from about 0.1 wt. % to about 0.25%.
133. The method of any one of clauses 83-132, wherein the composition has a dosage of the at least one essential oil(s) from 3 mg to about 150 mg, or from about 10 mg to about 100 mg, or from about 25 mg to about 50 mg for one serving.
134 The method of any one of clauses 83-133, wherein the first polyphenol and the at least one essential oil(s) is derived from sage extract and wherein the dosage of the sage extract is from about 50 mg to about 1,000 mg, or from about 100 mg to about 800 mg, or from about 200 mg to about 600 mg for one serving.
135. The method of any one of clauses 83-103 or 105-120 or 122-134, wherein the first polyphenol is derived from sage and wherein the composition has a weight ratio of the first polyphenol derived from sage to the total essential oil(s) from about 2:1 to about 200:1.
136. The method of any one of clauses 83-103 or 105-120 or 122-135, and wherein the first polyphenol is derived from sage and comprises at least one of: luteolin glycoside, luteolin glucuronide, and rosmarinic acid.
137 The composition of any one of clauses 83-102 or 104-133, wherein the first polyphenol is derived from grape seed extract and wherein the composition has a dosage grape seed extract from about 50 mg to about 1,000 mg, or from about 100 mg to about 800 mg, or from about 200 mg to about 600 mg for one serving.
138. The composition of any one of clauses 83-102, 104-133 or 136, wherein the first polyphenol is derived from grape seed and wherein the composition has a weight ratio the first polyphenol to the total essential oil(s) from about 2:1 to about 200:1.
139 The composition of any one of clauses 83-102, 104-133 or 136-138, wherein the first polyphenol is derived from grape seed, and wherein the first polyphenol comprises at least one of: gallic acid, catechin, epicatechin, gallocatechin, epigallocatechin, and epicatechin 3-O-gallate, and procyanidin dimers, trimers, and more highly polymerized procyanidins.
140. A method for improving a condition of a human, the method comprising:
   administering to the human in need of an improved condition a beverage comprising:
   a first polyphenol; and
   at least one essential oil(s),
   wherein the beverage has a weight ratio of the total polyphenol to total essential oil(s) from about 1:10 to about 100:1, and wherein the first polyphenol is derived from sage.
141. The method of clause 140, wherein the at least one essential oil(s) comprises,
   a first essential oil; and
   a second essential oil;
   wherein the first essential oil is derived from the same or different plant species as the first polyphenol, and the second essential oil is derived from a different plant species than the first essential oil.
142. The method of clauses 140-141, wherein the at least one essential oil(s) comprises a first essential oil derived from sage and at least a second essential oil not derived from sage.
143. The method of any one of clauses 140-142, further comprising a second polyphenol not derived from sage.
144. The method of any one of clauses 140-143, wherein the second polyphenol is from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof.
145. A method for improving a condition of a human, the method comprising:
   administering to the human in need of an improved condition a beverage comprising:
      a first polyphenol; and
      at least one essential oil(s);
   wherein the beverage has a weight ratio of the total polyphenol to the total essential oil(s) from about 1:10 to about 100:1, wherein the first polyphenol is derived from grape seed.
146. The method of clause 145, wherein the at least one essential oil(s) comprises,
   a first essential oil; and
   a second essential oil;
   wherein the first essential oil is derived from the same or different plant species as the first polyphenol, and the second essential oil is derived from a different plant species than the first essential oil.
147. The method of any one of clauses 145-146, further comprising a second polyphenol not derived from grape seed.
148. The method of any one of clauses 145-147, wherein the second polyphenol is from a natural resource, wherein the natural resource is a plant selected from the group of coffee, tea, yerba mate, guayusa, yaupon, guarana, cocoa, kola, berries, non-berry fruits, vegetables, seasonings, beans, nuts, soy, or any combination thereof.
149. The method of any one of clauses 145-148, wherein administration of the composition or beverage to the human causes one or more effects on the human: improving cognitive function; improving mood; improving happiness; improving friendliness; improving sociability; improving relaxation, increasing euphoria; improving conviviality; improving alertness; improving contentedness; improving calmness; improving tranquility; improving mental capability; improving memory accuracy; improving speed of memory; improving relaxation; improving/sustaining attention; improving accuracy of attention; improving stroop effect, improving speed of attention; reducing stress; reducing tension; reducing mental fatigue; reducing anxiety; reducing inertia; reducing headache; promoting maintenance of normal sleep, reducing sleep onset latency, improving sleep quality, alleviating of the subjective feeling of jet leg, or any combinations thereof.
150. The method of any one of clauses 145-149, wherein the composition or beverage takes effect after a time period following the administration, wherein the time period is about 5 minutes, or about 15 minutes, or about 30 minutes, or about 60 minutes, or about 90 minutes, or about 120 minutes or about 150 minutes, or about 200 minutes, or about 250 minutes, or about 300 minutes.
151. The method of any one of clauses 145-150, wherein the composition or beverage administered to the human comprises at least 15 mg, or at least 50 mg, or at least 75 mg, at least 100 mg, or at least 150 mg, or at least 200 mg, or at least 250 mg or at least 300 mg of total polyphenol.
152. The method of any one of clauses 145-151, wherein the composition or beverage administered to the human comprises at least 3 mg, at least 50 mg, at least 100 mg, or at least 150 mg of total essential oil(s).
153. An orally consumable composition comprising:
   (i) at least one aqueous extractive(s) from a plant; and/or
   (ii) at least one essential oil(s) from the same or different plant; and/or
   (iii) caffeine, wherein the caffeine is from a source other than (i) and/or (ii);
   wherein (i) and/or (ii) form a plant extract composition; and wherein the ratio of total caffeine to plant extract composition is from 1:200 to 40:1 by weight (w/w).
154. The orally consumable composition of clause 153, wherein the at least one essential oil(s), comprise terpenes, monoterpenes, sesquiterpenes, diterpenes, esters, aldehydes, ketones, alcohols, phenols and oxides, and derivatives and/or combinations thereof.
155. The orally consumable composition of any one of clauses 153-154, wherein if both the essential oil(s) and the at least one aqueous extractive(s) are present, the ratio of the at least one essential oil(s) to the at least one aqueous extractive(s) is from 1:10 to 1:800.
156. The orally consumable composition of any one of clauses 153-155, wherein the at least one essential oils(s) comprise plant essential oil derived from the plant root, rhizomes, stem, bark, twig, leaves, flowers, fruits, seeds and combinations thereof.
157. The orally consumable composition of any one of clauses 153-156, wherein the plant extract composition is derived from a plant genus selected from a group comprising Rosa, Sophora, Citrus, Fragaria, Juniperus, Murraya, Sambucus, Syzygium, Angelica, Hibiscus, Helichrysum, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselinum, Pimpinella, Piper, Thymus, Zingiber, Humulus, Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Juglans, Litchi, Lycium, Malus, Mangifera, Olea, Passiflora, Prunus, Punica, Vaccinium, Vitis, Aronia, Aloe, Bacopa, Daucus, Euterpe, Malpighia, Morus, Phyllanthus, Rubus, Satureja and combinations thereof.
158. The orally consumable composition of any one of clauses 153-157, wherein the plant extract composition comprises Salvia and Camellia species.
159. The orally consumable composition of any one of clauses 153-158, wherein the plant extract composition comprises a Salvia species, wherein the Salvia species is selected from S. officinalis, S. lavandulifolia, S. rosmarinis (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.
160. The orally consumable composition of any one of clauses 153-159, wherein the plant extract composition comprises a Eucalyptus species, wherein the Eucalyptus species is selected from a group comprising Eucalyptus species is selected from E. globulus, E. cneorifolia, E. dives, E. Dumosa, E. goniocalyx, E. horistes, E. kochii, E. leucoxylon, E. largiflorens, E. oleosa, E. polybractea, E. radiata, E. rossii, E. sideroxylon, E. smithii, E. staigeriana, E. tereticornis, E. viridis and combinations thereof.
161. The orally consumable composition of any one of clauses 153-160, wherein the plant extract composition comprises a Mentha species, wherein the Mentha species is selected from a group comprising Mentha piperita, M. spicata, M. citrate and combinations thereof.
162. The orally consumable composition of any one of clauses 153-161, wherein the plant extract composition comprises a Thymus species, wherein the Thymus species is selected from a group comprising Thymus vulgaris, T. zygis, T. serpyllum, T. citriodorus, T. praecox, T. pseudolanuginosus and combinations thereof.
163. The orally consumable composition of any one of clauses 153-162, wherein the plant extract composition comprises a Ocimum species, wherein the Ocimum species is selected from a group comprising Ocimum basilicum, O. citriodorum, O. kilimandscharicum, O. Americanum, O. gratissimum, O. tenuiflorum and combinations thereof.
164. The orally consumable composition of any one of clauses 153-163, wherein the plant extract composition comprises a Camellia species, wherein the Camellia species is selected from the group comprising Camellia sinensis, for examples C. sinensis var. sinensis and C. sinensis var. assamica.
165. The orally consumable composition of any one of clauses 153-164, wherein the plant extract composition comprises a Camellia species and a Salvia species, wherein the Camellia species are selected from a group consisting of Camellia sinensis, for examples C. sinensis var. sinensis and C. sinensis var assamica and wherein the Salvia species are selected from a group consisting of S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.
166. The orally consumable composition of any one of clauses 153-165, wherein the composition comprises Camellia species and Salvia species.
167. The orally consumable composition of any one of clauses 153-166, wherein the composition comprises a powder, concentrate, or infusion, or a combination thereof of a Camellia species and/or Salvia species, or wherein the aqueous extractives comprises a powder, a concentrate, an infusion, or a combination thereof of a Camellia species and/or Salvia species.
168. The orally consumable composition of any one of clauses 153-167, wherein the composition comprises a Camellia species in the form of a green tea, a white tea, a yellow tea, a black tea, a dark tea, or an oolong tea.
169. The orally consumable composition of any one of clauses 153-168, wherein the composition comprises:
   (i) between 100 mg to 4.0 g of aqueous extractive of Camellia species, in the form of a powder, a concentrate, or a concentrated infusion; and
   (iii) between 100 mg and 500 mg of aqueous extractive of Salvia species.
170. An orally consumable composition comprising:
   (i) caffeine, wherein a total caffeine content is less than 100 mg;
   (ii) a plant extract composition comprising at least one aqueous extractive(s) from a plant and/or at least one essential oil(s) from the same or different plant, wherein the plant extract composition is between 50 mg to 3000 mg; and
   wherein the plant extract composition is derived from plant genus selected from one or more of the following: Rosa, Sophora, Citrus, Fragaria, Juniperus, Murraya, Sambucus, Syzygium, Angelica, Hibiscus, Helichrysum, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselimam, Pimpinella, Piper, Thymus, Zingiber, Humulus, Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Juglans, Litchi, Lycium, Malus, Mangifera, Olea, Passiflora, Prunus, Punica, Vaccinium, Vitis, Aronia, Aloe, Bacopa, Daucus, Euterpe, Malpighia, Morus, Phyllanthus, Rubus, Satureja and combinations thereof.
171. The orally consumable composition of clause 170, comprising one or more of Salvia and Camellia.
172. The orally consumable composition of clause 170, comprising Coffea.
173. The orally consumable composition of clause 170, comprising Camellia.
174. The orally consumable composition of clause 170, comprising Salvia.
175. The orally consumable composition of clause 170, comprising from 1 mg to 25 mg of the at least one essential oil(s).
176. The orally consumable composition of clauses 170-175, wherein the consumable composition comprises: a rosmarinic acid and/or derivatives thereof in an amount of from 2.5 mg to 100 mg;
   a carnosol, a carnosic acid, and/or derivatives thereof in an amount of from 0.5 mg to 100 mg;
   caffeic acid and/or derivatives thereof in an amount of from 2.5 mg to 100 mg; and
   luteolin and/or derivatives thereof in an amount of from 2.5 mg to 100 mg, in a total amount of up to 500 mg.
177. The orally consumable composition of clauses 170-176, wherein the composition further comprises total catechin in an amount of about 25 mg to about 1000 mg.
178. The orally consumable composition of clauses 170-177, wherein the composition further comprises total catechin in an amount of about 50 mg to about 600 mg.
179. The orally consumable composition of clauses 170-178, wherein the consumable composition comprises:
   a rosmarinic acid and/or derivatives thereof in an amount of from 2.5 mg to 100 mg;
   a carnosol, a carnosic acid, and/or derivatives thereof in an amount of from 0.5 mg to 100 mg;
   caffeic acid and/or derivatives thereof in an amount of from 2.5 mg to 100 mg;
luteolin and/or derivatives thereof in an amount of from 2.5 mg to 100 mg, in a total amount of up to 500 mg; and
   total catechin in an amount of about 25 mg to about 1000 mg.
180. The orally consumable composition of any one of clauses 170-179, wherein the plant extract composition is a S. officinalis extract composition that comprises rosmarinic acid in an amount of about 3.5 mg to 50 mg.
81. The orally consumable composition of any one of claims 170-180, wherein the plant extract composition comprises a Camellia species, wherein the Camellia species is selected from the group comprising Camellia sinensis, for examples C. sinensis var. sinensis and C. sinensis var. assamica.
182. The orally consumable composition of any one of claims 170-181, wherein the plant extract composition comprises a Camellia species and a Salvia species, wherein the Camellia species are selected from a group consisting of Camellia sinensis, for examples C. sinensis var. sinensis and C. sinensis var. assamica and wherein the Salvia species are selected from a group consisting of S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.
183. The orally consumable composition of any one of clauses 170-182, comprising about 100 mg to about 400 mg of the plant extract composition, wherein the plant extract composition is a S. officinalis extract composition that comprises rosmarinic acid in an amount of about 3.5 mg to about 50 mg, and total polyphenol in an amount of about 20 mg to about 150 mg.
184. An orally consumable composition of any one of clauses 170-183, comprising about from about 200 mg to about 600 mg, from about 400 mg to about 700 mg, from about 500 mg to about 600 mg, or about 312 mg of a flavanol, the flavanol being total catechin; from about 10 mg to about 200 mg, from about 30 mg to about 80 mg, from about 100 mg to about 200 mg, or about 62 mg of caffeine; and from about 5 mg to about 80 mg, from about 10 mg to about 60 mg, from about 30 mg to about 50 mg, or about 23.8 mg of total amino acid.
185. An orally consumable composition of any one of clauses 170-184 comprising from about 10 mg to about 200 mg, from about 30 mg to about 100 mg, from about 50 mg to about 80 mg, or about 70 mg of a flavanol, the flavanol being catechin; about 5 mg to about 75 mg, about 10 mg to about 50 mg, about 15 mg to about 30 mg, or about 15.6 mg of caffeine; and about 5 mg to about 80 mg, about 10 mg to about 50 mg, about 15 mg to about 30 mg, or about 13.7 mg of total amino acid.
186. An orally consumable composition of any one of clauses 170-185, comprising:
   (i) from about 200 mg to about 600 mg, from about 400 mg to about 700 mg, from about 500 mg to about 600 mg, or about 312 mg of a flavanol, the flavanol being catechin; from about 10 mg to about 200 mg, from about 30 mg to about 80 mg, from about 100 mg to about 200 mg, or about 62 mg of caffeine; and from about 5 mg to about 80 mg, from about 10 mg to about 60 mg, from about 30 mg to about 50 mg, or about 23.8 mg of total amino acid.
   (ii) from about 10 mg to about 200 mg, from about 30 mg to about 100 mg, from about 50 mg to about 80 mg, or about 70 mg of a flavanol, the flavanol being catechin; about 5 mg to about 75 mg, about 10 mg to about 50 mg, about 15 mg to about 30 mg, or about 15.6 mg of caffeine; and about 5 mg to about 80 mg, about 10 mg to about 50 mg, about 15 mg to about 30 mg, or about 13.7 mg of total amino acid.
   (iii) about 3.5 mg to 50 mg of a plant extract, the plant extract composition being a S. officinalis extract composition that comprises rosmarinic acid;
   wherein (i) and (ii) comprise a Camellia species and (iii) comprises a Salvia species, wherein the Camellia species are selected from a group consisting of Camellia sinensis, for examples C. sinensis var. sinensis and C. sinensis var. assamica and wherein the Salvia species are selected from a group consisting of S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.
187. The orally consumable composition of any one of clauses 170-186, wherein the orally consumable composition comprises about less than 75 mg caffeine but greater than zero mg of caffeine.
188. A method for providing a cognitive benefit to a human subject, comprising orally administering to the subject an orally consumable composition according to any one of clauses 153-187.
189. The method of clause 188, wherein the cognitive benefit is selected from a group comprising improving cognitive function; improving mood; improving happiness; improving friendliness; improving sociability; improving relaxation, increasing euphoria; improving conviviality; improving alertness; improving contentedness; improving calmness; improving tranquility; improving mental capability; improving memory accuracy; improving speed of memory; improving relaxation; improving/sustaining attention; improving accuracy of attention; improving stroop effect, improving speed of attention; reducing stress; reducing tension; reducing mental fatigue; reducing anxiety, reducing inertia; reducing headache; promoting maintenance of normal sleep, reducing sleep onset latency, improving sleep quality, alleviating of the subjective feeling of jet leg, or any combinations thereof.
190. The method of any one of clauses 188-189, wherein the method further comprises the step of conducting a cognitive assessment of the subject prior to and/or after the subject has consumed the orally consumable composition of any one of the clauses 153-187.
191. An edible composition for improving a condition of a human, the composition comprising:
   a first polyphenol; and
   at least one essential oil(s),
   wherein a ratio of total polyphenol to total essential oil(s) is from about 1:10 to about 100:1.
192. The composition of clause 191, wherein the at least one essential oil(s), comprises:
   terpenes, monoterpenes, sesquiterpenes, diterpenes, esters, aldehydes, ketones, alcohols, phenols and oxides, and derivatives and/or any combinations thereof.
193. The composition of any one of clauses 191-192, wherein the at least one essential oil(s) comprises:
   a first essential oil; and
   a second essential oil;
   wherein the first essential oil is derived from the same or different plant species as the first polyphenol, and the second essential oil is derived from a different plant species than the first essential oil.
194. The composition of any one of clauses 191-193, wherein the at least one essential oil(s) are derived from a plant genus selected from Rosa, Sophora, Citrus, Fragaria, Juniperus, Murraya, Sambucus, Syzygium, Angelica, Hibiscus, Helichrysum, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselinum, Pimpinella, Piper, Thymus, Zingiber, and/or any combinations thereof.
195. The composition of any one of clauses 191-194, wherein the first polyphenol is derived from Salvia officinalis and wherein the at least one essential oil(s) is derived from Salvia lavandulifolia.
196. The composition of any one of clauses 191-195, wherein the first polyphenol is derived from grape seed extract and wherein the composition has a dosage of grape seed extract from about 50 mg to about 1,000 mg, or from about 100 mg to about 800 mg, or from about 200 mg to about 600 mg for one serving.
197. The composition of any one of clauses 191-196, wherein the composition has a total polyphenol content from about 0.002 wt % to about 2 wt %, or from about 0.01 wt % to about 1 wt %, or from about 0.1 wt % to about 0.5%.
198. A beverage comprising:
   a first polyphenol; and
   at least one essential oil(s);
   wherein the beverage has a weight ratio of total polyphenol to total essential oil(s) from about 1:10 to about 100:1; and
   wherein the first polyphenol is derived from sage.
199. The beverage of clause 198, wherein the at least one essential oil(s), comprises:
   terpenes, monoterpenes, sesquiterpenes, diterpenes, esters, aldehydes, ketones, alcohols, phenols and oxides, and derivatives and/or any combinations thereof.
200. The beverage of any one of clauses 198-199, wherein the beverage has a dosage of total polyphenol from 15 mg to about 300 mg, or from about 30 mg to about 200 mg, or from about 45 mg to about 150 mg for one serving.
201. The beverage of any one of clauses 198-200, wherein the beverage has a dosage of the at least one essential oil(s) from 3 mg to about 150 mg, or from about 10 mg to about 100 mg, or from about 25 mg to about 50 mg for one serving.
202. A method for improving a condition of a human, the method comprising:
   administering to the human in need of an improved condition an edible composition, wherein the composition comprises:
   a first polyphenol; and
   at least one essential oil(s);
   wherein a ratio of total polyphenol to the total essential oil(s) is from about 1:10 to about 100:1.
203. The method of clause 202, wherein the at least one essential oil(s), comprises:
   terpenes, monoterpenes, sesquiterpenes, diterpenes, esters, aldehydes, ketones, alcohols, phenols and oxides, and derivatives and/or any combinations thereof.
204. The method of any one of clauses 202-203, wherein the at least one essential oil(s) are derived from a plant genus selected from Rosa, Sophora, Citrus, Fragaria, Juniperus, Murraya, Sambucus, Syzygium, Angelica, Helichrysum, Hibiscus, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Origanum, Ocimum, Petroselinum, Pimpinella, Piper, Thymus, Zingiber, Humulus, and/or any combinations thereof.
205. The method of any one of clauses 202-204, wherein the first polyphenol is derived from a plant genus selected from Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Rosa, Sophora, Citrus, Fragaria, Juglans, Litchi, Lycium, Malus, Mangifera, Murraya, Olea, Passiflora, Prunus, Punica, Sambucus, Syzygium, Vaccinium, Vitis, Aronia, Angelica, Hibiscus, Chamomilla, Chamaemelum, Jasminum, Lavandula, Melissa, Valerian, Anthemis, Amomum, Cinnamomum, Coriandrum, Elettaria, Eucalyptus, Capsicum, Curcuma, Foeniculum, Salvia, Satureia, Schinus, Mentha, Ocimum, Petroselinum, Pimpinella, Piper, Thymus, Zingiber, Aloe, Bacopa, Daucus, Euterpe, Humulus, Juniperus, Malpighia, Morus, Phyllanthus, Rubus, Satureja.
206. The method of any one of clauses 202-205, wherein the first polyphenol is derived from grape seed.
207. The method of any one of clauses 202-206, further comprising a second polyphenol.
208. The method of any one of clauses 202-207, wherein the composition has a Brix value of about 1° to about 25°, or about 5° to about 20°, or about 7° to about 15°.
209. The method of any one of clauses 202-208, wherein the composition has a dosage of total polyphenol from 15 mg to about 300 mg, or from about 30 mg to about 200 mg, or from about 45 mg to about 150 mg for one serving.
210. The method of any one of clauses 202-209, wherein the composition has a total essential oil(s) content from about 0.001 wt. % to about 1 wt. %, or from about 0.05 wt. % to about 0.5 wt. %, or from about 0.1 wt. % to about 0.25%.

What is claimed is:

1. An orally consumable composition comprising:
(i) at least one aqueous extractive(s) from a plant; and/or
(ii) at least one essential oil(s) from the same or different plant; and/or
(iii) caffeine, wherein the caffeine is from a source other than (i) and/or (ii);
wherein (i) and/or (ii) form a plant extract composition; and wherein the ratio of total caffeine to total plant extract composition is from 1:200 to 40:1, or about 1:200 to about 1:1, or about 1:200 to about 1:20 by weight (w/w).

2. The orally consumable composition of claim 1, wherein the at least one essential oil(s), comprise terpenes, monoterpenes, sesquiterpenes, diterpenes, esters, aldehydes, ketones, alcohols, phenols and oxides, and derivatives and/or combinations thereof; and
wherein if both the essential oil(s) and the at least one aqueous extractive(s) are present, the ratio of the at least one essential oil(s) to the at least one aqueous extractive(s) is from 1:10 to 1:800, or about 1:1 to about 1:800, or about 1:100 to about 1:800, or about 1:200 to about 1:800.

3. The orally consumable composition of claim 2, wherein the at least one essential oils(s) comprise plant essential oil derived from the plant root, rhizomes, stem, bark, twig, leaves, flowers, fruits, seeds, and combinations thereof.

4. The orally consumable composition of any one of claims 1-3, wherein the plant extract composition is derived from a plant genus selected from a group comprising Rosa, Sophora, Citrus, Fragaria, Juniperus, Murraya, Sambucus, Syzygium, Angelica, Hibiscus, Helichrysum, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselinum, Pimpinella, Piper, Thymus, Zingiber, Humulus, Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Juglans, Litchi, Lycium, Malus, Mangifera, Olea, Passiflora, Prunus, Punica, Vaccinium, Vitis, Aronia, Aloe, Bacopa, Daucus, Euterpe, Malpighia, Morus, Phyllanthus, Rubus, Satureja and combinations thereof.

5. The orally consumable composition of any one of claims 1-4, wherein the plant extract composition comprises a Salvia species, wherein the Salvia species is selected from S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum; or
wherein the plant extract composition comprises a Eucalyptus species, wherein the Eucalyptus species is selected from a group comprising Eucalyptus species is selected from E. globulus, E. cneorifolia, E. dives, E. Dumosa, E. goniocalyx, E. horistes, E. kochii, E. leucoxylon, E. largiflorens, E. oleosa, E. polybractea, E. radiata, E. rossii, E. sideroxylon, E. smithii, E. staigeriana, E. tereticornis, E. viridis and combinations thereof; or
wherein the plant extract composition comprises a Mentha species, wherein the Mentha species is selected from a group comprising Mentha piperita, M. spicata, M. citrate and combinations thereof; or
wherein the plant extract composition comprises a Thymus species, wherein the Thymus species is selected from a group comprising Thymus vulgaris, T. zygis, T. serpyllum, T. citriodorus, T. praecox, T. pseudolanuginosus and combinations thereof; or
wherein the plant extract composition comprises a Ocimum species, wherein the Ocimum species is selected from a group comprising Ocimum basilicum, O. citriodorum, O. kilimandscharicum, O. Americanum, O. gratissimum, O. tenuiflorum and combinations thereof; or
wherein the plant extract composition comprises a Camellia species, wherein the Camellia species is selected from the group comprising Camellia sinensis, for examples C. sinensis var. sinensis and C. sinensis var. assamica, and combinations thereof; or
wherein the plant extract composition comprises a Camellia species and a Salvia species, wherein the Camellia species are selected from a group consisting of Camellia sinensis, for examples C. sinensis var. sinensis and C. sinensis var. assamica, and combinations thereof and wherein the Salvia species are selected from a group consisting of S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum, and combinations thereof.

6. The orally consumable composition of claim 2, wherein the plant extract composition comprises a powder, concentrate, or infusion of a Camellia species and/or Salvia species.

7. The orally consumable composition of claim 6, wherein the plant extract composition comprises a Camellia species in the form of a green tea, a white tea, a yellow tea, a black tea, a dark tea, or an oolong tea.

8. The orally consumable composition of claim 6, wherein the composition comprises:
(i) between 100 mg to 4.0 g of an aqueous extractive of Camellia species, in the form of a powder, a concentrate, or a concentrated infusion; and
(iii) between 100 mg and 500 mg of an aqueous extractive of Salvia species.

9. An orally consumable composition comprising:
(i) caffeine, wherein a total caffeine content is less than 100 mg;
(ii) a plant extract composition comprising at least one aqueous extractive(s) from a plant and/or at least one essential oil(s) from the same or different plant, wherein the plant extract composition is between 50 mg to 3000 mg; and
wherein the plant extract composition is derived from plant genus selected from one or more of the following: Rosa, Sophora, Citrus, Fragaria, Juniperus, Murraya, Sambucus, Syzygium, Angelica, Hibiscus, Helichrysum, Chamomilla, Chamaemelum, Cymbopogon, Geranium, Matricaria, Jasminum, Lavandula, Melissa, Valerian, Agave, Anthemis, Amomum, Cinnamomum, Coriandrum, Elettaria, Eucalyptus, Capsicum, Curcuma, Euonymus, Foeniculum, Litsea, Salvia, Satureia, Schinus, Mentha, Ocimum, Origanum, Petroselinum, Pimpinella, Piper, Thymus, Zingiber, Humulus, Camellia, Coffea, Ilex, Paullinia, Theobroma, Hypericum, Beta, Glycine, Ginkgo, Matricaria, Moringa, Magnolia, Withania, Aspalathus, Euterpe, Crocus, Polygonum, Ribes, Juglans, Litchi, Lycium, Malus, Mangifera, Olea, Passiflora, Prunus, Punica, Vaccinium, Vitis, Aronia, Aloe, Bacopa, Daucus, Euterpe, Malpighia, Morus, Phyllanthus, Rubus, Satureja and combinations thereof.

10. The orally consumable composition of claim 9, comprising one or more plant extract compositions of Salvia and/or Camelia plant genus.

11. The orally consumable composition of claim 9, wherein the plant extract composition comprises Coffea, or wherein the plant extract composition comprises Camelia, or wherein the plant extract composition comprises Salvia,

12. The orally consumable composition of claim 9, comprising from 1 mg to 25 mg of the at least one essential oil(s).

13. The orally consumable composition of claim 10, wherein the consumable composition comprises:
a rosmarinic acid and/or derivatives thereof in an amount of from 2.5 mg to 100 mg;
a carnosol, a carnosic acid, and/or derivatives thereof in an amount of from 0.5 mg to 100 mg;
caffeic acid and/or derivatives thereof in an amount of from 2.5 mg to 100 mg;
and
luteolin and/or derivatives thereof in an amount of from 2.5 mg to 100 mg, in a total amount of up to 500 mg.

14. The orally consumable composition of claim 9, wherein the composition further comprises total catechin in an amount of about 25 mg to about 1000 mg, or wherein the composition further comprises a total catechin in an amount of about 50 mg to about 600 mg.

15. The orally consumable composition of claim 10, wherein the consumable composition comprises:
a rosmarinic acid and/or derivatives thereof in an amount of from 2.5 mg to 100 mg;
a carnosol, a carnosic acid, and/or derivatives thereof in an amount of from 0.5 mg to 100 mg;
caffeic acid and/or derivatives thereof in an amount of from 2.5 mg to 100 mg;
luteolin and/or derivatives thereof in an amount of from 2.5 mg to 100 mg, in a total amount of up to 500 mg; and
total catechin in an amount of about 25 mg to about 1000 mg.

16. The orally consumable composition of claim 9, wherein the plant extract composition is a S. officinalis extract composition that comprises rosmarinic acid in an amount of about 3.5 mg to 50 mg.

17. The orally consumable composition of claim 9, wherein the plant extract composition comprises a Camellia species, wherein the Camellia species is selected from the group comprising Camellia sinensis, for examples C. sinensis var. sinensis and C. sinensis var. assamica, or
wherein the plant extract composition comprises a Camellia species and a Salvia species, wherein the Camellia species are selected from a group consisting of Camellia sinensis, for examples C. sinensis var. sinensis and C. sinensis var. assamica and wherein the Salvia species are selected from a group consisting of S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.

18. The orally consumable composition of claim 9, comprising about 100 mg to about 400 mg of the plant extract composition, wherein the plant extract composition is a S. officinalis extract composition that comprises rosmarinic acid in an amount of about 3.5 mg to about 50 mg, and total polyphenol in an amount of about 20 mg to about 150 mg.

19. An orally consumable composition of claim 9, comprising about from about 200 mg to about 700 mg, from about 400 mg to about 600 mg, from about 500 mg to about 600 mg, or about 312 mg of a flavanol, the flavanol being total catechin; from about 10 mg to about 200 mg, from about 30 mg to about 80 mg, from about 100 mg to about 200 mg, or about 62 mg of caffeine; and from about 5 mg to about 80 mg, from about 10 mg to about 60 mg, from about 30 mg to about 50 mg, or about 23.8 mg of total amino acid.

20. An orally consumable composition of claim 9, comprising from 10 mg to about 200 mg, about 30 mg to about 100 mg, about 50 mg to about 80 mg, or about 70 mg of a flavanol, the flavanol being catechin;
about 5 mg to about 75 mg, about 10 mg to about 50 mg, about 15 mg to about 30 mg, or about 15.6 mg of caffeine; and
about 5 mg to about 80 mg, about 10 mg to about 50 mg, about 15 mg to about 30 mg, or about 13.7 mg of total amino acid.

21. An orally consumable composition of claim 9, comprising:
(i) from about 200 mg to about 600 mg, from about 400 mg to about 700 mg, from about 500 mg to about 600 mg, or about 312 mg of a flavanol, the flavanol being catechin; from about 10 mg to about 200 mg, from about 30 mg to about 80 mg, from about 100 mg to about 200 mg, or about 62 mg of caffeine; and from about 5 mg to about 80 mg, from about 10 mg to about 60 mg, from about 30 mg to about 50 mg, or about 23.8 mg of total amino acid;
(ii) from about 10 mg to about 200 mg, from about 30 mg to about 100 mg, from about 50 mg to about 80 mg, or about 70 mg of a flavanol, the flavanol being catechin; about 5 mg to about 75 mg, about 10 mg to about 50 mg, about 15 mg to about 30 mg, or about 15.6 mg of caffeine; and about 5 mg to about 80 mg, about 10 mg to about 50 mg, about 15 mg to about 30 mg, or about 13.7 mg of total amino acid;
(iii) from about 3.5 mg to 50 mg of a plant extract, the plant extract composition being a S. officinalis extract composition that comprises rosmarinic acid;
wherein (i) and (ii) comprise a Camellia species and (iii) comprises a Salvia species, wherein the Camellia species are selected from a group consisting of Camellia sinensis, for examples C. sinensis var. sinensis and C. sinensis var. assamica and wherein the Salvia species are selected from a group consisting of S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.

22. The orally consumable composition of any one of claims 9-21, wherein the orally consumable composition comprises about less than 75 mg caffeine but greater than zero mg of caffeine.

23. A method for providing a cognitive benefit to a human subject, comprising orally administering to the subject an orally consumable composition according to any one of claims 1-22.

24. The method of claim 23, wherein the cognitive benefit is selected from a group comprising improving cognitive function; improving mood; improving happiness; improving friendliness; improving sociability; improving relaxation, increasing euphoria; improving conviviality; improving alertness; improving contentedness; improving calmness; improving tranquility; improving mental capability; improving memory accuracy; improving speed of memory; improving relaxation; improving/sustaining attention; improving accuracy of attention; improving stroop effect, improving speed of attention; reducing stress; reducing tension; reducing mental fatigue; reducing anxiety; reducing inertia; reducing headache; promoting maintenance of normal sleep, reducing sleep onset latency, improving sleep quality, alleviating of the subjective feeling of jet leg, or any combinations thereof.

25. The method of claim 24, wherein the method further comprises the step of conducting a cognitive assessment of the subject prior to and/or after the subject has consumed the orally consumable composition of any one of the claims 1-22.
